(19) United States
(12) Patent Application Publication
(10) Pub. No.: US 2026/0034134 A1
(43) Pub. Date: Feb. 5, 2026

(54) TREATMENT OF CANCER WITH METASTASES IN THE CENTRAL NERVOUS SYSTEM

(71) Applicant: Boehringer Ingelheim International GmbH, Ingelheim am Rhein (DE)

(72) Inventors: Mohammad Reza ABBASI, Vienna (AT); Mario AMEND, Albstadt (DE); Behbood SADROLHEFAZI, Oakville (CA); Lukas SCHROETER, Mengen (DE)

(21) Appl. No.: 19/288,168

(22) Filed: Aug. 1, 2025

Related U.S. Application Data
(60) Provisional application No. 63/795,744, filed on Apr. 28, 2025.

(30) Foreign Application Priority Data
Aug. 2, 2024 (EP) 24382872.0
Sep. 6, 2024 (EP) 24382956.1

(51) Int. Cl. A61K 31/519; A61K 9/16; A61K 45/06; A61P 35/04

(52) U.S. Cl. CPC A61K 31/519; A61K 9/16; A61K 45/06; A61P 35/04

(57) ABSTRACT

The present invention relates to the treatment of cancer with metastases in the central nervous system (CNS), in particular in the brain, using the HER2 tyrosine kinase inhibitor zongertinib. Accordingly, compounds for use, methods of prevention and/or treatment and uses related to said treatment are herewith provided.

### RELATED APPLICATION DISCLOSURE

This application claims the benefit pursuant to 35 U.S.C. § 119 of European Patent Application No. 24382872.0, filed Aug. 2, 2024, and European Patent Application No. 24382956.1, filed on Sep. 6, 2024, pending, and this application also claims priority to U.S. Application No. 63/795,744, filed Apr. 28, 2025, each of which is hereby incorporated by reference herein in its entirety.

### FIELD OF THE INVENTION

The present invention relates to the treatment of cancer with metastases in the central nervous system (CNS), in particular in the brain, using the HER2 tyrosine kinase inhibitor zongertinib.

### BACKGROUND OF THE INVENTION

Human epidermal growth factor receptor 2 (HER2; encoded by ERBB2) belongs to the family of ERBB transmembrane receptor tyrosine kinases.

Aberrant activation of HER2 through different mechanisms plays a crucial role in the development and progression of a variety of cancers. HER2 aberrations are associated with a high incidence of CNS metastasis across several cancer types, including breast, gastric, lung and colorectal cancer (Tarantino P. et al., Breast Cancer Research and Treatment (2021) 185:879-881). More than 20% of human breast cancers overexpresses HER2 resulting from ERBB2 amplification and manifests a historically poor prognosis (Swain, S. M., Shastry, M. & Hamilton, E. Nat. Rev. Drug Discov. 22, 101-126; 2023). It is estimated that up to 50% of patients with HER2-positive breast cancer will develop brain metastases when followed longitudinally. Patients who develop brain metastases often have a poor prognosis and a reduced quality of life (Leone, J. P. & Lin, N. U., Current Oncology Reports (2019) 21:49). Also in 12 to 20% of metastatic gastric, gastroesophageal junction or esophageal adenocarcinoma cases, HER2 is overexpressed (Jorgensen J T, Hersom M. J Cancer; 3, p. 137-144; 2012; Wu H, et al. Tumori; 103 (3), p. 249-254; 2017).

In addition to overexpression, HER2 can be aberrantly activated by oncogenic mutations in multiple solid cancers. For instance, HER2 mutations occur in 2-4% of non-small cell lung cancers (NSCLC) and have emerged as important oncogenic drivers (Connell, C. M. & Doherty, G. J. Esmo Open 2, e000279 (2017); Yan, M., et al. Cancer Treat Rev; 40, 770-780 (2014); Stephens, P. et al. Nature 431, 525-526 (2004)). This subset of NSCLC, especially those carrying the most common 12 base pair insertion in ERBB2 exon 20 resulting in the duplication of the amino acids YVMA in HER2 (HER2^{YVMA}), is associated with aggressive disease progression, poor clinical outcomes in response to chemotherapy and immunotherapy and a higher incidence of brain metastases compared with NSCLC without HER2 mutations or with other mutations (Baraibar I, et al. Crit Rev Oncol Hematol 2020; 148:102906 and Li B T, et al. N Engl J Med 2022; 386:241-51). In fact, patients with NSCLC often present brain metastases at diagnosis (˜10-25%) and >25% develop metastases in the central nervous system (CNS) over their disease course (Mantovani C, et al. Front Oncol. 2021; 11:772789). Brain metastases in HER2-mutant NSCLC are associated with a reduced quality of life and poor prognosis (Offin M et al. Cancer 2019; 125:4380-4387).

ERBB signaling can also be aberrantly activated through alterations in the ligands to the receptors of the ERBB family. In this context, fusions of the neuregulin-1 gene (NRG1), although very rare, have been clinically documented (Nagasaka, M. & Ou, S.-H. I. Trends Cancer 8, 242-258 (2022)).

Therefore, there is the need to find safe and efficacious treatment for patients affected by cancer with a metastasis in the CNS, especially where the cancer comprises a HER2 aberration. Zongertinib is a potent and selective covalent tyrosine kinase inhibitor of wild type and mutant HER2 that spares wild type epithelial growth factor receptor (EGFR; also referred to as HER1 and encoded by EGFR), another member of the ERBB family. Zongertinib is described in WO 2021/213800 and in Wilding B., et al.; Cancer Discov; 2024; XX; 1-20. It represents a promising treatment option for HER2 dependent cancers.

### BRIEF DESCRIPTION OF THE FIGURES

FIG. 1. Design of the dose escalation and expansion clinical trial of Example 1, testing zongertinib as monotherapy in patients with HER2 aberration-positive advanced or metastatic solid tumors.

FIG. 2A. Waterfall plot showing best change from baseline in non-CNS target lesions (RECIST v1.1, based on investigator assessment) expressed as a percentage in all patients in Phase Ia (see Examples 1 and 2). Each bar represents a patient with an advanced solid tumor with a HER2 aberration, with confirmed response and ≥1 post-baseline tumor assessment. Lighter bars show patients with lung cancer. Darker bars show patients with a cancer different from lung cancer. The dashed line at 20% delimits progressive disease (above 20%). The dashed line at −30% delimits partial response (PR) (below 30%). The interval between 20% and −30% corresponds to stable disease (SD).

FIG. 2B. Waterfall plot showing best change from baseline in non-CNS target lesions (RECIST v1.1, based on investigator assessment) expressed as a percentage in patients in Phase Ia with brain metastases ((see Examples 1 and 2). Each bar represents a patient with an advanced solid tumor with a HER2 aberration, with confirmed response and ≥1 post-baseline tumor assessment. Lighter bars show patients with lung cancer. Darker bars show patients with a cancer different from lung cancer. The dashed line at 20% delimits progressive disease (above 20%). The dashed line at −30% delimits partial response (PR) (below 30%). The interval between 20% and −30% corresponds to stable disease (SD).

FIG. 3A. CT scans from a patient with baseline brain metastases from cohort 1 of Phase Ib (see Examples 1 and 3). FIG. 3A shows the CT scan at baseline. Left panels: lung; middle panels: adrenal; right panels: brain.

FIG. 3B. CT scans from a patient with baseline brain metastases from cohort 1 of Phase Ib (see Examples 1 and 3). FIG. 3B shows the CT scan of the patient shown in FIG. 3A after 7 months of treatment with zongertinib (240 mg QD). Left panels: lung; middle panels: adrenal; right panels: brain.

FIG. 4A. CT scans from a patient with baseline brain metastases from cohort 1 of Phase Ib (see Examples 1 and 3). FIG. 4A shows CT scan at baseline.

FIG. 4B. CT scans from a patient with baseline brain metastases from cohort 1 of Phase Ib (see Examples 1 and 3). FIG. 4B shows the CT scan of the patient shown in FIG. 4A after 6 weeks of treatment with zongertinib (240 mg QD).

FIG. 5A. Scans from a patient with baseline brain metastases from cohort 1 of Phase Ib (see Examples 1 and 4). FIG. 5A shows the CT scan at baseline.

FIG. 5B. Scans from a patient with baseline brain metastases from cohort 1 of Phase Ib (see Examples 1 and 4). FIG. 5B shows the CT of the patient shown in FIG. 5A scan after 12 weeks of treatment with zongertinib (120 mg QD).

FIG. 6. Spider plot showing tumor shrinkage in the CNS, defined as the percentage change from baseline in the sum of longest diameter of CNS target lesions according to RANO-BM per the investigator's assessment, over time for all patients with target lesions in the brain per RANO-BM in Phase Ia (see Examples 1 and 6) treated with different doses of zongertinib. Patients with target lesions in the brain per RANO-BM are patients for whom baseline and post-baseline brain lesion measurements were performed by the investigator. Negative values indicate a decrease in CNS tumor lesion size from baseline. Each line represents a patient. Intervals defined by the dashed lines have the meanings defined in FIGS. 2A and 2B.

FIG. 7A. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohort 1 of Phase Ib (see Examples 1 and 7) treated with 120 mg QD zongertinib. Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 7B. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohort 1 of Phase Ib (see Examples 1 and 7) treated with 240 mg QD zongertinib. Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 8A. Swimmer plot showing overall response by RANO-BM intracranial efficacy assessments (central review) over time for patients with brain lesions at baseline in cohort 1 of Phase Ib (see Examples 1 and 7) treated with 120 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 7.

FIG. 8B. Swimmer plot showing overall response by RANO-BM intracranial efficacy assessments (central review) over time for patients with brain lesions at baseline in cohort 1 of Phase Ib (see Examples 1 and 7) treated with 240 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 7.

FIG. 9A. Swimmer plot showing overall response by CNS RECIST 1.1 intracranial efficacy assessments (central review) over time for patients with RECIST 1.1 measurable brain lesions at baseline in cohort 1 of Phase Ib (see Examples 1 and 7) treated with 120 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 7.

FIG. 9B. Swimmer plot showing overall response by CNS RECIST 1.1 intracranial efficacy assessments (central review) over time for patients with RECIST 1.1 measurable brain lesions at baseline in cohort 1 of Phase Ib (see Examples 1 and 7) treated with 240 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 7.

FIG. 10. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohort 4 of Phase Ib (see Examples 1 and 8) treated with 120 mg QD zongertinib. Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 11A. Swimmer plot showing overall response by RANO-BM intracranial efficacy assessments (central review) over time in patients with brain lesions at baseline in cohort 4 of Phase Ib (see Examples 1 and 8) treated with 120 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 8.

FIG. 11B. Swimmer plot showing overall response by RANO-BM intracranial efficacy assessments (central review) over time in patients with brain lesions at baseline in cohort 4 of Phase Ib (see Examples 1 and 8) treated with 240 mg QD zongertinib. Each bar represents a patient. The length of the bars represents progression-free survival times and not necessarily treatment durations, i.e., the bars end at progression or censoring events even in cases where the patient remained on treatment after this event. Arrows at the end of the bars indicate the patient still has ongoing progression-free survival at the data-cut of Example 8.

FIG. 12A. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohorts 1 or 4 of Phase Ib (see Examples 1 and 11) treated with 120 mg QD zongertinib. Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 12B. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohorts 1 or 4 of Phase Ib (see Examples 1 and 11) treated with 240 mg QD zongertinib. Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 13A. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohorts 1 or 4 of Phase Ib, who had not received prior radiotherapy for CNS lesions, treated with 120 mg QD zongertinib (see Examples 1 and 11). Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

FIG. 13B. Waterfall plot showing best tumor shrinkage in the CNS, defined as the best percentage change from baseline in the sum of longest diameter of CNS target lesions according to RANO-BM per the investigator's assessment, for patients with target lesions in the brain per RANO-BM in cohorts 1 or 4 of Phase Ib, who had not received prior radiotherapy for CNS lesions, treated with 240 mg QD zongertinib (see Examples 1 and 11). Patients with target lesions in the brain per RANO-BM are patients for whom baseline and post-baseline brain lesion measurements were performed by the investigator. Negative values indicate a decrease in CNS tumor lesion size from baseline. Each bar represents a patient. Intervals defined by the dashed lines have the meanings defined in FIGS. 2A and 2B.

### SUMMARY OF THE INVENTION

A first aspect relates to zongertinib or a pharmaceutically acceptable salt thereof for use in the prevention and/or treatment of cancer, wherein the cancer comprises at least one metastasis in the central nervous system. Preferably, zongertinib or the pharmaceutically acceptable salt thereof is effective on said at least one metastasis. Preferably, zongertinib or the pharmaceutically acceptable salt thereof approximately maintains or decreases the volume of said metastasis over a period of time between a first assessment and a later assessment.

Another aspect relates to a method of treating and/or preventing cancer in a patient in need thereof, wherein the method comprises administering to the patient zongertinib or a pharmaceutically acceptable salt thereof, wherein the cancer comprises at least one metastasis in the central nervous system.

Another aspect relates to a use of zongertinib or a pharmaceutically acceptable salt thereof for the manufacture of a medicament for the treatment and/or prevention of cancer, wherein the cancer comprises at least one metastasis in the central nervous system.

A further aspect relates to zongertinib or a pharmaceutically acceptable salt thereof for use in the prevention and/or treatment of a metastasis in the central nervous system.

Another aspect relates to a method of treating and/or preventing a metastasis in the central nervous system in a patient in need thereof, wherein the method comprises administering to the patient zongertinib or a pharmaceutically acceptable salt thereof.

Another aspect relates to a use of zongertinib or a pharmaceutically acceptable salt thereof for the manufacture of a medicament for the treatment and/or prevention of a metastasis in the central nervous system.

In embodiments of zongertinib for use, of the method or of the use, the metastasis in the central nervous system is a brain metastasis. In other words, it is preferred that the cancer comprises at least one brain metastasis.

In embodiments of zongertinib for use, of the method or of the use, the cancer is selected from the group consisting of: brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In embodiments of zongertinib for use, of the method or of the use, the cancer is lung cancer or breast cancer.

In embodiments of zongertinib for use, of the method or of the use, the cancer is non-small cell lung cancer (NSCLC).

In embodiments of zongertinib for use, of the method or of the use, the cancer comprises a HER2 aberration.

In embodiments of zongertinib for use, of the method or of the use, the cancer is HER2 mutant.

In embodiments of zongertinib for use, of the method or of the use, the cancer comprises a mutation in the tyrosine kinase domain of HER2.

In embodiments of zongertinib for use, of the method or of the use, the metastasis in the central nervous system is asymptomatic, symptomatic, stable or active.

Another aspect relates to zongertinib or a pharmaceutically acceptable salt thereof for use in the prevention and/or treatment of metastatic cancer, wherein the metastatic cancer comprises at least one lesion in the central nervous system.

Another aspect relates to a method of treating and/or preventing metastatic cancer in a patient in need thereof, wherein the method comprises administering to the patient zongertinib or a pharmaceutically acceptable salt thereof, wherein the metastatic cancer comprises at least one lesion in the central nervous system.

Another aspect relates to a use of zongertinib or a pharmaceutically acceptable salt thereof for the manufacture of a medicament for the treatment and/or prevention of metastatic cancer, wherein the metastatic cancer comprises at least one lesion in the central nervous system.

In embodiments of zongertinib for use, of the method or of the use, the lesion in the central nervous system is a brain lesion. In other words, it is preferred that the metastatic cancer comprises at least one brain lesion.

Another aspect relates to zongertinib or a pharmaceutically acceptable salt thereof for use in the prevention and/or treatment of metastatic cancer, wherein the metastatic cancer comprises or is metastatic cancer to the central nervous system.

Another aspect relates to a method of treating and/or preventing metastatic cancer in a patient in need thereof, wherein the method comprises administering to the patient zongertinib or a pharmaceutically acceptable salt thereof, wherein the metastatic cancer comprises or is metastatic cancer to the central nervous system.

Another aspect relates to a use of zongertinib or a pharmaceutically acceptable salt thereof for the manufacture of a medicament for the treatment and/or prevention of metastatic cancer, wherein the metastatic cancer comprises or is metastatic cancer to the central nervous system.

In embodiments of zongertinib for use, of the method or of the use, the metastatic cancer comprises or is metastatic cancer to the brain.

In embodiments of zongertinib for use, of the method or of the use, the metastatic cancer is selected from the group consisting of: metastatic brain cancer, metastatic breast cancer, metastatic biliary tract cancer, metastatic bladder cancer, metastatic cervical cancer, metastatic uterine cancer, metastatic colorectal cancer, metastatic endometrial cancer, metastatic ovarian cancer, metastatic skin cancer, metastatic gastric cancer, esophagus tumor, metastatic head and neck tumor, metastatic salivary gland cancer, metastatic gastrointestinal cancer, metastatic small bowel cancer, metastatic gallbladder tumor, metastatic kidney cancer, metastatic liver cancer, metastatic lung cancer and metastatic prostate cancer.

In embodiments of zongertinib for use, of the method or of the use, the metastatic cancer is metastatic lung cancer or metastatic breast cancer.

In embodiments of zongertinib for use, of the method or of the use, the metastatic cancer is metastatic non-small cell lung cancer (NSCLC).

In embodiments of zongertinib for use, of the method or of the use, the metastatic cancer comprises a HER2 aberration.

In embodiments of zongertinib for use, of the method or of the use, the metastatic cancer is HER2 mutant.

In embodiments of zongertinib for use, of the method or of the use, the metastatic cancer comprises a mutation in the tyrosine kinase domain of HER2.

In embodiments of zongertinib for use, of the method or of the use, the metastatic cancer and/or the lesion is asymptomatic, symptomatic, stable or active.

In embodiments of zongertinib for use, of the method or of the use, zongertinib or the pharmaceutically acceptable salt thereof is administered as second or further line therapy.

In embodiments of zongertinib for use, of the method or of the use, zongertinib or the pharmaceutically acceptable salt thereof is administered following a systemic anti-cancer therapy.

In embodiments of zongertinib for use, of the method or of the use, zongertinib or the pharmaceutically acceptable salt thereof is administered in a daily dose of 120 mg, 180 mg, 200 mg, 240 mg, 300 mg, 360 mg, 400 mg, 420 mg, 480 mg, 500 mg, 540 mg or 600 mg.

In embodiments of zongertinib for use, of the method or of the use, zongertinib or the pharmaceutically acceptable salt thereof is administered in a daily dose of 120 mg or 240 mg. In embodiments of zongertinib for use, of the method or of the use, zongertinib or the pharmaceutically acceptable salt thereof is administered as monotherapy.

In embodiments of zongertinib for use, of the method or of the use, zongertinib or the pharmaceutically acceptable salt thereof is administered in combination with a combination partner.

In embodiments of zongertinib for use, of the method or of the use, zongertinib or the pharmaceutically acceptable salt thereof is administered as a spray-dried dispersion.

It is to be understood that any of the embodiments or aspects disclosed herein referring to compatible features (e.g. definition of the cancer by location and/or HER2 aberration, daily dose of zongertinib, once/twice daily administration, administration further to systemic anti-cancer therapy agent, second/further line administration, combination with additional intervention etc.) may be combined with each other to provide further embodiments of the invention.

### DETAILED DESCRIPTION OF THE INVENTION

It is a purpose of the present invention to treat cancer with metastases in the central nervous system (CNS), in particular in the brain.

It has been found that zongertinib can penetrate the blood-brain barrier in patients with CNS metastases. Zongertinib has shown clinically meaningful activity in patients with brain metastases, also in terms of controlling/reducing the size of brain metastases, demonstrating promising efficacy in patients with HER2 aberration-positive solid tumors, including in those with brain metastases at baseline, while maintaining a manageable safety profile.

Zongertinib was and is being administered to patients having HER2 aberrant cancers and CNS metastases in different clinical trials, three of which are described in the Examples below. Example 1 describes the protocol of a Phase I clinical trial with a dose escalation part (Phase Ia) and a dose expansion part (Phase Ib), which in turn comprises different cohorts defining different patient populations. The Phase Ia enrolls patients with HER2 aberrant solid tumors, including different types of aberrations, cancers and doses of zongertinib. The Phase Ib enrolls patients with HER2 mutant NSCLC, with cohort 4 focusing on patients with active/symptomatic brain metastases. The other cohorts allowed for the inclusion of patients with asymptomatic brain lesions. In Phase Ib, most patients received zongertinib at 120 mg once daily. Results of Example 1 are presented in Examples 2 to 11. In Phase Ia, the confirmed intracranial overall response rate (ORR) and disease control rate (DCR) were 16.7% and 76.7% (RANO-BM), respectively, including 2 complete responses (CR) in brain lesions (Example 6). In the study, efficacy was measured systemically (by RECIST 1.1) and locally in the CNS (by RANO-BM and CNS RECIST 1.1). The confirmed systemic ORR in cohort 1 patients with brain metastases was 64%, while the intracranial confirmed ORR was 39% by RANO-BM and 50% by RECIST 1.1. Remarkably, the DCR was 79% by RANO-BM and 100% by RECIST 1.1. Four CRs were observed by RANO-BM (Example 7). Specifically, in patients with symptomatic CNS metastases (cohort 4), the confirmed intracranial ORR and DCR were 43.3% and 86.6%, respectively, by RANO-BM. The observed efficacy was also durable, as demonstrated by median duration of response (DoR) and progression-free survival (PFS) of 6.9 and 8.2 months, respectively, by RANO-BM (Example 8). The efficacy was observed regardless of whether the patient received prior radiotherapy (Example 9). Zongertinib showed comparably meaningful intracranial activity across different dose groups, including 120 mg and 240 mg, as indicated by similar percentages of progressions involving CNS lesions across doses (Example 10). Further, pooled analyses of patients from cohorts 1 and 4 indicate that zongertinib is comparably active in patients with symptomatic and asymptomatic brain metastases and patients with and without prior brain radiotherapy treatment (Example 11). Across phases and cohorts, the safety profile remained manageable.

Example 12 describes a Phase II clinical trial to evaluate zongertinib monotherapy in several HER2 aberrant cancers. Example 13 describes a Phase Ib dose escalation and Phase II dose optimisation clinical trial of zongertinib monotherapy or in combination with other agents in patients with advanced HER2+ metastatic breast cancer (mBC) or metastatic gastric, gastroesophageal junction, or esophageal adenocarcinoma (mGEAC). Patients with brain metastases are eligible for the trials of Examples 12 and 13.

### Zongertinib

As used herein, “zongertinib” refers to the compound as defined below or to a pharmaceutically acceptable salt thereof:

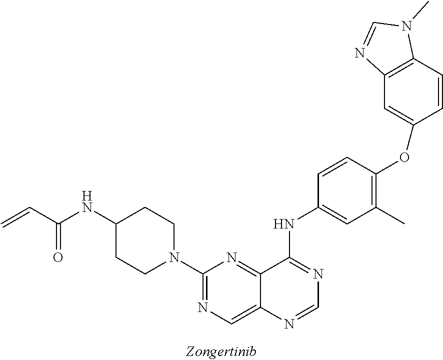 [SMILES: C=CC(=O)NC1CCN(c2ncc3ncnc(Nc4ccc(Oc5ccc6c(c5)ncn6C)c(C)c4)c3n2)CC1]

The IUPAC name of zongertinib is N-{1-[8-({3-methyl-4-[(1-methyl-1H-1,3-benzodiazol-5-yl)oxy]phenyl}amino)-[1,3]diazino[5,4-d]pyrimidin-2-yl]piperidin-4-yl}prop-2-enamide. In case of discrepancy between IUPAC name and depicted formula, the formula shall prevail. WO 2021/213800 discloses zongertinib as example compound I-01 and provides a procedure for its synthesis. Properties of zongertinib and evidence for inhibitory effect on HER2 wild-type and YVMA kinase activity, while sparing EGFR, are also disclosed in WO 2021/213800, which is herein incorporated by reference in its entirety.

Zongertinib as used herein also encompasses any tautomers, pharmaceutically acceptable salts, solid state forms of the compound, as well as solvates, including hydrates and solvates of pharmaceutically acceptable salts thereof.

In embodiments, zongertinib is a free base. Therefore, in any aspect or embodiment, the expression “zongertinib or a pharmaceutically acceptable salt thereof” can be replaced by “zongertinib”, without a reference to the pharmaceutically acceptable salt thereof. In embodiments, pharmaceutically acceptable salts of zongertinib are used. The term “pharmaceutically acceptable” used herein refers to compounds, materials, compositions and/or dosage forms which are, within the scope of sound medical judgement, suitable for use in contact with the tissues of human beings without excessive toxicity, irritation, allergic response, or other problem or complication, and commensurate with a reasonable benefit/risk ratio.

As used herein, “pharmaceutically acceptable salts” of zongertinib refers to zongertinib wherein the compound is modified by making acid or base salts thereof. The term pharmaceutically acceptable salts as used herein generally includes both acid and base addition salts. Pharmaceutically acceptable acid addition salts refer to those salts which retain the biological effectiveness and properties of the free base and which are not biologically or otherwise undesirable, formed with inorganic acids or organic acids. Pharmaceutically acceptable base addition salts include salts derived from inorganic bases or organic nontoxic bases. Examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethane sulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. In embodiments, pharmaceutically acceptable salts are selected from chloride and fumarate salts.

Pharmaceutically acceptable salts can be synthesized from zongertinib by conventional chemical methods. Generally, such salts can be prepared by reacting the free base form of zongertinib with a sufficient amount of the appropriate acid or base in water or in an organic diluent or solvent like ether, ethyl acetate, ethanol, isopropanol, or acetonitrile, or a mixture thereof.

The term “solvate” as used herein refers to an association or complex of one or more solvent molecules and zongertinib. Examples of solvents include water, isopropanol, ethanol, methanol, dimethyl sulfoxide (DMSO), ethyl acetate, acetic acid, tert-butyl methyl ether, tetrahydrofuran, methylethyl ketone, N-methylpyrrolidone and ethanolamine. The term “hydrate” refers to a complex where the solvent molecule is water.

In embodiments, zongertinib is in an amorphous form. In embodiments, zongertinib is in a crystalline form, for example in one of the crystalline forms described in WO 2024/133302, which is herein incorporated by reference in its entirety.

### Cancer

In embodiments, the cancer is one of the following, without being restricted thereto (in other words, the primary cancer of the metastatic cancer is one of the following, without being restricted thereto):

Cancers/tumors/carcinomas of the head and neck: e.g. tumors/carcinomas/cancers of the nasal cavity, paranasal sinuses, nasopharynx, oral cavity (including lip, gum, alveolar ridge, retromolar trigone, floor of mouth, tongue, hard palate, buccal mucosa), oropharynx (including base of tongue, tonsil, tonsillar pilar, soft palate, tonsillar fossa, pharyngeal wall), middle ear, larynx (including supraglottis, glottis, subglottis, vocal cords), hypopharynx, salivary glands (including minor salivary glands);
cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer);
neoplasms of the mediastinum: e.g. neurogenic tumors (including neurofibroma, neurilemoma, malignant schwannoma, neurosarcoma, ganglioneuroblastoma, ganglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma);
cancers/tumors/carcinomas of the gastrointestinal (GI) tract: e.g. tumors/carcinomas/cancers of the esophagus, stomach (gastric cancer), pancreas, liver and biliary tree (including hepatocellular carcinoma (HCC), e.g. childhood HCC, fibrolamellar HCC, combined HCC, spindle cell HCC, clear cell HCC, giant cell HCC, carcinosarcoma HCC, sclerosing HCC; hepatoblastoma; cholangiocarcinoma; cholangiocellular carcinoma; hepatic cystadenocarcinoma; angiosarcoma, hemangioendothelioma, leiomyosarcoma, malignant schwannoma, fibrosarcoma, Klatskin tumor), gall bladder, extrahepatic bile ducts, small intestine (including duodenum, jejunum, ileum), large intestine (including cecum, colon, rectum, anus; colorectal cancer, gastrointestinal stroma tumor (GIST)), appendix, genitourinary system (including kidney, e.g. renal pelvis, renal cell carcinoma (RCC), nephroblastoma (Wilms' tumor), hypernephroma, Grawitz tumor; ureter; urinary bladder, e.g. urachal cancer, urothelial cancer; urethra, e.g. distal, bulbomembranous, prostatic; prostate (androgen dependent, androgen independent, castration resistant, hormone independent, hormone refractory), penis);
cancers/tumors/carcinomas of the testis: e.g. seminomas, non-seminomas;
Gynecologic cancers/tumors/carcinomas: e.g. tumors/carcinomas/cancers of the ovary, fallopian tube, peritoneum, cervix, vulva, vagina, uterine body (including endometrium, fundus);
cancers/tumors/carcinomas of the breast: e.g. mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), HER2 positive breast cancer, triple negative breast cancer, Paget's disease of the breast;
cancers/tumors/carcinomas of the endocrine system: e.g. tumors/carcinomas/cancers of the endocrine glands, thyroid gland (thyroid carcinomas/tumors; papillary, follicular, anaplastic, medullary), parathyroid gland (parathyroid carcinoma/tumor), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; nonfluorineunctional PET, PPoma, gastrinoma, insulinoma, VIPoma, glucagonoma, somatostatinoma, GRFoma, ACTHoma), carcinoid tumors;
sarcomas of the soft tissues: e.g. fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma, epithelioid sarcoma, extrarenal rhabdoid tumor, desmoplastic small cell tumor;
sarcomas of the bone: e.g. myeloma, reticulum cell sarcoma, chondrosarcoma (including central, peripheral, clear cell, mesenchymal chondrosarcoma), osteosarcoma (including parosteal, periosteal, high-grade surface, small cell, radiation-induced osteosarcoma, Paget's sarcoma), Ewing's tumor, malignant giant cell tumor, adamantinoma, (fibrous) histiocytoma, fibrosarcoma, chordoma, small round cell sarcoma, hemangioendothelioma, hemangiopericytoma, osteochondroma, osteoid osteoma, osteoblastoma, eosinophilic granuloma, chondroblastoma;
mesothelioma: e.g. pleural mesothelioma, peritoneal mesothelioma;
cancers of the skin: e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer;
neoplasms of the central nervous system and brain: e.g. astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors;
peripheral nervous system cancer;
lymphomas and leukemias: e.g. B-cell non-Hodgkin lymphomas (NHL) (including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T-cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (BchlorineL), chronic T-cell lymphocytic leukemia (TchlorineL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML);
cancers of unknown primary site (CUP).

All cancers/tumors/carcinomas mentioned above which are characterized by their specific location/origin in the body are meant to include both the primary tumors and the metastatic tumors derived therefrom. Preferably, the cancer as defined herein (including in any embodiment referring to e.g. cancer types) is metastatic, advanced, and/or unresectable, in particular, metastatic.

All cancers/tumors/carcinomas mentioned above may be further differentiated by their histopathological classification:
Epithelial cancers, e.g. squamous cell carcinoma (SCC) (carcinoma in situ, superficially invasive, verrucous carcinoma, pseudosarcoma, anaplastic, transitional cell, lymphoepithelial), adenocarcinoma (AC) (well-differentiated, mucinous, papillary, pleomorphic giant cell, ductal, small cell, signet-ring cell, spindle cell, clear cell, oat cell, colloid, adenosquamous, mucoepidermoid, adenoid cystic), mucinous cystadenocarcinoma, acinar cell carcinoma, large cell carcinoma, small cell carcinoma, neuroendocrine tumors (small cell carcinoma, paraganglioma, carcinoid); oncocytic carcinoma;
Nonepithilial cancers, e.g. sarcomas (fibrosarcoma, chondrosarcoma, rhabdomyosarcoma, leiomyosarcoma, hemangiosarcoma, giant cell sarcoma, lymphosarcoma, fibrous histiocytoma, liposarcoma, angiosarcoma, lymphangiosarcoma, neurofibrosarcoma), lymphoma, melanoma, germ cell tumors, hematological neoplasms, mixed and undifferentiated carcinomas. In general and unless stated otherwise, a reference to a cancer by its site (e.g. breast cancer) is intended to refer in particular to the site of the primary cancer of the metastatic cancer.

In some embodiments, the cancer is a solid tumor. In some embodiments, the cancer is manifested by at least one solid tumor.

In some embodiments, the cancer is selected from the group consisting of brain cancer, breast cancer, endocrine cancer, gastrointestinal cancer, gynecologic cancer, head and neck tumor, lung cancer, nervous system cancer, and skin cancer.

Preferably, said brain cancer is a glioblastoma or a glioma.

Preferably, said breast cancer is lobular breast cancer. In addition or in alternative, said breast cancer is preferably metastatic.

Preferably, said endocrine cancer is nerve sheath tumor, more preferably HER2 mutant nerve sheath tumor.

Preferably, said gastrointestinal cancer is selected from the group consisting of anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer and small bowel cancer.

In addition or in alternative, said gastrointestinal cancer may be a gastrointestinal neuroendocrine tumor, preferably HER2 mutant. Still preferably, said gastrointestinal cancer is selected from the group consisting of gastric adenocarcinoma, gastroesophageal junction adenocarcinoma and esophageal adenocarcinoma, in particular metastatic gastric adenocarcinoma, metastatic gastroesophageal junction adenocarcinoma and metastatic esophageal adenocarcinoma.

Preferably, said gynecologic cancer is selected from the group consisting of cervical cancer, uterine cancer, endometrial cancer and ovarian cancer.

As used herein, “head and neck tumor” preferably refers to a head and neck cancer.

Preferably, said head and neck tumor is a salivary gland cancer or tumor.

Preferably, said lung cancer is non-small cell lung cancer (NSCLC).

Preferably, said nervous system cancer is peripheral nervous system cancer, more preferably HER2 amplified peripheral nervous system cancer.

Preferably, said skin cancer is not a melanoma, i.e. non-melanoma skin cancer.

In some embodiments, the cancer is selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer. In some embodiments, the cancer is HER2 overexpressed, HER2 amplified and/or HER2 mutant (in particular HER2 exon 20 mutant) cancer selected from the group consisting of glioblastoma, glioma, lobular breast cancer, metastatic breast cancer, nerve sheath tumor, anal cancer, appendix cancer, biliary tract cancer, bladder cancer, colorectal cancer, esophagogastric cancer, gastric cancer, esophagus tumor, gastroesophageal cancer, gallbladder tumor, hepatobiliary cancer, kidney cancer, liver cancer, pancreatic cancer, prostate cancer, small bowel cancer, neuroendocrine gastrointestinal cancer, metastatic gastric adenocarcinoma, metastatic gastroesophageal junction adenocarcinoma, metastatic esophageal adenocarcinoma, cervical cancer, uterine cancer, endometrial cancer, ovarian cancer, salivary gland cancer, non-small cell lung cancer (NSCLC), peripheral nervous system cancer and non-melanoma skin cancer.

In some embodiments, the cancer is selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In some embodiments, the cancer is HER2 overexpressed, HER2 amplified and/or HER2 mutant (in particular HER2 TKD mutant, more in particular exon 20 mutant) cancer selected from the group consisting of brain cancer, breast cancer, biliary tract cancer, bladder cancer, cervical cancer, uterine cancer, colorectal cancer, endometrial cancer, ovarian cancer, skin cancer, gastric cancer, esophagus tumor, head and neck tumor, salivary gland cancer, gastrointestinal cancer, small bowel cancer, gallbladder tumor, kidney cancer, liver cancer, lung cancer and prostate cancer.

In further embodiments, the cancer is selected from cancers/tumors/carcinomas of the lung: e.g. non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer). In still further embodiments, the cancer is NSCLC. In still further embodiments, the cancer is HER2 TKD mutant NSCLC. In still further embodiments, the cancer is HER2 exon 20 mutant NSCLC.

In another aspect, the cancer is selected from the group consisting of breast cancer, esophageal cancer, gastric cancer, gastroesophageal junction cancer, lung cancer and ovarian cancer. In another aspect, the cancer is selected from the group consisting of breast cancer, esophageal cancer, gastric cancer, gastroesophageal junction cancer, and lung cancer. Preferably, said lung cancer is non-small cell lung cancer (NSCLC).

In further embodiments, the cancer is selected from the group consisting of lung cancer and breast cancer. Preferably, said lung cancer is NSCLC.

In another aspect, said cancer is advanced, unresectable and/or metastatic.

In a further preferred embodiment, the cancer comprises at least one metastasis in the CNS and at least one metastasis outside the CNS. Metastases outside the CNS could be located for example in the lung, lymph node, brain, bone or liver.

In another aspect, said cancer is an adenocarcinoma.

In embodiments, the cancer or tumor comprises a HER2 aberration. This means that the cells of the cancer or tumor harbor an aberration of HER2. As used herein, the expressions “HER2 aberration”, “aberration of HER2” and grammatical variants thereof have the meaning commonly attributed to them in the art and include any variation or alteration in the HER2 protein or its encoding gene, such as: overexpression of the HER2 protein, amplification of the HER2-encoding gene, mutations in the HER2-encoding gene and/or in the HER2 protein (in particular non-synonymous mutations, somatic mutations, mutations in specific regions, e.g. in the tyrosine kinase domain, in exon 20, etc.) as well as gene rearrangements of HER2 and/or NRG1. When the cancer comprises a HER2 aberration, it can be referred to as HER2 aberrant. When the cancer comprises an overexpression of the HER2 protein, it can be referred to as HER2 overexpressed. When the cancer comprises an amplification of the HER2-encoding gene, it can be referred to as HER2 amplified. When the cancer comprises a mutation in the HER2-encoding gene and/or in the HER2 protein, it can be referred to as HER2 mutant.

In embodiments, the cancer is HER2 overexpressed, HER2 amplified and/or HER2 mutant.

In embodiments, the cancer comprises a mutation in the tyrosine kinase domain of HER2, in particular an activating mutation in the tyrosine kinase domain of HER2. In embodiments, the cancer is HER2 exon 20 mutant cancer.

In embodiments, the cancer comprises a gene rearrangement of HER2 and/or NRG1.

The presence or absence of HER2 alterations, including overexpression, amplification or mutations, can be determined using methods known in the art.

“HER2 overexpressed” as used herein refers to a cancer, comprising cells that express HER2 at levels detectable by immunohistochemistry (e.g. IHC 2+ and IHC 3+) and/or methods assaying ERBB2 messenger RNA.

“HER2 amplified” as used herein refers to a cancer, comprising cells exhibiting more than 2, in particular more than 3, 4, 5, 6, 7, 8, 9 or 10, preferably more than 6, copies of the HER2 gene ERBB2.

HER2 expression, gene copy number and amplification can be measured, for example, by determining nucleic acid sequencing (e.g., sequencing of genomic DNA or cDNA), measuring mRNA expression, measuring protein abundance, or a combination thereof. HER2 testing methods include immunohistochemistry (IHC), in situ hybridization—including fluorescence in situ hybridization (FISH), chromogenic in situ hybridization (CISH)—ELISAs, and RNA quantification using techniques such as Reverse Transcription-Polymerase Chain Reaction (RT-PCR), microarray analysis and Next Generation Sequencing (NGS). HER2 expression in or on the cancer sample cells can be compared to a reference cell. The reference cell can be a non-cancer cell obtained from the same subject as the sample cell. The reference cell can be a non-cancer cell obtained from a different subject or a population of subjects.

When the cancer is HER2 overexpressed and/or HER2 amplified in or on a cell, the cancer can be referred to as being “HER2 positive”. The level of HER2 amplification or overexpression in HER2 positive cancers is commonly expressed as a score ranging from 0 to 3 (i.e., HER2 0, HER2 1+, HER2 2+, or HER2 3+), with higher scores corresponding to greater degrees of expression.

In an aspect, the cancer is HER2 positive.

Preferably, “HER2 overexpressed”, “HER2 amplified” and “HER2 positive” mean that the cancer comprises cells having an immunohistochemistry score of 2+ or 3+.

Preferably, “HER2 overexpressed”, “HER2 amplified” and “HER2 positive” mean that the cancer comprises cells having HER2 amplification defined by in situ hybridization.

In some embodiments, the HER2 status of the cancer, specifically of a sample cell within the cancer, is determined. The determination can be made before the combination treatment begins, during treatment, or after treatment has been completed. In some instances, determination of the HER2 status results in a decision to change therapy (e.g., switching to a different anti-HER2 antibody or anti-HER2 ADC or switching from another treatment method to a method of the present invention).

The sample cell can be obtained as a biopsy specimen, by surgical resection, or as a fine needle aspirate (FNA).

In some embodiments, the sample cell is determined to be HER2 positive when HER2 is expressed at a higher level in the sample cell compared to a reference cell. In some embodiments, the cell is determined to be HER2 positive when HER2 is overexpressed at least about 1.5-fold (e.g., about 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 1 1-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or more) compared to a reference cell. In particular embodiments, the cell is determined to be HER2 positive when HER2 is overexpressed at least about 1.5-fold compared to the reference cell.

In some embodiments, the sample cell is determined to be HER2 positive when the FISH or CISH signal ratio is greater than 2.

“HER2 mutant” as used herein refers to a cancer harboring at least one mutation, i.e. an alteration in the nucleic acid sequence of the HER2-encoding gene and/or an alteration in the amino acid sequence of the HER2 protein, including but not limited to those listed below. Mutations can be found with any method known to the skilled person, such as molecular diagnostic methods including but not limited to Polymerase Chain Reaction (PCR), Single Strand Conformational Polymorphism (SSCP), Denaturing Gradient Gel Electrophoresis (DGGE), Heteroduplex analysis, Restriction fragment length polymorphism (RFLP), Next Generation Sequencing (NGS) and Whole Exome Sequencing. Preferably, the mutation in the HER2 mutant cancer is an activating mutation. In other words, preferably, the HER2 mutant cancer comprises a HER2 activating mutation.

In embodiments of said HER2 mutant cancer, the mutation is a non-synonymous mutation. As used herein, the term “non-synonymous” has the meaning commonly attributed to it in the art and in particular refers to a mutation in the nucleic acid sequence of the HER2-encoding gene that alters the amino acid sequence of the HER2 protein.

In embodiments of said HER2 mutant cancer, the mutation is a somatic mutation. As used herein, the term “somatic” has the meaning commonly attributed to it in the art and in particular refers to a mutation in the nucleic acid sequence of the HER2-encoding gene occurring in a cell other than a gamete, a germ cell or a gametocyte.

In embodiments of said HER2 mutant cancer, the mutation is a non-synonymous somatic mutation.

In embodiments of said HER2 mutant cancer, the mutation is a non-synonymous somatic mutation in the tyrosine kinase domain of HER2.

In embodiments of said HER2 mutant cancer, the mutation is in the tyrosine kinase domain of HER2, in particular in the exon 20 of HER2. In the latter case, the cancer can be referred to as HER2 exon 20 mutant. In these embodiments, the mutation is preferably a HER2 activating mutation in the tyrosine kinase domain of HER2, in particular in the exon 20 of HER2. As used herein, “HER2 mutant” may also refer to a rearrangement involving the HER2 gene ERBB2 and/or the NRG1 gene.

As used herein, a cancer comprising a mutation in the tyrosine kinase domain of HER2 is a cancer where the cancer or tumor cells harbour at least one mutation in the tyrosine kinase domain of HER2, which ranges from amino acids 694 to 883 and/or exons 18 to 21.

“Cancer with HER2 exon 20 mutation” or “HER2 exon 20 mutant cancer” as used herein refers to a cancer where the cancer or tumor cells harbour at least one HER2 exon 20 mutation including but not limited to the mutations listed below.

ERBB2 (HER2) exon 20 encodes for a part of the kinase domain and ranges from amino acids 769 to 835. Every mutation, insertion, duplication or deletion within this region is defined as an exon 20 mutation including the following mutations: p.A772_G773insMMAY; p.Y772_A775_dup (YVMA); p.A775_G776ins YVMA; p.Y772ins YVMA; p.M774delinsWLV; p.A775_G776insSVMA; p.A775_G776ins VVMA; p.A775_G776insYVMS; p.A775_G776insC; p.A776_delinsVC; p.A776_delinsLC; p.A776_delinsVV; p.A776_delinsAVGC; p.A776_delinsIC; p.A776_V777delinsCVC; p.V777_insE; p.V777_G778insV; p.V777_G778insC; p.V777_G778insCG; p.V777_S779dup; p.V777L; p.V777M; p.G778_P780dup (GSP); p.G778_S779insCPG; p.G778_S779insG; p.G776_delinsVC; p.G776_V777delinsAVGCV; p.G776delinsLC; p.G776_V777delinsAVCV; p.G776delinsVV; p.G776_V777insL; p.G776_V777insVGC; p.G776C; p.G776A; p.G776L; p.G776V; p.P780_Y78linsGSP (“p.” is referring to the HER2 protein).

In addition HER2 mutations exist outside of exon 20 including the following mutations: p.S310A; p.S310F; p.S310Y; p.R678Q; p.G727A; p.T733I; p.L755S; p.L755A; p.L755F; p.L755P; p.V842I; p.D769Y; p.D769H; p.R103Q; p.G1056S; p.I767M; p.L869R; p.L869R; p.T733I; p.T862A; p.V697L; p.R929W; p.D277H; p.D277Y; p.G660D (“p.” is referring to the HER2 protein).

Of these, examples of tyrosine kinase mutations include: p.G727A; p.T733I; p.L755S; p.L755A; p.L755F; p.L755P; p.L755S; p.V842I; p.D769Y; p.D769H; p.I767M; p.L869R; p.L869R; p.T733I; p.T862A; p.V697L.

In a preferred embodiment, the cancer is HER2 NSCLC, comprising a mutation in the tyrosine kinase domain of HER2, in particular a HER2 tyrosine kinase domain activating mutation. Preferably, in this embodiment, zongertinib is administered as first line of therapy. Still preferably, in this embodiment, zongertinib is administered as second or further line of therapy, in particular after a line of therapy comprising administration of an anti-HER2 ADC. Also preferably, in this embodiment, zongertinib is administered as monotherapy.

In a preferred embodiment, the cancer is HER2 NSCLC, comprising a mutation outside of the tyrosine kinase domain of HER2. Preferably, in this embodiment, zongertinib is administered as first line of therapy. Still preferably, in this embodiment, zongertinib is administered as second or further line of therapy. Also preferably, in this embodiment, zongertinib is administered as monotherapy.

In a preferred embodiment, the cancer is unresectable or metastatic non-squamous non-small cell lung cancer (NSCLC) whose tumors have HER2 (ERBB2) tyrosine kinase domain activating mutations and who have received prior systemic therapy. Preferably, in this embodiment, zongertinib is administered as monotherapy.

In an aspect, the cancer is HER2 positive breast cancer, in particular advanced HER2 positive breast cancer or HER2 positive metastatic breast cancer, preferably advanced HER2 positive metastatic breast cancer. Preferably, in this embodiment, zongertinib or the pharmaceutically acceptable salt thereof is administered in combination with a combination partner as described herein as first, second, third or further line of therapy. Also preferably, in this embodiment, the combination partner is selected from the group consisting of trastuzumab deruxtecan, trastuzumab emtansine, trastuzumab, trastuzumab+capecitabine, pertuzumab, trastuzumab+pertuzumab and trastuzumab deruxtecan+pertuzumab.

In an aspect, the cancer is HER2 positive esophageal cancer, HER2 positive gastric cancer, or HER2 positive gastroesophageal junction cancer, in particular HER2 positive esophageal adenocarcinoma, HER2 positive gastric adenocarcinoma, or HER2 positive gastroesophageal junction adenocarcinoma, preferably metastatic HER2 positive esophageal adenocarcinoma, metastatic positive gastric adenocarcinoma, or metastatic HER2 positive gastroesophageal junction adenocarcinoma. Preferably, in this embodiment, zongertinib or the pharmaceutically acceptable salt thereof is administered in combination with a combination partner as described herein as first, second, third or further line of therapy. Also preferably, in this embodiment, the combination partner is trastuzumab deruxtecan.

In another aspect, the cancer is resistant to treatment with the combination partner as defined herein. In another aspect, the cancer is resistant to treatment with the anti-HER2 antibody and/or with the anti-HER2 antibody-drug-conjugate as defined herein.

### Metastasis in the Central Nervous System

As used herein, “cancer comprising at least one metastasis in the central nervous system” and grammatical variants thereof refer to a cancer comprising a primary cancer and a metastatic cancer to the central nervous system. The site of the primary cancer can be as defined herein above. Therefore, the expression “cancer, wherein the cancer comprises at least one metastasis in the central nervous system” is intended as a synonym of and can be replaced with “metastatic cancer, wherein the metastatic cancer comprises or is metastatic cancer to the central nervous system”.

As used herein, “metastasis in the central nervous system” has the meaning commonly attributed to it in the art and in particular refers to a tumor or cancerous lesion in the central nervous system of a patient affected by another primary cancer in the same or in a different location. “Metastasis in the central nervous system” is intended as a synonym of and can be replaced with “lesion in the central nervous system”.

As used herein, “brain metastasis” has the meaning commonly attributed to it in the art and in particular refers to a tumor or cancerous lesion in the brain of a patient affected by another primary cancer in the same or in a different location. “Brain metastasis” is intended as a synonym of and can be replaced with “brain lesion”.

The expressions “at least one metastasis” and “at least one lesion” include patients with one, two, three or more metastases/lesions.

In one aspect, the metastasis or lesion in the central nervous system may be asymptomatic, symptomatic, stable or active. In other words, the metastatic cancer may be asymptomatic, symptomatic, stable or active. In another aspect, the metastasis or lesion in the central nervous system may be asymptomatic or symptomatic. In other words, the metastatic cancer may be asymptomatic or symptomatic.

In another aspect, the metastasis or lesion in the central nervous system may be stable or active. In other words, the metastatic cancer may be stable or active.

In another aspect, the metastasis or lesion in the central nervous system may be asymptomatic or active. In other words, the metastatic cancer may be asymptomatic or active.

In another aspect, the metastasis or lesion in the central nervous system may be symptomatic and/or active. In other words, the metastatic cancer may be symptomatic and/or active.

In another aspect, the metastasis or lesion in the central nervous system may be asymptomatic and/or stable. In other words, the metastatic cancer may be asymptomatic and/or stable.

As used herein, “asymptomatic” has the meaning commonly attributed to it in the art and particularly refers to lesions or metastatic cancers that do not give clinical neurological symptoms. Asymptomatic metastases can be stable, i.e. asymptomatic metastatic cancers can be stable metastatic cancers. “Asymptomatic” and “stable” can be used herein as synonyms.

As used herein, “symptomatic” has the meaning commonly attributed to it in the art and particularly refers to lesions or metastatic cancers that give clinical neurological symptoms. Symptomatic metastases can be active, i.e. symptomatic metastatic cancers can be active metastatic cancers. “Symptomatic” and “active” can be used herein as synonyms.

As used herein, “stable” has the meaning commonly attributed to it in the art and particularly refers to lesions—or to metastatic cancers comprising lesions—that approximately maintain their size/volume. Stable metastases can be asymptomatic, i.e. stable metastatic cancers can be asymptomatic metastatic cancer.

As used herein, “active” has the meaning commonly attributed to it in the art and particularly refers to lesions—or to metastatic cancers comprising lesions—that grow in size/volume. Active metastases can be symptomatic, i.e. active metastatic cancers can be symptomatic metastatic cancer.

Preferably, zongertinib or the pharmaceutically acceptable salt thereof is effective on the at least one metastasis in the central nervous system, in particular in the brain. This means, for example, that zongertinib can have a therapeutic effect on said metastasis, regardless of the therapeutic effect of zongertinib on the primary tumor. Said therapeutic effect can be measured in terms of response in and/or progression of the metastasis. In particular, as used herein, efficacy in a CNS metastasis refers to approximately maintaining or decreasing the size of said metastasis over a period of time between a first assessment (e.g. baseline) and a later assessment (e.g. follow-up). Therefore, zongertinib or the pharmaceutically acceptable salt thereof can be used in the treatment of a metastasis in the central nervous system, in particular in the brain. As used herein, “maintaining or controlling the size/volume of a metastasis” refers to a change in metastasis size (e.g. as measured by its diameter and/or volume) of smaller than −30% to +20% over a period of time between a first assessment (e.g. baseline) and a later assessment (e.g. follow-up).

As used herein, “decreasing or reducing the size/volume of a metastasis” refers to a decrease in metastasis size (e.g. as measured by its diameter and/or volume) of at least −30% over a period of time between a first assessment (e.g. baseline) and a later assessment (e.g. follow-up).

As used herein, “increasing or growing the size/volume of a metastasis” refers to an increase in metastasis size (e.g. as measured by its diameter and/or volume) of at least +20% over a period of time between a first assessment (e.g. baseline) and a later assessment (e.g. follow-up). The size or volume of a metastasis may be measured according to any method known in the art, in particular with the aid of an appropriate imaging technique, for example with computed tomography, positron emission tomography, magnetic resonance and/or as otherwise described in Example 1 below. Metastasis size or volume is preferably determined based on metastasis diameter, as measured by an appropriate imaging technique.

In addition, measurement of efficacy (e.g. response and/or progression in terms of changes in metastasis volume or otherwise) can be performed following standard criteria such as Response Evaluation Criteria in Solid Tumors (RECIST) version 1.1 (or, in brief, RECIST 1.1), the MacDonald criteria and/or Response Assessment in Neuro-Oncology-Brain Metastases (RANO-BM). RECIST 1.1 and RANO-BM criteria are preferred.

As used herein, RECIST 1.1 has the meaning commonly attributed to it in the art and particularly includes the criteria defined in Eisenhauer E A, et al., New response evaluation criteria in solid tumours: Revised RECIST guideline (version 1.1). European Journal of Cancer 2009, 228-247, which is herein incorporated by reference in its entirety.

According to RECIST v1.1, measurable lesions can be defined as those with a longest diameter of at least 10 mm by CT or calliper measurement by clinical exam, except for lymph nodes, which must have a short axis of at least 15 mm when assessed by CT scan. Target lesions can be selected based on size and suitability for reproducible measurement, with up to five lesions total and a maximum of two per organ. The sum of the longest diameters of these target lesions can be calculated at baseline and monitored over time during follow-up.

Tumor response is categorized into four main outcomes:
Complete Response (CR): Disappearance of all target lesions. Any pathological lymph nodes must have reduction in short axis to <10 mm.
Partial Response (PR): At least a 30% decrease in the sum of diameters of target lesions, taking as reference the baseline sum diameters.
Stable Disease (SD): Neither sufficient shrinkage to qualify for PR nor sufficient increase to qualify for PD, taking as reference the smallest sum diameters recorded since treatment initiation.
Progressive Disease (PD): At least a 20% increase in the sum of diameters of target lesions, taking as reference the smallest sum on study (including baseline if that is the smallest), and an absolute increase of at least 5 mm. The appearance of one or more new lesions also indicates progression.

The assessment of CNS metastases by RECIST 1.1 criteria (RECIST-CNS) can refer to a RECIST assessment including lesions in the CNS (especially in the brain) and excluding all other lesions.

As used herein, RANO-BM has the meaning commonly attributed to it in the art and particularly includes guidelines, criteria, methods, systems proposed by RANO working groups to measure CNS (in particular brain) metastases. In particular, RANO-BM includes the criteria defined in Lin N U, et al., Response assessment criteria for brain metastases: proposal from the RANO group. The Lancet 2015, (16) and in Chukwueke Ugonma N, Wen Patrick Y. Use of the Response Assessment in Neuro-Oncology (RANO) criteria in clinical trials and clinical practice. CNS Oncology 2019, 8 (1), CNS28, which are herein incorporated by reference in their entireties.

RANO-BM progression can occur when the sum of the linear measurements exceeds 20% compared with baseline or best response and response can be defined as reduction of the sum of linear measurements by 30% compared with baseline. The patient's clinical status and corticosteroid use are considered. The brain is considered a separate compartment, allowing the status of disease systemically and in the brain to be considered separately, if appropriate. RANO-BM defines measurable disease as lesions of 1 cm or larger, although there is guidance if smaller lesions are allowed. The use of advanced imaging modalities is also recommended by RANO-BM, specifically in assessing radiographic changes which may reflect the effect of treatment. MR perfusion, MR spectroscopy and positron emission tomography (PET) imaging are preferred techniques. RANO-BM criteria are summarized in Table 0 below.

In certain embodiments, “prevention” refers to reducing the risk of or delaying reoccurrence of a CNS metastasis or lesion. Therefore, in an aspect, the invention provides zongertinib or a pharmaceutically acceptable salt thereof for use in the prevention of a cancer comprising at least one metastasis or lesion in the central nervous system, wherein the prevention comprises or consists of delaying or reducing the risk of reoccurrence of a CNS metastasis or lesion. In this aspect, zongertinib, the cancer, the metastasis or lesion, the dosing schedule, the line of administration and/or the pharmaceutical composition can be as defined in any aspect or embodiment described herein. In addition, in this aspect, zongertinib can be administered as monotherapy or in combination with an additional intervention such as surgery and/or radiotherapy and/or a combination partner as defined herein.

### Dosing Schedule

As used herein “dose regime” or “dose schedule” refers to the administration of zongertinib according to any one or more features pertaining to its posology, including but not limited to: daily dose, once/twice daily administration, oral administration, administration as a tablet, administration for a certain duration, administration following another anti-cancer therapy, etc. As used herein, “daily” means within a timeframe of 24 h. The expressions “daily dose” and “total daily dose” refer to the amount of active substance, i.e. zongertinib, which is administered within said timeframe of 24 h. The 24 h timeframe does not necessarily start at noon or midnight. Preferably, the daily dose is administered once or twice per day.

In another aspect, zongertinib or the pharmaceutically acceptable salt thereof is administered daily.

In another aspect, zongertinib or the pharmaceutically acceptable salt thereof is administered orally, preferably as a tablet.

In another aspect, zongertinib or the pharmaceutically acceptable salt thereof is administered daily and orally, preferably as a tablet.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg, preferably of at least 360 mg.

In another aspect, zongertinib or the pharmaceutically acceptable salt thereof is administered in a daily dose of at least 30 mg, preferably of at least 60 mg, preferably of at least 80 mg, preferably of at least 120 mg, preferably of at least 180 mg, preferably of at least 200 mg, preferably of at least 240 mg, preferably of at least 300 mg. Daily doses of at least 120 mg are preferred to achieve efficacy in CNS (in particular brain) metastases and thus treat and/or prevent CNS (in particular brain) metastases. Doses of at least 240 mg are particularly preferred for this purpose.

In another aspect, zongertinib or the pharmaceutically acceptable salt thereof is administered in a daily dose of 30 mg to 600 mg, preferably of 60 mg to 600 mg, preferably of 80 mg to 600 mg, preferably of 120 mg to 600 mg, preferably of 240 mg to 600 mg, preferably of 300 mg to 600 mg.

In another aspect, zongertinib or the pharmaceutically acceptable salt thereof is administered in a daily dose of 30 mg to 360 mg, preferably of 60 mg to 360 mg, preferably of 80 mg to 360 mg, preferably of 120 mg to 360 mg, preferably of 240 mg to 360 mg, preferably of 300 mg to 360 mg.

In another aspect, zongertinib or the pharmaceutically acceptable salt thereof is administered in a daily dose of 30 mg, 60 mg, 120 mg, 180 mg, 200 mg, 240 mg, 300 mg, 360 mg, 400 mg, 420 mg, 480 mg, 500 mg, 540 mg or 600 mg.

In another aspect, zongertinib or the pharmaceutically acceptable salt thereof is administered in a daily dose of 30 mg, 60 mg, 120 mg, 180 mg, 200 mg, 240 mg, 300 mg or 360 mg.

In another aspect, zongertinib or the pharmaceutically acceptable salt thereof is administered in a daily dose of 120 mg, 180 mg, 200 mg, 240 mg, 300 mg or 360 mg.

In preferred embodiments, zongertinib or the pharmaceutically acceptable salt thereof is administered in a daily dose of 120 mg or 240 mg.

In another aspect, zongertinib or the pharmaceutically acceptable salt thereof is administered in a daily dose of 120 mg.

In another aspect, zongertinib or the pharmaceutically acceptable salt thereof is administered in a daily dose of 180 mg.

In another aspect, zongertinib or the pharmaceutically acceptable salt thereof is administered in a daily dose of 200 mg.

In another aspect, zongertinib or the pharmaceutically acceptable salt thereof is administered in a daily dose of 240 mg.

In another aspect, zongertinib or the pharmaceutically acceptable salt thereof is administered in a daily dose of 300 mg.

In another aspect, zongertinib or the pharmaceutically acceptable salt thereof is administered in a daily dose of 360 mg.

Within a preferred embodiment, zongertinib or the pharmaceutically acceptable salt thereof is administered once or twice daily. This means, that the daily dose, in particular the daily dose as defined in any aspect described herein, is administered either as a single dose (once daily) or is divided in two separate administrations, each administered at a different time point of the day (twice daily), i.e. there are two administrations within 24 h. When zongertinib is administered twice daily, the two separate administrations are preferably separated by a time interval of at least 1, 2, 3, 4, 5, 6, 7, 8, 9, 10, 11 or 12 hours, preferably of 4, 5, 6, 7, 8, 9, 10, 11 or 12 hours, still preferably of 8, 9, 10, 11 or 12 hours, also preferably of approximately 12 hours. In case of a twice daily administration, it is preferred that each of the two daily administrations of zongertinib corresponds to half the daily dose. When zongertinib is administered twice daily, the two separate administrations are preferably separated by a time interval of at least 1, 2, 3, 4, 5, 6, 7, 8, 9, 10, 11 or 12 hours, preferably of 4, 5, 6, 7, 8, 9, 10, 11 or 12 hours, still preferably of 8, 9, 10, 11 or 12 hours, also preferably of approximately 12 hours.

In a preferred embodiment, zongertinib or the pharmaceutically acceptable salt thereof is administered once daily. In a preferred embodiment, zongertinib or the pharmaceutically acceptable salt thereof is administered as a single dose every 24 h.

In a preferred embodiment, zongertinib or the pharmaceutically acceptable salt thereof is administered twice daily. In a preferred embodiment, zongertinib or the pharmaceutically acceptable salt thereof is administered twice every 24 h.

In another aspect, zongertinib or the pharmaceutically acceptable salt thereof is administered at least for 21 consecutive days. In further preferred embodiments, zongertinib or the pharmaceutically acceptable salt thereof is administered for 21 days multiplied by X, wherein X is a natural number equal or larger than 1. As used herein, “21 days” and “3 weeks” are intended as synonyms. It is also possible, that in the overall cancer treatment between the treatment times including administering zongertinib a dose-free time-interval is included.

In another aspect, zongertinib or the pharmaceutically acceptable salt thereof is administered once daily in a daily dose of 60 mg, 120 mg, 180 mg, 240 mg, 300 mg, 360 mg, 400 mg, 420 mg, 480 mg, 500 mg, 540 mg or 600 mg or zongertinib or the pharmaceutically acceptable salt thereof is administered twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered once daily in a daily dose of 60 mg, 120 mg, 180 mg, 240 mg, 300 mg or 360 mg or zongertinib is administered twice daily in a daily dose of 30 mg, 60 mg, 120 mg, 200 mg, 300 mg or 360 mg.

In another aspect, zongertinib or the pharmaceutically acceptable salt thereof is administered once daily in a daily dose of 120 mg, 180 mg, 240 mg, 300 mg or 360 mg or zongertinib is administered twice daily in a daily dose of 120 mg, 200 mg, 300 mg or 360 mg.

In another aspect, zongertinib or the pharmaceutically acceptable salt thereof is administered once daily in a daily dose of 60 mg, 120 mg, 180 mg, 240 mg, 300 mg, 360 mg, 400 mg, 420 mg, 480 mg, 500 mg, 540 mg or 600 mg.

In another aspect, zongertinib or the pharmaceutically acceptable salt thereof is administered once daily in a daily dose of 60 mg, 120 mg, 180 mg, 240 mg, 300 mg or 360 mg.

In another aspect, zongertinib or the pharmaceutically acceptable salt thereof is administered once daily in a daily dose of 120 mg, 180 mg, 240 mg, 300 mg or 360 mg.

In another aspect, zongertinib or the pharmaceutically acceptable salt thereof is administered once daily in a daily dose of 120 mg or 240 mg.

In another aspect, zongertinib or the pharmaceutically acceptable salt thereof is administered once daily in a daily dose of 120 mg.

In another aspect, zongertinib or the pharmaceutically acceptable salt thereof is administered once daily in a daily dose of 180 mg.

In another aspect, zongertinib or the pharmaceutically acceptable salt thereof is administered once daily in a daily dose of 200 mg.

In another aspect, zongertinib or the pharmaceutically acceptable salt thereof is administered once daily in a daily dose of 240 mg.

In another aspect, zongertinib or the pharmaceutically acceptable salt thereof is administered once daily in a daily dose of 300 mg.

In another aspect, zongertinib or the pharmaceutically acceptable salt thereof is administered once daily in a daily dose of 360 mg.

In another aspect, zongertinib or the pharmaceutically acceptable salt thereof is administered twice daily in a daily dose of 30 mg, 60 mg, 120 mg, 200 mg, 300 mg, 360 mg, 400 mg, 420 mg, 480 mg, 500 mg, 540 mg or 600 mg.

In another aspect, zongertinib or the pharmaceutically acceptable salt thereof is administered twice daily in a daily dose of 30 mg, 60 mg, 120 mg, 200 mg, 300 mg or 360 mg. In another aspect, zongertinib or the pharmaceutically acceptable salt thereof is administered twice daily in a daily dose of 120 mg, 200 mg, 300 mg or 360 mg.

In another aspect, zongertinib or the pharmaceutically acceptable salt thereof is administered twice daily in a daily dose of 120 mg. Preferably, in this aspect, each of the two daily administrations is of 60 mg.

In another aspect, zongertinib or the pharmaceutically acceptable salt thereof is administered twice daily in a daily dose of 200 mg. Preferably, in this aspect, each of the two daily administrations is of 100 mg.

In another aspect, zongertinib or the pharmaceutically acceptable salt thereof is administered twice daily in a daily dose of 300 mg. Preferably, in this aspect, each of the two daily administrations is of 150 mg.

In another aspect, zongertinib or the pharmaceutically acceptable salt thereof is administered twice daily in a daily dose of 360 mg. Preferably, in this aspect, each of the two daily administrations is of 180 mg.

In any of the aspects defined herein, zongertinib or the pharmaceutically acceptable salt thereof preferably is administered orally, in particular as a tablet.

It is also possible to combine any of the above defined doses of zongertinib or the pharmaceutically acceptable salt thereof during the course of treatment, for example by altering the daily dose. For instance, it is possible to increase or decrease a dose of 120 mg (once or twice daily) for subsequent administrations. Dosing schedules where the initial dose is increased to achieve efficacy in CNS (in particular brain) metastases and thus treat and/or prevent CNS (in particular brain) metastases are preferred.

The dose regimes described in this paragraph are also applicable in cases, wherein the patient already received one or more systemic anti-cancer treatments or therapies.

### Second or Further Line Administration

In an aspect, zongertinib or the pharmaceutically acceptable salt thereof is administered as a first line therapy. In this aspect, zongertinib, the cancer, the metastasis or lesion, the dosing schedule and/or the pharmaceutical composition can be as defined in any aspect or embodiment described herein. In addition, in this aspect, zongertinib can be administered as monotherapy or in combination with an additional intervention such as surgery and/or radiotherapy and/or a combination partner.

As used herein, “first line” and grammatical variants thereof have the meaning known in the art. In particular, “first line” and grammatical variants thereof can refer to the administration of zongertinib or the pharmaceutically acceptable salt thereof as the initial treatment for the management and/or treatment of cancer. First line therapy may encompass systemic, surgical and/or radiation-based interventions. The designation of a therapy as “first line” does not preclude its use in combination with other modalities in the first line or its sequential administration as part of a multimodal treatment strategy in a second or further line. First line therapy may be delivered in different temporal contexts relative to surgery, if performed, e.g. as neoadjuvant therapy or as adjuvant therapy.

In another aspect, zongertinib or the pharmaceutically acceptable salt thereof is administered as a second or further line therapy. In this aspect, zongertinib, the cancer, the metastasis or lesion, the dosing schedule and/or the pharmaceutical composition can be as defined in any aspect or embodiment described herein. In addition, in this aspect, zongertinib can be administered as monotherapy or in combination with an additional intervention such as surgery and/or radiotherapy and/or a combination partner.

As used herein, “second or further line” and grammatical variants thereof have the meaning known in the art. In particular, “second or further line” and grammatical variants thereof can refer to the administration of zongertinib or the pharmaceutically acceptable salt thereof after or following a first or prior line of therapy that has failed, stopped working, decreased efficacy, intolerable side effects or been only partially successful, according to the judgment of the attending physician, especially wherein the first or prior line of therapy as such is never again administered to the patient. Each line of therapy may include the administration of one or more agents. The expression “second or further line” can be read as “second line or further line”. The expression “first or previous line” can be read as “first line or previous line”.

In another aspect, zongertinib or the pharmaceutically acceptable salt thereof is administered as a second or further line therapy, after or following a first or previous line therapy. For clarity, the latter sentence is intended to include the following two aspects:
zongertinib or the pharmaceutically acceptable salt thereof is administered as a second line therapy, after or following a first line therapy;
zongertinib or the pharmaceutically acceptable salt is administered as a further line therapy, after or following a previous line therapy.

In this context, the terms “after” and “following” mean that the first or previous line therapy is administered during a first time period, for example over the course of a few hours, days or one or more weeks, using one or more doses, and is followed by administration of zongertinib or the pharmaceutically acceptable salt thereof during a second time period, for example over the course of a few hours, days or one or more weeks, using one or more doses, provided there is no overlap between the first and second time periods. In particular, when zongertinib or the pharmaceutically acceptable salt thereof is administered as second or further line therapy, it is preferred that the first or previous line therapy and zongertinib, or the pharmaceutically acceptable salt thereof, are not administered on the same day. Also in particular, when zongertinib or the pharmaceutically acceptable salt thereof is administered as second or further line therapy, the agents(s) administered in the first or previous line therapy is/are not re-started with the same dose once zongertinib, or the pharmaceutically acceptable salt thereof, is administered. The terms “after” and “following” do not require that zongertinib or the pharmaceutically acceptable salt thereof is administered directly after or directly following the first or previous line of therapy. Therefore, when zongertinib or the pharmaceutically acceptable salt thereof is administered as second or further line therapy, it may be that another line of therapy is administered between the first or previous line and zongertinib or the pharmaceutically acceptable salt thereof.

The skilled person will recognize that the terms “before”, “prior”, “after” and “following” are used herein to refer to different and/or separate lines of therapy. In other words, any reference to the administration of zongertinib or the pharmaceutically acceptable salt thereof after or following a first or previous line of therapy corresponds to a reference to the administration of zongertinib or the pharmaceutically acceptable salt thereof as second or further line of therapy after administration, even when this is not explicitly stated.

In another aspect, zongertinib or the pharmaceutically acceptable salt is administered as a second or further line therapy, after or following a first or previous line therapy, wherein the first or previous line therapy comprises or consists of administration of standard of care, optionally followed by maintenance therapy. Depending on the cancer that the patient is suffering from, said standard of care therapy may comprise a combination of pembrolizumab, pemetrexed and platinum-based chemotherapy, wherein said combination is optionally administered once every 3 weeks for one or more administrations, preferably for 1, 2, 3 or 4 administrations, more preferably for 4 administrations. Depending on the cancer that the patient is suffering from, said maintenance therapy may comprise a combination of pembrolizumab and pemetrexed, optionally administered once every 3 weeks for one or more administrations. In another aspect, zongertinib or the pharmaceutically acceptable salt is administered as a second or further line therapy, after or following a first or previous line therapy, wherein the first or previous line therapy comprises or consists of administration of chemotherapy. As used herein, the term “chemotherapy” has the meaning commonly attributed to it in the art and thus can refer to a chemical agent or a combination of chemical agents useful in the prevention and/or treatment of cancer, especially where the agent or combination is or comprises a cytotoxic and/or cytostatic agent. For example, the first or previous line chemotherapy can be selected from chemotherapeutic agents defined hereinbelow in the context of pharmacologically active substances that may be administered in combination with zongertinib, or a pharmaceutically acceptable salt thereof, e.g. combination partners. Preferably, chemotherapy is administered systemically, i.e. it is systemic chemotherapy.

In another aspect, zongertinib or the pharmaceutically acceptable salt is administered as a second or further line therapy, after or following a first or previous line therapy, wherein the first or previous line therapy comprises or consists of administration of systemic anti-cancer therapy.

As used herein, the term “systemic anti-cancer therapy” includes at least one systemic anti-cancer therapy agent, alone or in combination with another drug compound or active ingredient. Systemic anti-cancer therapy agents may include but are not limited to: small or large chemical molecules, metal complexes for example comprising platinum (Pt), biologics and combinations thereof. For example, the first or previous line systemic anti-cancer therapy can be selected from systemic anti-cancer therapy agents defined hereinbelow in the context of pharmacologically active substances that may be administered in combination with zongertinib, or a pharmaceutically acceptable salt thereof, e.g. combination partners.

As used herein, the term “systemic” has the meaning commonly attributed to it in the art and thus can refer to a route of administration affecting the entire body, as opposed to local administration where the effect is generally local. Systemic administration can be enteral (e.g. via the gastrointestinal tract) or parenteral (e.g. via injection, infusion, implantation, etc.). Preferably, said systemic anti-cancer therapy is parenteral anti-cancer therapy.

In an embodiment, zongertinib or the pharmaceutically acceptable salt thereof is administered as second or further line therapy and the first or previous line of therapy comprises administration of at least one agent selected from the group consisting of: platinum-based chemotherapy, anti-HER2 conjugates, antibody-drug taxanes, anti-metabolites, immunotherapeutic agents and combinations thereof. Preferably, the term “combinations thereof” might include the separate administration of two or more members of that list in different time intervals or at different points in time, as different lines of therapy or the administration of two or more members of that list in combination in the same line of therapy. The combination might for instance include a first line treatment including a platinum-based chemotherapy agent and, later on, a second line treatment based on an ADC, preferably an anti-HER2-ADC, or vice versa, prior to a treatment based on zongertinib. The combination can be a planned sequence or can be performed as a function of the treatment outcome.

As used herein, the expression “platinum-based chemotherapy” and grammatical variants thereof have the meaning commonly attributed to them in the art and thus can refer to one or more chemotherapy agents comprising platinum (Pt). Preferred platinum-based chemotherapy agents include oxaliplatin, carboplatin and/or cisplatin. Still preferred platinum-based chemotherapy agents include carboplatin and/or cisplatin.

The term “antibody drug conjugate” (also abbreviated herein as “ADC”), as used herein, is well known in the art and describes a group of therapeutics that combine the specificity of tumor-targeting binders, such as e.g. antibodies, with the potency of highly cytotoxic agents. Accordingly, such conjugates of cytotoxic and/or cytostatic agents to antibodies specific for tumor cells are powerful tools to specifically target cancer cells for their destruction. ADCs are well known in the art and have been reviewed, for example, in Dumontet et al. 2023 (Dumontet, C., Reichert, J. M., Senter, P. D. et al. Nat Rev Drug Discov 22, 641-661 (2023)).

In particular, “anti-HER2 antibody-drug conjugate”, as used herein, refers to an ADC whose tumor-targeting binder is an antibody directed to, targeting and/or binding HER2, such as the anti-HER2 antibodies defined herein.

The expressions “anti-HER2 antibody-drug conjugate” and “anti-HER2 ADC” thus refer to an anti-HER2 antibody conjugated to a cytotoxic drug, also called payload, optionally via a linker. The anti-HER2 antibody in the ADC can deliver the payload to cells expressing HER2, especially to cells with high levels of HER2.

Preferably, “anti-HER2 antibody-drug conjugate” as used herein is as defined in any of the aspects or embodiments of WO 2025/132522, which is herein incorporated by reference in its entirety.

In an aspect, the anti-HER2 antibody-drug conjugate comprises an anti-HER2 antibody that is an inhibitory antibody.

In another aspect, the anti-HER2 antibody-drug conjugate comprises an anti-HER2 antibody binding specifically to HER2.

In another aspect, the anti-HER2 antibody-drug conjugate comprises an anti-HER2 antibody binding specifically to the extracellular domain of HER2.

In another aspect, the anti-HER2 antibody-drug conjugate comprises an anti-HER2 antibody that is an inhibitory antibody binding specifically to HER2.

In another aspect, the anti-HER2 antibody-drug conjugate comprises an anti-HER2 antibody binding specifically to an extracellular domain of HER2.

In another aspect, the anti-HER2 antibody-drug conjugate comprises an anti-HER2 antibody selected from the group consisting of trastuzumab, pertuzumab, margetuximab, zanidatamab, disitamab, anvatabart, hersintuzumab and anbenitamab. In this aspect, trastuzumab, pertuzumab, margetuximab, zanidatamab, hersintuzumab and anbenitamab can be as defined below in any of the aspects concerning the anti-HER2 antibody.

In another aspect, the anti-HER2 antibody-drug conjugate comprises trastuzumab. In this aspect, trastuzumab can be as defined herein, e.g. in any of the aspects concerning the anti-HER2 antibody.

In another aspect, the anti-HER2 antibody-drug conjugate is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

In another aspect, the anti-HER2 antibody-drug conjugate is selected from the group consisting of trastuzumab deruxtecan and trastuzumab emtansine. In this aspect, trastuzumab can be as defined herein, e.g. in any of the aspects concerning the anti-HER2 antibody.

In another aspect, the anti-HER2 antibody-drug conjugate is trastuzumab deruxtecan.

In another aspect, the anti-HER2 antibody-drug conjugate is trastuzumab emtansine.

As used herein in any embodiment, “trastuzumab deruxtecan”, “trastuzumab emtansine”, “trastuzumab rezetecan”, “trastuzumab duocarmazine”, “zanidatamab zovodotin”, “disitamab vedotin” and “anvatabart opadotin” refer to the respective ADCs commonly known in the art with those names, in particular as further defined hereinbelow. The additional explanations hereinbelow are provided merely for the sake of clarity and are not intended to be limiting.

The term “trastuzumab emtansine” or “ado-trastuzumab emtansine”, also known as T-DM1 or Kadcyla, refers to the antibody-drug conjugate comprising the anti-HER2 antibody trastuzumab, a thioether linker, and the microtubule inhibitor emtansine or DM1—a derivative of maytansine—as cytotoxic payload. The International Nonproprietary Name (INN) “trastuzumab emtansine” is published in the WHO's INN Recommended List 65, which is herein incorporated by reference.

The term “trastuzumab deruxtecan”, also known as T-DXd or Enhertu, refers to the antibody-drug conjugate comprising the anti-HER2 antibody trastuzumab, a linker, and the topoisomerase I inhibitor deruxtecan or DXd—a derivative of exatecan—as cytotoxic payload. The International Nonproprietary Name (INN) “trastuzumab deruxtecan” is published in the WHO's INN Recommended List 78, which is herein incorporated by reference.

Trastuzumab rezetecan, also known as SHR-A1811, comprises the anti-HER2 antibody trastuzumab conjugated to a DNA topoisomerase I inhibitor (SHR169265) via cleavable tetrapeptide-based linkers.

Trastuzumab duocarmazine or [vic-]trastuzumab duocarmazine, also known as SYD985, comprises the anti-HER2 antibody trastuzumab and a cleavable linker-drug called valine-citrulline-seco-DUocarmycin-hydroxyBenzamide-Azaindole (vc-seco-DUBA).

Zanidatamab zovodotin, also known as ZW49, comprises the anti-HER2 bispecific/biparatopic antibody zanidatamab, a protease cleavable linker and the microtubule inhibitor auristatin at a drug-to-antibody ratio (DAR) of 2.

Disitamab vedotin, also known as Aidixi or RC48, comprises the anti-HER2 antibody disitamab (also known as hertuzumab), a histone cleavable linker and vedotin, i.e. monomethyl auristatin E (MMAE).

Anvatabart opadotin, also known as ARX788, comprises an anti-HER2 antibody conjugated to a noncleavable Amberstatin (AS269) drug-linker with a DAR of 1.9.

The INNs as used herein are meant to also encompass all biosimilar molecules with the same structure, in particular comprising the same, or substantially the same, amino acid sequences as the originator antibody, including but not limited to those biosimilar antibodies authorized under 42 USC § 262 subsection (k) in the US and equivalent regulations in other jurisdictions. A “biosimilar” as used herein refers to an antibody (isolated or as part of and ADC) or antigen-binding fragment that has the same primary amino acid sequence as compared to a reference antibody (e.g., trastuzumab) and optionally, may have detectable differences in post-translation modifications (e.g., glycosylation and/or phosphorylation) as compared to the reference antibody (e.g., a different glycoform).

As used herein, the term “taxane” and grammatical variants thereof have the meaning commonly attributed to them in the art and thus can refer to a diterpene with a taxadiene core. Taxanes usually work as mitotic inhibitors, specifically as disruptors of the microtubule function. Preferred taxanes include cabazitaxel, larotaxel, tesetaxel, docetaxel and/or paclitaxel. Still preferred taxanes include docetaxel and/or paclitaxel.

As used herein, the term “anti-metabolite” and grammatical variants thereof have the meaning commonly attributed to them in the art and thus can refer to one or more agents that interfere with metabolic processes within cells. Specifically, anti-metabolites can inhibit a metabolite. Preferred anti-metabolites include: 5-fluorouracil, 6-mercaptopurine, capecitabine, cytarabine, floxuridine, fludarabine, hydroxycarbamide, methotrexate, phototrexate, gemcitabine, pemetrexed and/or tegafur. Still preferred anti-metabolites include: gemcitabine, pemetrexed and/or tegafur. For example, the first or previous line anti-metabolite can be selected from antimetabolites defined hereinbelow in the context of pharmacologically active substances that may be administered in combination with zongertinib, or a pharmaceutically acceptable salt thereof, e.g. combination partners.

As used herein, the terms “immunotherapy”, “immunotherapeutic agent” and grammatical variants thereof have the meaning commonly attributed to them in the art and thus can refer to one or more agents that interfere with (e.g. activate or suppress) the immune system and thereby contribute to the prevention and/or treatment of cancer. Preferred immunotherapeutic agents include: cemiplimab, dostarlimab, trastuzumab, pertuzumab, margetuximab, pembrolizumab, durvalumab, atezolizumab, nivolumab, ipilimumab, tremelimumab, and/or ramucirumab. Still preferred immunotherapeutic agents include: pembrolizumab, durvalumab, atezolizumab, nivolumab, ipilimumab, tremelimumab, and/or ramucirumab. For example, the first or previous line immunotherapy can be selected from immunotherapie agents defined hereinbelow in the context of pharmacologically active substances that may be administered in combination with zongertinib, or a pharmaceutically acceptable salt thereof, e.g. combination partners.

In an embodiment, zongertinib or the pharmaceutically acceptable salt thereof is administered as second or further line therapy and the first or previous line of therapy comprises administration of at least one agent selected from the group consisting of: carboplatin, cisplatin, trastuzumab deruxtecan, trastuzumab emtansine, pemetrexed, docetaxel, paclitaxel, gemcitabine, pembrolizumab, durvalumab, tremelimumab, ramucirumab, atezolizumab, tegafur, nivolumab, ipilimumab and combinations thereof.

In an embodiment, zongertinib or the pharmaceutically acceptable salt thereof is administered as second or further line therapy and the first or previous line of therapy comprises administration of at least one agent selected from the group consisting of: carboplatin, cisplatin, trastuzumab deruxtecan, trastuzumab emtansine, pemetrexed, docetaxel, paclitaxel, gemcitabine, pembrolizumab, durvalumab, tremelimumab, ramucirumab, atezolizumab, tegafur, nivolumab and ipilimumab.

In an embodiment, zongertinib or the pharmaceutically acceptable salt thereof is administered as second or further line therapy and the first or previous line of therapy comprises administration of a platinum-based chemotherapy.

In an embodiment, zongertinib or the pharmaceutically acceptable salt thereof is administered as second or further line therapy and the first or previous line of therapy comprises administration of pembrolizumab, pemetrexed and/or platinum-based chemotherapy.

In an embodiment, zongertinib or the pharmaceutically acceptable salt thereof is administered as second or further line therapy and the first or previous line of therapy comprises administration of pembrolizumab, pemetrexed and platinum-based chemotherapy.

In an embodiment, zongertinib or the pharmaceutically acceptable salt thereof is administered as second or further line therapy and the first or previous line of therapy comprises administration of pembrolizumab, pemetrexed and cisplatin.

In an embodiment, zongertinib or the pharmaceutically acceptable salt thereof is administered as second or further line therapy and the first or previous line of therapy comprises administration of pembrolizumab, pemetrexed and carboplatin.

In an embodiment, zongertinib or the pharmaceutically acceptable salt thereof is administered as second or further line therapy and the first or previous line of therapy comprises administration of pembrolizumab and/or pemetrexed.

In another aspect, zongertinib or the pharmaceutically acceptable salt thereof is administered at least 21 days after the last day of the first or previous line of therapy.

### Additional Intervention

In the treatment of patients with at least one metastasis in the central nervous system or in the prevention thereof, as described herein, zongertinib, or the pharmaceutically acceptable salt thereof may be administered in combination with additional intervention selected from the group consisting of radiation, surgery, an additional therapeutic agent such as chemotherapy and/or a combination partner as defined below, and a combination thereof. In aspects referring to a combination of zongertinib or the pharmaceutically acceptable salt thereof with an additional intervention, in particular surgery and/or radiotherapy and/or a combination partner, zongertinib, the (metastatic) cancer, the metastasis or lesion, the dosing schedule, the line of administration and/or the pharmaceutical composition can be as defined in any aspect or embodiment described herein.

In one aspect, the present invention relates to zongertinib, or a pharmaceutically acceptable salt thereof, for use in the prevention and/or treatment of cancer, wherein the cancer comprises at least one metastasis or lesion in the central nervous system as described herein, wherein zongertinib or the pharmaceutically acceptable salt thereof is administered in combination with a cytostatic and/or cytotoxic active substance and/or in combination with radiotherapy and/or in combination with surgery and/or in combination with chemotherapy and/or in combination with immunotherapy.

In this context, “combination” or “combined” and grammatical variants thereof within the meaning of this invention include, but are not limited to, a product, product for use, use or method that results from the mixing or combining of more than one therapeutic intervention, in particular zongertinib and the combination partner as defined herein. In one aspect, zongertinib or the pharmaceutically acceptable salt thereof is administered in combination with a combination partner. The expressions “combination”, “combined” and grammatical variants thereof comprise both fixed (e.g. pharmaceutical composition in one dosage form) and non-fixed (e.g. free) combinations (e.g. kits), products, products for use, uses and methods, such as e.g. the simultaneous, concurrent, sequential, successive, alternate or separate use of zongertinib and the combination partner as defined herein.

As used herein, “combination” or “combined” and grammatical variants thereof refer to a combination that takes place within the same line of treatment.

The term “simultaneous” refers to the administration of both compounds/compositions at substantially the same time. This form of administration may also be referred to as “concomitant” administration. The term “concurrent” refers to administration of the active ingredients within the same general time period, for example on the same day(s) but not necessarily at the same time. The term “sequential” administration includes administration of one active ingredient during a first time period, for example over the course of a few hours, days or a week, using one or more doses, followed by administration of the other active ingredient during a second time period, for example over the course of a few hours, days or a week, using one or more doses. An overlapping schedule may also be employed, which includes administration of the active ingredients on different days over the treatment period, not necessarily according to a regular sequence. The term “successive” administration, alternatively, refers to an administration where the second administration step is carried out immediately once the administration of the first compounds has been finished. Alternate administration includes administration of one active ingredient during a time period, for example over the course of a few hours, days or a week, followed by administration of the other active ingredient during a subsequent period of time, for example over the course of a few hours, days or a week, and then repeating the pattern for one or more cycles, wherein the overall number of repeats depends on the chosen dosage regimen. Variations of these general administration forms may also be employed.

Accordingly, in another aspect, zongertinib or the pharmaceutically acceptable salt thereof and the combination partner are administered simultaneously, concurrently, sequentially, successively, alternately or separately.

Remarkably, radiotherapy is not required to achieve efficacy in the CNS and thus treat and/or prevent CNS metastases with the administration of zongertinib or a pharmaceutically acceptable salt thereof. Nevertheless, for the treatment of diseases of oncological nature, anticancer agents may be combined with radiotherapy, e.g. irradiation treatment, and/or surgery. Zongertinib, or a pharmaceutically acceptable salt thereof, for use as described herein, can be used in combination with radiotherapy, in particular to the CNS, especially to the brain. For example, a cancer patient with CNS metastases may receive radiotherapy before and/or after and/or at the same time (including concomitantly, simultaneously, sequentially, concurrently, etc) with receiving therapy with zongertinib, or a pharmaceutically acceptable salt thereof, as described herein. Therefore, in an aspect, the invention provides zongertinib or a pharmaceutically acceptable salt thereof for use in the prevention and/or treatment of cancer in combination with radiotherapy to the central nervous system (especially the brain), wherein the cancer comprises at least one metastasis or lesion in the central nervous system (especially in the brain). In this aspect, zongertinib, the cancer, the metastasis or lesion, the dosing schedule, the line of administration and/or the pharmaceutical composition can be as defined in any aspect or embodiment described herein. In addition, in this aspect, zongertinib can be additionally combined with surgery and/or a combination partner.

In embodiments, zongertinib, or a pharmaceutically acceptable salt thereof, for use as described herein, can be used as adjuvant therapy in combination with a surgical procedure. Zongertinib or the pharmaceutically acceptable salt thereof may be administered for the purpose of diminishing the size of a tumor before surgical procedure (referred to as pre-operative adjuvant chemotherapy or neoadjuvant therapy), or may be administered after a surgical procedure for the purpose of reducing/eliminating any remaining cancer cells and/or preventing/delaying/reducing the risk of reoccurrence. Therefore, in an aspect, the invention provides zongertinib or a pharmaceutically acceptable salt thereof for use in the prevention and/or treatment of cancer, wherein the cancer comprises at least one metastasis or lesion in the central nervous system, wherein zongertinib or the pharmaceutically acceptable salt thereof is administered in combination with surgical removal of the metastasis or lesion. In this aspect, zongertinib, the cancer, the metastasis or lesion, the dosing schedule, the line of administration and/or the pharmaceutical composition can be as defined in any aspect or embodiment described herein. In addition, in this aspect, zongertinib can be additionally combined with radiotherapy and/or a combination partner.

In embodiments, zongertinib, or a pharmaceutically acceptable salt thereof, for use as described herein, may be administered in combination with a cytostatic and/or cytotoxic active substance and/or in combination with immunotherapy.

In embodiments, zongertinib, or a pharmaceutically acceptable salt thereof, for use as described herein, may be used in combination with one or several other pharmacologically active substances such as state-of-the-art or standard-of-care compounds, such as e.g. cell proliferation inhibitors, anti-angiogenic substances, steroids or immune modulators/checkpoint inhibitors, and the like.

Pharmacologically active substances which may be administered in combination with zongertinib, or a pharmaceutically acceptable salt thereof, i.e. combination partners as defined herein, include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrozole, liarozole, vorozole, exemestane, atamestane), LHRH agonists and antagonists (e.g. goserelin acetate, luprolide), inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors), inhibitors are for example (anti-) growth factor antibodies, (anti-) growth factor receptor antibodies and tyrosine kinase inhibitors, such as for example cetuximab, gefitinib, afatinib, nintedanib, imatinib, lapatinib, bosutinib, bevacizumab, pertuzumab and trastuzumab); anti-HER2 antibodies; anti-HER2 antibody drug conjugates (e.g. trastuzumab emtansine, trastuzumab deruxtecan); antimetabolites (e.g. antifolates such as methotrexate, raltitrexed, pyrimidine analogues such as 5fluorouracil (5fluorineU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); antitumor antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); angiogenesis inhibitors (e.g. tasquinimod), tubuline inhibitors; DNA synthesis inhibitors, PARP inhibitors, topoisomerase inhibitors (e.g. epipodophyllotoxins such as for example etoposide and etopophos, teniposide, amsacrin, topotecan, irinotecan, mitoxantrone), serine/threonine kinase inhibitors (e.g. PDK 1 inhibitors, Raf inhibitors, A-Raf inhibitors, B-Raf inhibitors, C-Raf inhibitors, mTOR inhibitors, mTORC1/2 inhibitors, PI3K inhibitors, PI3Kα inhibitors, dual mTOR/PI3K inhibitors, STK 33 inhibitors, AKT inhibitors, PLK 1 inhibitors, inhibitors of CDKs, Aurora kinase inhibitors), tyrosine kinase inhibitors (e.g. PTK2/FAK inhibitors), protein protein interaction inhibitors (e.g. IAP activator, Mcl-1, MDM2/MDMX), MEK inhibitors, ERK inhibitors, KRAS inhibitors (e.g. KRAS G12C inhibitors), signalling pathway inhibitors (e.g. SOS1 inhibitors), FLT3 inhibitors, BRD4 inhibitors, IGF-1R inhibitors, TRAILR2 agonists, Bcl-xL inhibitors, Bcl-2 inhibitors, Bcl-2/Bcl-xL inhibitors, ErbB receptor inhibitors, BCR-ABL inhibitors, ABL inhibitors, Src inhibitors, rapamycin analogs (e.g. everolimus, temsirolimus, ridaforolimus, sirolimus), androgen synthesis inhibitors, androgen receptor inhibitors, DNMT inhibitors, HDAC inhibitors, ANG1/2 inhibitors, CYP17 inhibitors, radiopharmaceuticals, proteasome inhibitors, immunotherapeutic agents such as immune checkpoint inhibitors (e.g. CTLA4, PD1, PD-L1, PD-L2, LAG3, and TIM3 binding molecules/immunoglobulins, such as e.g. ipilimumab, nivolumab, pembrolizumab), ADCC (antibody-dependent cell-mediated cytotoxicity) enhancers (e.g. anti-CD33 antibodies, anti-CD37 antibodies, anti-CD20 antibodies), T-cell engagers (e.g. bi-specific T-cell engagers (BiTEs®) like e.g. CD3×BCMA, CD3×CD33, CD3×CD19), PSMA×CD3), tumor vaccines and various chemotherapeutic agents such as amifostin, anagrelid, clodronat, filgrastin, interferon, interferon alpha, leucovorin, procarbazine, levamisole, mesna, mitotane, pamidronate and porfimer.

Particularly preferred combination partners are selected from the group consisting of an anti-HER2 antibody, an anti-HER2 antibody-drug conjugate, an anti-metabolite and a combination thereof. In the context of the previous sentence, “a combination thereof” includes a combination of anti-HER2 antibodies (such as trastuzumab+pertuzumab), a combination of anti-HER2 antibody-drug conjugates (such as T-DXd+T-DM1), a combination of anti-metabolites, a combination of at least one anti-HER2 antibody with at least one anti-HER2 antibody-drug conjugate (such as pertuzumab+T-DXd), a combination of at least one anti-HER2 antibody with at least one anti-metabolite (such as trastuzumab+capecitabine) and a combination of at least one anti-HER2 antibody-drug conjugate with at least one anti-metabolite. The use of the singular “a” or “the” combination partner does not preclude that a combination thereof as defined herein may be used. Preferably, “combination partner” is an anti-HER2 antibody or an anti-HER2 antibody-drug conjugate. Therefore, the anti-HER2 antibody or anti-HER2 antibody-drug conjugate as defined herein can be referred to individually as “combination partner” or collectively as “combination partners”.

In an aspect, the combination partner is an anti-HER2 antibody.

The term “antibody” which may be used interchangeably with “antibody molecule” encompasses various antibody structures, including but not limited to poly- or monoclonal, chimeric, humanized, human, mono-, bi- or multispecific antibodies, single chain antibodies, single domain antibodies, and fragmented antibodies (also referred to as antibody fragments), such as Fab, F (ab) 2, F(ab′) 2, Fab′, single chain variable-fragments (scFv) or antigen binding domains of an antibody, so long as they exhibit the desired antigen-binding activity. The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. The term further encompasses a fragment of an immunoglobulin, like Fab fragments and polypeptides having one or more variable domains derived from an immunoglobulin, like single chain antibodies (scFv), single domain antibodies, and the like.

The term antibody “fragment” shall refer to fragments of such an antibody retaining antigen binding capacities.

The antibody may have an effector function, such as ADCC or CDC, that is usually mediated by the Fc part (antibody constant region) of the antibody, or it may have no effector function, e.g. by lacking a Fc part or having a blocked, masked Fc part, in essence a Fc part that is not or insufficiently recognized by immune cells or immune system components, like the complement system.

The antibody or its fragment may be of any type, e.g. IgA, IgD, IgE, IgG, IgM. Preferred is IgG.

The terms “monoclonal antibody” or “monoclonal antibody composition” as used herein refer to a preparation of antibody molecules of a single amino acid composition or of a homogenous antibody population, i.e., a homogeneous population consisting of a whole immunoglobulin, or a fragment or derivative thereof. Such antibody may be selected from the group consisting of IgA, IgD, IgE, IgG, IgM, or a fragment thereof.

A “recombinant antibody” is an antibody which has been produced by a recombinantly engineered host cell. It is optionally isolated or purified.

A “human antibody” is one which possesses an amino acid sequence which corresponds to that of an antibody produced by a human cell or derived from a non-human source that utilizes human antibody repertoires or other human antibody-encoding sequences. This definition of a human antibody specifically excludes a humanized antibody comprising non-human antigen-binding residues.

The term “recombinant human antibody”, as used herein, is intended to include all human antibodies that are prepared, expressed, created or isolated by recombinant means, such as antibodies isolated from a host cell such as a NS0 or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. The recombinant human antibodies may have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

A “humanized” antibody refers to a chimeric antibody comprising amino acid residues from non-human hypervariable regions (HVRs) and amino acid residues from human framework regions (FRs). In certain embodiments, a humanized antibody will comprise substantially all of at least one, and typically two, variable domains, in which all or substantially all of the HVRs (e.g. complementary determining regions (CDRs)) correspond to those of a non-human antibody, and all or substantially the entire FRs correspond to those of a human antibody. A humanized antibody optionally may comprise at least a portion of an antibody constant region derived from a human antibody. A “humanized form” of an antibody, e.g. a non-human antibody, refers to an antibody that has undergone humanization.

“Binding” of a polypeptide (such as an immunoglobulin, an antibody, or generally an antigen-binding molecule or a fragment thereof) means “having affinity for” or “having specificity for” a certain epitope, antigen or protein (or for at least one part, fragment or epitope thereof). The terms “binding” and “specific binding” referring to the binding of the antibody or antigen binding moiety to an epitope of the antigen may be determined in an in vitro assay, preferably in a plasmon resonance assay (BIAcore®, GE Healthcare Uppsala, Sweden) with purified wild-type antigen.

Generally, the term “specificity” refers to the number of different types of antigens or epitopes to which a particular antigen-binding molecule (such as an antibody described herein) can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/KD). As will be clear to the skilled person, affinity can be determined in a manner known in the art, depending on the specific antigen of interest. Avidity is the measure of the strength of binding between an antigen-binding molecule (such as an immunoglobulin, an antibody, or generally an antigen-binding molecule or a fragment thereof) containing it and the pertinent antigen. Avidity is related to both the affinity between an epitope and its antigen binding site on the antigen-binding molecule and the number of pertinent binding sites present on the antigen-binding molecule.

An epitope is a region of an antigen that is bound by an antigen-binding molecule (such as an antibody described herein). The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three-dimensional structural characteristics, and/or specific charge characteristics. Conformational and non-conformational epitopes are distinguished in that the binding to the former but not the latter is lost in the presence of denaturing solvents.

The expressions “variable domain” or “variable region” or Fv as used herein denotes each of the pair of light and heavy chains which is involved directly in binding the antibody to the antigen. The variable domain of a light chain is abbreviated as “VL” and the variable domain of a heavy chain is abbreviated as “VH”. The variable light and heavy chain domains have the same general structure and each domain comprises four framework (FR) regions whose sequences are widely conserved, connected by three HVRs (or CDRs). The framework regions adopt a beta-sheet conformation and the CDRs may form loops connecting the beta-sheet structure. The CDRs in each chain are held in their three-dimensional structure by the framework regions and form together with the CDRs from the other chain the antigen binding site. The antibody's heavy and light chain CDR regions play a particularly important role in the binding specificity/affinity of the antibodies.

Within the context of this invention, reference to CDR's is based on the definition of Chothia (Chothia and Lesk, J. Mol. Biol. 1987, 196:901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

The term “constant domains” or “constant region” as used within the current application denotes the sum of the domains of an antibody other than the variable region. The constant region is not directly involved in binding of an antigen, but exhibits various effector functions. The “constant domains” as used in the antibodies disclosed herein are preferably from human origin, which is from a constant heavy chain region of a human antibody of the subclass IgG1, IgG2, IgG3, or IgG4 and/or a constant light chain kappa or lambda region. Such constant domains and regions are well known in the state of the art and e.g. described by Kabat et al. (“Sequence of proteins of immunological interest”, US Public Health Services, NIH Bethesda, MD, Publication No. 91).

The “Fc part” of an antibody is not involved directly in binding of an antibody to an antigen, but exhibits various effector functions. A “Fc part of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM, and several of these may be further divided into subclasses (isotypes), e.g. IgG1, IgG2, IgG3, and IgG4, IgA1, and IgA2. According to the heavy chain constant regions the different classes of immunoglobulins are called α,δ,ε,γ and μ, respectively. The Fc part of an antibody is directly involved in ADCC (antibody-dependent cell-mediated cytotoxicity) and CDC (complement-dependent cytotoxicity) based on complement activation, C1q binding and Fc receptor binding. Complement activation (CDC) is initiated by binding of complement factor C1q to the Fc part of most IgG antibody subclasses. While the influence of an antibody on the complement system is dependent on certain conditions, binding to C1q is caused by defined binding sites in the Fc part. Such binding sites are known in the state of the art and described e.g. by Boackle, R. J., et al, Nature 282 (1979) 742-743; Lukas, T. J., et al, J. Immunol. 127 (1981) 2555-2560; Brunhouse, R., and Cebra, J. J., Mol. Immunol. 16 (1979) 907-917; Burton, D. R., et al, Nature 288 (1980) 338-344; Thommesen, J. E., et al, Mol. Immunol. 37 (2000) 995-1004; Idusogie, E. E., et al, J. Immunol. 164 (2000) 4178-4184; Hezareh, M., et al, J. Virology 75 (2001) 12161-12168; Morgan, A., et al, Immunology 86 (1995) 319-324; EP 0 307 434. Such binding sites are e.g. L234, L235, D270, N297, E318, K320, K322, P331 and P329 (numbering according to EU index of Kabat, E. A., see below). Antibodies of subclass IgG1, IgG2 and IgG3 usually show complement activation and C1q and C3 binding, whereas IgG4 do not activate the complement system and do not bind C1q and C3. The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain.

The expression “anti-HER2 antibody” refers to an antibody that binds, in particular binds specifically, to the HER2 protein. Anti-HER2 antibodies inhibit HER2 activation or downstream signaling by various mechanisms. As non-limiting examples, anti-HER2 antibodies can prevent ligand binding, receptor activation or receptor signal propagation, result in reduced HER2 expression or localization to the cell surface, inhibit HER2 cleavage, or induce antibody-mediated cytotoxicity. In preferred embodiments, the expression “anti-HER2 antibody” is thus intended as a synonym and is replaceable with “inhibitory antibody binding specifically to HER2”. Anti-HER2 antibodies used for the treatment of cancer are typically monoclonal, although polyclonal antibodies are not excluded by the term. The anti-HER2 antibody can bind to one or more different epitopes on HER2. An anti-HER2 antibody can comprise one HER2 binding moiety or multiple HER2 binding moieties, such as two, three, four or more HER2 binding moieties. Preferably, the anti-HER2 antibody comprises one or two HER2 binding moieties. Any one or more anti-HER2 antibody(ies) can be used according to the invention. Preferably, “anti-HER2 antibody” as used herein is as defined in any of the aspects or embodiments of WO 2025/132522, which is herein incorporated by reference in its entirety.

In another aspect, the anti-HER2 antibody is an inhibitory antibody. The expression “inhibitory antibody” is intended as a synonym of and replaceable with “antagonistic antibody”. An “inhibitory antibody” or “antagonistic antibody” within the meaning of this invention is an antibody that inhibits the interaction of HER2 with its ligand(s) or receptor(s).

In another aspect, the anti-HER2 antibody binds specifically to HER2.

In another aspect, the anti-HER2 antibody binds specifically to an extracellular domain of HER2.

In another aspect, the anti-HER2 antibody is an inhibitory antibody binding specifically to HER2.

In another aspect, the anti-HER2 antibody is an inhibitory antibody binding specifically to an extracellular domain of HER2.

In another aspect, the combination partner is selected from the group consisting of trastuzumab, pertuzumab, margetuximab, zanidatamab, disitamab, anvatabart, hersintuzumab, anbenitamab and a combination thereof.

In another aspect, the combination partner is selected from the group consisting of trastuzumab, pertuzumab, margetuximab and a combination thereof.

As used herein in any embodiment, “trastuzumab”, “pertuzumab”, “margetuximab”, “zanidatamab”, “hersintuzumab”, “disitamab”, “anvatabart” and “anbenitamab” refer to the respective antibodies commonly known in the art with those names, in particular as further defined hereinbelow. The additional explanations hereinbelow are provided merely for the sake of clarity and are not intended to be limiting.

Trastuzumab, also known as Herceptin, is a humanized IgG1 monoclonal antibody that binds to HER2, in particular to its extracellular domain, especially to the HER2 domain that binds to another HER2 protein. The mechanism of action underlying the antitumour effect of trastuzumab has not yet been fully determined, and may in fact involve several different mechanisms. Trastuzumab may exert its effects by the activation of antibody-dependent cellular cytotoxicity, prevention of HER2 dimerization, the inhibition of the cleavage of the extracellular domain of HER2, interactions with signalling pathways, cell-cycle arrest during the G1 phase, induction of apoptosis or inhibition of angiogenesis. HER2 downregulation has also been suggested as a possible mechanism. Trustuzumab is disclosed in e.g., WO 92/22653, which is herein incorporated by reference.

Pertuzumab, also known as Perjeta, is a humanized IgG1 monoclonal antibody that binds to HER2, in particular to the extracellular domain of HER2, especially to the extracellular dimerization subdomain of HER2 receptor. Pertuzumab reduces HER2 intracellular signalling by preventing HER2 from forming heterodimers with other HER receptors, such as HER3. Pertuzumab is disclosed in e.g., U.S. Pat. Nos. 6,949,245 and 7,862,817 each of which is herein incorporated by reference.

Margetuximab, also known as Margenza, is a mouse/human chimeric IgG1 monoclonal antibody that binds to HER2, in particular to its extracellular domain, especially to the HER2 domain that binds to another HER2 protein. Margetuximab is disclosed in e.g., U.S. Pat. No. 8,802,093 which is herein incorporated by reference.

Zanidatamab, also known as ZW25 or Ziihera, is a humanised, bispecific/biparatopic, IgG1-like, monoclonal antibody that binds to HER2, in particular to the juxtamembrane extracellular domain and the dimerisation domain of HER2. Zanidatamab is disclosed in e.g., WO 2015/077891 and Weisser N. E., et al.; Nature Communications (2023) 14:1394, which are herein incorporated by reference.

Hersintuzumab is a humanized IgG1 monoclonal antibody that binds to HER2, in particular to the extracellular domain of HER2, especially to an epitope that is distinct from those recognised by trastuzumab and pertuzumab. Hersintuzumab is disclosed in e.g., U.S. Pat. No. 10,167,342 which is herein incorporated by reference.

Anbenitamab, also known as KN026, is a bispecific antibody that binds to HER2, in particular to the extracellular domain of HER2, especially to two distinct epitopes of the extracellular dimerization domain of HER2. It comprises heavy chains of trastuzumab and pertuzumab with a common light chain. Anbenitamab is disclosed in e.g. WO 2016/110267 which is herein incorporated by reference.

In another aspect, the combination partner is selected from the group consisting of trastuzumab, pertuzumab and a combination thereof.

In another aspect, the combination partner is trastuzumab or pertuzumab.

In another aspect, the combination partner is a combination of anti-HER2 antibodies, such as a combination of trastuzumab and pertuzumab.

In another aspect, the combination partner is a combination of trastuzumab and pertuzumab. Therefore, in an aspect, the invention provides zongertinib or a pharmaceutically acceptable salt thereof for use in the prevention and/or treatment of cancer in combination with trastuzumab and pertuzumab, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein.

In another aspect, the combination partner is trastuzumab. Therefore, in an aspect, the invention provides zongertinib or a pharmaceutically acceptable salt thereof for use in the prevention and/or treatment of cancer in combination with trastuzumab, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein. In another aspect, the combination partner is pertuzumab. Therefore, in an aspect, the invention provides zongertinib or a pharmaceutically acceptable salt thereof for use in the prevention and/or treatment of cancer in combination with pertuzumab, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein. In another aspect, the combination partner is an anti-HER2 antibody-drug conjugate, wherein said anti-HER2 antibody-drug conjugate may be as defined in any aspect hereinabove.

In another aspect, the combination partner is selected from the group consisting of trastuzumab deruxtecan, trastuzumab emtansine, trastuzumab rezetecan, trastuzumab duocarmazine, zanidatamab zovodotin, disitamab vedotin, anvatabart opadotin and a combination thereof.

In another aspect, the combination partner is selected from the group consisting of trastuzumab deruxtecan and trastuzumab emtansine.

In another aspect, the combination partner is trastuzumab deruxtecan. Therefore, in an aspect, the invention provides zongertinib or a pharmaceutically acceptable salt thereof for use in the prevention and/or treatment of cancer in combination with trastuzumab deruxtecan, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein.

In another aspect, the combination partner is trastuzumab emtansine. Therefore, in an aspect, the invention provides zongertinib or a pharmaceutically acceptable salt thereof for use in the prevention and/or treatment of cancer in combination with trastuzumab emtansine, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein.

In another aspect, the combination partner is:
a combination of anti-HER2 antibodies;
a combination of anti-HER2 antibody-drug conjugates;
a combination of at least one anti-HER2 antibody with at least one anti-HER2 antibody-drug conjugate; or
a combination of at least one anti-HER2 antibody with at least one anti-metabolite.

In another aspect, the combination partner is:
trastuzumab+pertuzumab;
trastuzumab deruxtecan+pertuzumab; or
trastuzumab+capecitabine.

Therefore, in an aspect, the invention provides zongertinib or a pharmaceutically acceptable salt thereof for use in the prevention and/or treatment of cancer in combination with trastuzumab deruxtecan and pertuzumab, wherein the cancer comprises at least one metastasis or lesion in the central nervous system. In this aspect, zongertinib, the cancer, the metastasis or lesion, the dosing schedule, the line of administration and/or the pharmaceutical composition can be as defined in any aspect or embodiment described herein.

Therefore, in an aspect, the invention provides zongertinib or a pharmaceutically acceptable salt thereof for use in the prevention and/or treatment of cancer in combination with trastuzumab and capecitabine, wherein the cancer comprises at least one metastasis in the central nervous system. In this aspect, zongertinib, the cancer, the metastasis, the dosing schedule, the line of administration and/or the pharmaceutical composition can be as defined in any aspect or embodiment described herein.

### Pharmaceutical Composition

Another aspect relates to a pharmaceutical composition comprising zongertinib or a pharmaceutically acceptable salt thereof and at least one pharmaceutically acceptable excipient for use in the prevention and/or treatment of cancer, wherein the cancer comprises at least one metastasis in the central nervous system.

Another aspect relates to a method of treating and/or preventing cancer in a patient in need thereof, wherein the method comprises administering to the patient a pharmaceutical composition comprising zongertinib or a pharmaceutically acceptable salt thereof and at least one pharmaceutically acceptable excipient, wherein the cancer comprises at least one metastasis in the central nervous system. In an embodiment, such method comprises administering to the patient a therapeutically effective amount of zongertinib or the pharmaceutically acceptable salt thereof.

Another aspect relates to a use of a pharmaceutical composition comprising zongertinib or a pharmaceutically acceptable salt thereof and at least one pharmaceutically acceptable excipient for the manufacture of a medicament for the treatment and/or prevention of cancer, wherein the cancer comprises at least one metastasis in the central nervous system.

Another aspect relates to a pharmaceutical composition comprising zongertinib or a pharmaceutically acceptable salt thereof and at least one pharmaceutically acceptable excipient for use in the prevention and/or treatment of metastatic cancer, wherein the metastatic cancer comprises at least one lesion in the central nervous system.

Another aspect relates to a method of treating and/or preventing metastatic cancer in a patient in need thereof, wherein the method comprises administering to the patient a pharmaceutical composition comprising zongertinib or a pharmaceutically acceptable salt thereof and at least one pharmaceutically acceptable excipient, wherein the metastatic cancer comprises at least one lesion in the central nervous system.

Another aspect relates to a use of a pharmaceutical composition comprising zongertinib or a pharmaceutically acceptable salt thereof and at least one pharmaceutically acceptable excipient for the manufacture of a medicament for the treatment and/or prevention of metastatic cancer, wherein the metastatic cancer comprises at least one lesion in the central nervous system.

Another aspect relates to a pharmaceutical composition comprising zongertinib or a pharmaceutically acceptable salt thereof and at least one pharmaceutically acceptable excipient for use in the prevention and/or treatment of metastatic cancer, wherein the metastatic cancer comprises or is metastatic cancer to the central nervous system.

Another aspect relates to a method of treating and/or preventing metastatic cancer in a patient in need thereof, wherein the method comprises administering to the patient a pharmaceutical composition comprising zongertinib or a pharmaceutically acceptable salt thereof and at least one pharmaceutically acceptable excipient, wherein the metastatic cancer comprises or is metastatic cancer to the central nervous system.

Another aspect relates to a use of a pharmaceutical composition comprising zongertinib or a pharmaceutically acceptable salt thereof and at least one pharmaceutically acceptable excipient for the manufacture of a medicament for the treatment and/or prevention of metastatic cancer, wherein the metastatic cancer comprises or is metastatic cancer to the central nervous system. Preferably, in these aspects, zongertinib or the pharmaceutically acceptable salt thereof is administered according to the doses and dose regimes described hereinabove. Also preferably, in these embodiments, the (metastatic) cancer and or the metastasis or lesion is as defined herein (in any aspect or embodiment). Additionally or alternatively, in these aspects, zongertinib may be administered as first, second or further line and/or as monotherapy and/or in combination with an additional intervention such as a combination partner as described herein.

All aspects and embodiments described above relative to zongertinib or the pharmaceutically acceptable salt thereof for use in the treatment of (metastatic) cancer are applicable to the pharmaceutical composition for use in the treatment of (metastatic) cancer. The same applies to aspects and embodiments relating to methods of prevention/treatment or to uses.

The term “pharmaceutically acceptable excipient” refers to a non-toxic component that does not destroy the pharmacological activity of the compound with which it is formulated. The pharmaceutical composition may contain conventional non-toxic pharmaceutically acceptable excipients. Pharmaceutically acceptable excipients that may be used in the compositions of this invention include fillers, disintegrants, glidants, lubricants, and coating agents. The compositions may comprise further pharmaceutically acceptable excipients selected from buffers, dispersion agents, surfactants, wetting agents, emulsifiers, suspending agents, preservatives, antioxidants, opaquing agents, processing aids, colorants, sweeteners, perfuming agents, flavoring agents, diluents and other known additives usable in the manufacturing of a pharmaceutical product.

In a preferred embodiment, zongertinib or the pharmaceutical composition is administered orally. Zongertinib or the pharmaceutical composition may be administered as a tablet, hard or soft gelatin capsule, pill, granules or a suspension. In preferred embodiments, zongertinib or the pharmaceutical composition is in the form of a tablet.

Preferably, the pharmaceutical composition as defined herein comprises the solid dispersion as defined in WO 2024/133289, which is herein incorporated by reference.

Preferably, the pharmaceutical composition as defined herein corresponds to the pharmaceutical composition as defined in WO 2024/133289, which is herein incorporated by reference.

In one embodiment, the pharmaceutical composition comprises, consists essentially of or consists of: approximately 60 mg zongertinib, approximately 60 mg hypromellose acetate succinate, approximately 80 mg microcrystalline cellulose, approximately 168 mg mannitol, approximately 20 mg croscarmellose sodium, approximately 6 mg colloidal silicone dioxide and approximately 6 mg sodium stearyl fumarate. In an embodiment, zongertinib pharmaceutical compositions comprise, consist of, or consist essentially of 60 mg of zongertinib and the following inactive ingredients: colloidal silicon dioxide, croscarmellose sodium, hypromellose acetate succinate, mannitol, microcrystalline cellulose, and sodium stearyl fumarate. In addition, the film-coating contains the following inactive ingredients: ferric oxide (yellow), glycerol mono and dicaprylocaprate, polyvinyl alcohol, sodium lauryl sulfate, talc, and titanium dioxide.

In an embodiment, the pharmaceutical composition is characterized by having an x-ray powder diffractogram (XRPD) comprising no diffraction peak at 2-theta angles equal or below 10.0°, when measured at a temperature in the range of from 20 to 30° C. and with Cu-Kα radiation having a wavelength of 1.54056 Å or 1.54184 Å.

Unless otherwise defined, the technical and scientific terms used herein have the same meaning as commonly understood by one of ordinary skill in the art to which this invention belongs.

Features and advantages of the present invention will become apparent from the following detailed examples, which illustrate the principles of the invention by way of example without restricting its scope.

### Example 1—Protocol of an Open Label, Phase I Dose Escalation Trial, with Dose Confirmation and Expansion, of Zongertinib as Monotherapy in Patients with Advanced or Metastatic Solid Tumors with HER2 Aberrations

This is a First-in-Human dose escalation and expansion trial to determine the Maximum Tolerated Dose (MTD) and explore safety, pharmacokinetics, pharmacodynamics and first signs of efficacy of zongertinib as monotherapy in patients with HER2 aberration-positive advanced or metastatic solid tumors.

The trial has two parts: Phase Ia, which is the dose escalation part, and Phase Ib which is the dose expansion part. Phase Ia includes consecutive cohorts of patients treated with escalating doses of zongertinib. Phase Ib is split into different cohorts, including:
Cohort 1: pre-treated non-small cell lung cancer (NSCLC) with a HER2 tyrosine kinase domain (TKD) mutation;
Cohort 2: treatment-naïve NSCLC with a HER2 TKD mutation;
Cohort 3: NSCLC with a non-TKD HER2 mutation or HER2 TKD mutation-positive squamous NSCLC, previously treated;
Cohort 4: NSCLC with active brain metastases with a HER2 TKD mutation;
Cohort 5: NSCLC with a HER2 TKD mutation and previous treatment with a HER2 directed antibody-drug conjugate (ADC).

Patients with stable and/or asymptomatic brain metastases are eligible for Phase Ia and cohorts 1, 2, 3 and 5 of Phase Ib. Patients with active brain metastases are eligible for cohort 4 of Phase Ib.

### 1.1 Objectives:

The main objectives of the dose escalation part of the trial are to:
Investigate the safety, tolerability, and pharmacokinetics (PK) of escalating doses of zongertinib as monotherapy administered orally bis in die (BID, twice daily dosing) or quaque die (QD, once a day) in patients with advanced and/or metastatic solid tumors harbouring HER2 aberrations;
Determine the MTD and/or the Recommended Phase II Dose (RP2D, corresponding to the dose recommended for dose expansion in Phase Ib) of zongertinib monotherapy administered orally in any studied regimen.

The primary objective of Phase Ib is the assessment of the anti-tumor activity of zongertinib, where the primary measure of interest is the objective tumor response rate in the different cohorts.

Secondary objectives are to assess further measures of efficacy, e.g. via DoR, DC, PFS, OR and DC, the last two via RANO-BM for patients with CNS lesions at baseline. Additional secondary objectives are to evaluate safety (by determining the number of patients experiencing DLTs during the entire treatment period) and the assessment of patient-reported outcomes.

### 1.2 Endpoints:

The primary endpoints of the dose escalation part of the trial are:
MTD, defined as the highest dose with less than 25% risk of the true Dose Limiting Toxicity (DLT) rate being equal to or above 33% during the MTD evaluation period in any studied regimen;
Number of patients with DLTs in the MTD evaluation period.

The MTD evaluation period is defined as the first 21 days of treatment (first cycle).

The primary endpoints of the dose expansion part of the trial are:
For cohort 1, 2, and 5: objective response (OR) defined as best overall response of complete response (CR) or partial response (PR), where best overall response is determined according to Response Evaluation Criteria In Solid Tumors (RECIST) version 1.1, from the first treatment administration until the earliest of disease progression, death or last evaluable tumor assessment before start of subsequent anti-cancer therapy, loss to follow-up or withdrawal of consent;
For cohort 3: OR according to RECIST version 1.1 by investigator assessment;
For cohort 4: OR in lesions in the central nervous system (CNS) assessed by central independent review, according to Response Assessment in Neuro-Oncology criteria for Brain Metastases (RANO-BM).

The secondary endpoints of the dose escalation part of the trial are:
Number of patients experiencing DLTs during the entire treatment period;
PK parameters of zongertinib after first and multiple dose administration of the compound on Day 1 and 15 (if feasible):
   Cₘₐₓ: maximum measured concentration of zongertinib in plasma;
   AUC₀₋ₜ₂: area under the concentration-time curve of zongertinib in plasma.

The secondary endpoints of the dose expansion part of the trial are:

### For Cohort 1, 2, and 5:

DoR according to RECIST 1.1, defined as the time from first documented complete response (CR) or partial response (PR) until the earliest of disease progression or death among patients with objective response as assessed by central independent review;
DC, defined as best overall response of complete response (CR) or partial response (PR) or stable disease (SD), where best overall response is defined according to RECIST version 1.1 as assessed by central independent review, from first treatment administration until the earliest of disease progression, death or last evaluable tumor assessment before start of subsequent anti-cancer therapy, loss to follow-up or withdrawal of consent;
PFS, defined as the time from first treatment administration until tumor progression according to RECIST version 1.1 as assessed by central independent review, or death from any cause, whichever occurs earlier;
OR and DC according to RANO-BM criteria for patients with CNS lesions at baseline, as assessed by central independent review;
OR and DC in CNS lesions according to RECIST 1.1 criteria as assessed by central independent review for patients with CNS lesions at baseline.

For Cohort 3 only:
DoR, DC and PFS according to RECIST 1.1 as assessed by investigator;
OR and DC according to RANO-BM criteria for patients with CNS lesions at baseline, as assessed by the investigator.

For Cohort 4 only:
DoR, DC and PFS according to RANO-BM by central independent review;
DoR, DC and PFS in CNS lesions according to RECIST 1.1 by central independent review;
OR, DoR, DC and PFS according to RECIST 1.1 by central independent review.

For all cohorts:
Overall survival (OS), defined as time from first treatment administration until death from any cause;
Number of patients experiencing DLTs during the entire treatment period;
Change from baseline to C5D1 (cycle 5, day 1) in EORTC QLQ-C30 physical functioning domain score;
Change from baseline to C5D1 in NSCLC-SAQ total score;
Change from baseline to C5D1 in EORTC IL46 item score.

EORTC QLQ-C30, NSCLC-SAQ and EORTC IL46 are patient reported outcome measures (PROMs). Specifically, EORTC QLQ-C30 (IL19) physical functioning domain score is a 5-item functional scale from the EORTC QLQ-C30, which is a questionnaire specific for the assessment of health-related quality of life (QoL) in patients with cancer. NSCLC-SAQ is a 7-item PROM for use in adults to assess symptoms of advanced NSCLC. It contains five domains and accompanying items that were identified as symptoms of NSCLC: cough (1 item), pain (2 items), dyspnea (1 item), fatigue (2 items), and appetite (1 item). EORTC IL46 (item 168) is a validated single-item question that assesses overall side effect impact. Other PROM may be measured in the course of the study.

If assessable and applicable, the following further endpoints are evaluated in the dose escalation part:
Number of patients experiencing adverse events (AEs) during the on-treatment period;
OR, DC and DoR;
Duration of disease control (DoDC), defined as the time from first treatment administration until the earliest of disease progression or death, among patients with disease control;
Tumor shrinkage according to RECIST 1.1, defined as the best (smallest) percentage change from baseline of the sum of longest diameter of target lesions assessed by the investigator across all on-treatment tumor assessments;
If data allows, the PK parameters to be calculated for zongertinib as monotherapy include:
   AUC_{0-∞}: area under the concentration-time curve in plasma over the time interval from 0 extrapolated to infinity;
   AUC_{0-tz}: area under the plasma concentration-time curve over the time interval from 0 to the last measured time point (t_{z});
   Cₘᵢₙ: minimum measured plasma concentration in plasma;
   t_{1/2}: terminal half-life of the analyte in plasma;
   tₘₐₓ: time from dosing to Cₘₐₓ in plasma.

If data allows, the following further endpoints are evaluated in the dose expansion part:
PK parameters,
Pharmacodynamic parameters of zongertinib;
Duration of disease control (DoDC) according to RECIST 1.1 as assessed by the investigator (for Cohort 3 only);
NSCLC-SAQ domain scores (fatigue, cough, dyspnoea, pain, and appetite), EORTC IL46, and PRO-CTCAE items (mouth/throat sores, taste changes, nausea, vomiting, diarrhoea, rash, skin dryness, itching, numbness & tingling) collected from baseline until C9D1 (cycle 9, day 1);
For Cohorts 1, 2, 4, and 5 only: DoDC according to RECIST 1.1 as assessed by central independent review;
For Cohorts 1, 2, 4, and 5: OR, DoR, DC, DoDC, tumor shrinkage, and PFS of non-CNS areas according to RECIST 1.1 as assessed by central independent review;
For Cohort 3: OR, DoR, DC, DoDC, tumor shrinkage, and PFS of non-CNS areas according to RECIST 1.1 as assessed by the investigator;
Cohorts 1, 2, and 5: DoR and DoDC according to RANO-BM as assessed by central independent review;
For Cohorts 1, 2, and 5: DoR in CNS lesions and DoDC in CNS lesions according to RECIST 1.1 as assessed by central independent review;
Cohort 3: DoR and DoDC according to RANO-BM as assessed by the investigator;
Cohort 4: DoDC according to RANO-BM by central independent review;
Cohort 4: DoDC in CNS lesions according to RECIST 1.1 by central independent review.

For clarity, in Phase Ia and Phase Ib, tumor response (RECIST v1.1) was assessed in patients with and without brain metastases. In addition, in Phase Ib, CNS response (RANO-BM; blinded independent central review, BICR) was included as a secondary endpoint (primary endpoint in Cohort 4). With a protocol amendment, CNS response (RECIST 1.1; BICR) was added as a secondary endpoint in Cohorts 1, 2 and 5 (and partially in Cohort 4). For the RECIST evaluation, brain metastases were not selected as the target lesion; while for the RANO-BM evaluation, all eligible patients had target lesions in the brain.

Further endpoints may be defined.

### 1.3 Doses:

The starting doses of the dose escalation part of the trial are:
BID Schedule: 15 mg twice daily (i.e. total daily dose of 30 mg)
QD Schedule: 60 mg once daily (if not proposed otherwise by the Dose Escalation Committee, DEC).

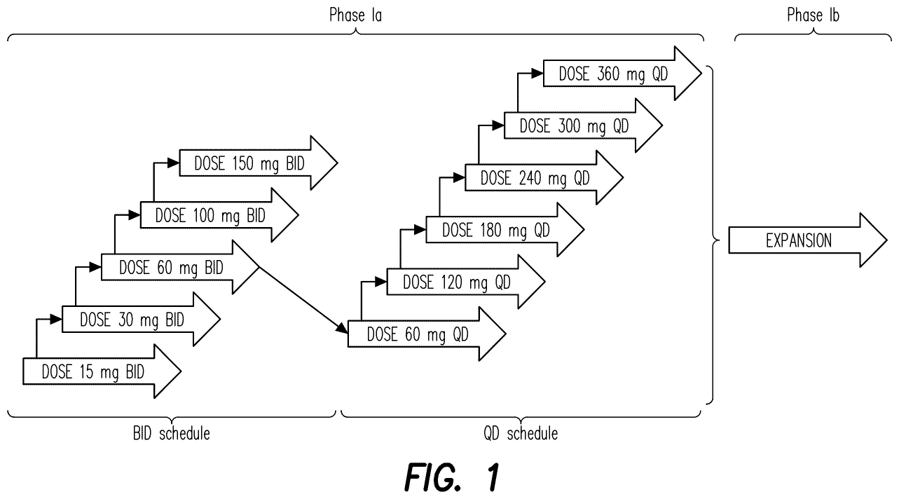

Successive cohorts of patients receive increasing doses of zongertinib until the MTD is reached. Dose-escalation steps are determined by the DEC, guided by a Bayesian logistic regression model (BLRM) with overdose control (EWOC). Dose escalation levels are shown in FIG. 1. In the dose expansion part of the trial, patients are randomized to receive either 120 mg or 240 mg QD. One of the two doses is selected at interim analysis. In cohort 1, patients were initially randomly assigned to receive zongertinib at a dose of 120 mg or 240 mg once daily. Patients in cohorts 5 and 3 initially received 240 mg daily. After an interim analysis of data from cohort 1, subsequently recruited patients across all cohorts received zongertinib at a dose of 120 mg. In both parts, zongertinib is administered in reiterated treatment cycles of 3 weeks as long as the patient has clinical benefit or until undue drug toxicity or withdrawal of consent, whichever occurs first.

### 1.4 Patients

The dose escalation part enrolls patients with advanced, unresectable and/or metastatic solid tumors with an aberration of the HER2 gene.

The dose expansion part enrolls patients with advanced or metastatic refractory NSCLC harbouring mutations in the HER2 gene.

Inclusion and exclusion criteria are provided below.

Main inclusion criteria for all parts (Phase Ia and Ib):
Histologically or cytologically confirmed diagnosis of an advanced, unresectable and/or metastatic non-haematologic malignancy. Patient must show presence of at least one measurable non-CNS lesion according to RECIST 1.1 and for Phase Ib cohort 4, additionally, presence of at least one measurable brain lesion according to RANO-BM. Note: Patients with asymptomatic (i.e. no clinical (neurological) symptoms) brain lesions are eligible for Phase Ia and cohorts 1, 2, 3, and 5 of Phase Ib and patients with active brain metastases are eligible for cohort 4 of Phase Ib;
Eastern Cooperative Oncology Group (ECOG) performance-status score (PS) of 0 or 1;
Availability and patient willingness to provide a sample of tumor for confirmation of the patient's HER2 status. This sample can be archival material obtained at any time prior to study enrollment;
Patients willing and able to comply with the blood sampling and tumor biopsy requirements for PK, pharmacodynamics and biomarker analyses;
Adequate organ function as routinely measured in the field;
Recovered from any previous therapy-related toxicity to ≤Common Terminology Criteria for Adverse Events (CTCAE) Grade 1 at start of treatment (except for alopecia, stable sensory neuropathy and hypothyroidism (patients on thyroid replacement therapy) which must be ≤CTCAE Grade 2).
Life expectancy of at least 12 weeks at the start of treatment in the opinion of the investigator.
At least 18 years of age at the time of consent or over the legal age of consent in countries where that is greater than 18 years.
Signed and dated written informed consent in accordance with International Council on Harmonisation-Good Clinical Practice (ICH-GCP) and local legislation prior to admission to the trial.
Male or female patients. Women of childbearing potential (WOCBP) and men who are able to father a child must be ready and able to use highly effective methods of birth control per ICH M3 (R2) that result in a low failure rate of less than 1% per year when used consistently and correctly.

Additionally, for Phase Ia:
Patients with a documented aberration of the HER2 gene comprising: EITHER overexpression by immunohistochemistry (IHC), gene copy-number increase by in-situ hybridization (ISH), non-synonymous gene mutation OR gene rearrangement/fusion of the HER2 or NRG-1 genes.
Patient who has failed conventional treatment or for whom no therapy of proven efficacy exists or who is not eligible for established treatment options. Patient must have exhausted, or not be a suitable candidate for, available treatment options known to prolong survival for their disease.

Additionally, for Phase Ib:
For cohort 1: Patients with documented HER2 TKD mutation positive non-squamous NSCLC who had received, in the advanced/metastatic setting, at least one line of systemic therapy that includes a platinum-based combination chemotherapy;
For cohort 2: Patients with documented HER2 TKD mutation positive non-squamous NSCLC who have not received any previous lines of therapy (neo or adjuvant chemo, chemoradio or radiotherapy is permitted if at least 6 months have elapsed prior to disease progression), i.e. patients that are treatment naïve for non-squamous NSCLC;
For cohort 3: Patients with documented HER2 non-TKD mutation positive NSCLC, and HER2 TKD mutation positive squamous NSCLC, who had received in the advanced/metastatic setting at least one line of systemic therapy that includes a platinum-based combination chemotherapy;
For cohort 4: Patients with documented HER2 TKD mutation positive NSCLC with active brain metastases who are either treatment naïve or who had received any line of previous treatment for NSCLC in the advanced/metastatic setting;
Patients in these cohorts 1 to 4 must have not received prior therapy with HER2 directed antibody-drug conjugates (ADC);
For cohort 5: Patients with documented HER2 TKD mutation positive non-squamous NSCLC who had received prior therapy with HER2 directed ADC in the advanced/metastatic setting and developed disease progression during or after completing this therapy.

Main exclusion criteria:
Major surgery (major according to the investigator's assessment) performed within 4 weeks prior to first trial treatment or planned within 6 months after screening;
Previous or concomitant malignancies other than the one treated in this trial within the last 2 years, except;
   effectively treated non-melanoma skin cancers,
   effectively treated carcinoma in situ of the cervix,
   effectively treated ductal carcinoma in situ,
   other effectively treated malignancy that is considered cured by local treatment;
Treatment with a systemic anti-cancer therapy or investigational drug within 21 days or 5 half-lives (whichever is shorter) of the first treatment with zongertinib;
Patients who must or wish to continue the intake of restricted medication or any drug considered likely to interfere with the safe conduct of the trial;
Previous treatment with zongertinib. For Phase Ib only: Previous treatment with any HER2 targeted treatment;
Radiotherapy within 2 weeks prior to first study treatment, except palliative radiotherapy to regions other than the chest, which is allowed up to 1 week prior to first study treatment. After the start of study treatment, CNS directed radiotherapies are allowed if a patient experiences isolated progression in CNS. The irradiated area cannot be used for further tumour response assessment.

Patients are also excluded if they have current or a history of uncontrolled or symptomatic brain and subdural metastases, unless considered stable by the investigator and local therapy is completed, or they have known leptomeningeal disease. Inclusion of patients with newly identified brain metastasis/es at screening is allowed if patients are asymptomatic, considered stable by the investigator and immediate central nervous system treatment is unlikely to be required.

As the skilled person will recognise, further inclusion and exclusion criteria may apply.

### 1.5 Compound:

Zongertinib is administered as film-coated tablets. A first formulation was developed in three dosage strengths: 5 mg (about 10 mm round), 20 mg (about 10 mm round) and 100 mg (oval, about 16×7 mm). This first formulation is administered in Phase Ia.

A spray dried dispersion formulation of zongertinib is used in Phase Ib. This formulation was developed in two dosage strengths: 15 mg (about 6 mm round) and 60 mg (about 7.2×14.6 mm oval), as described in Examples 6.2-B and 6.2-C in Table 17 of WO 2024/133289.

In addition to the drug substance, the tablets contain standard pharmaceutical excipients in common amounts.

### 1.6 Assessments:

Tumor assessments, including computed tomography and/or positron emission tomography scans of the chest, abdomen or pelvis, were performed by the investigator at screening (≤28 days prior to initiation of treatment), every 2 cycles (6 weeks+5 days), at the end of treatment visit (if not performed within the previous 3 weeks), and at the discretion of the investigator. Brain magnetic resonance imaging was performed in all eligible patients at screening, and (if clinically indicated) at all further assessments. Patients who withdrew from the study were categorized as non-responders.

Safety assessments were performed in all treated patients using physical examinations, assessments of vital signs, safety laboratory parameters, electrocardiograms, left ventricular ejection function, and by monitoring the occurrence of adverse events. Adverse events were assessed according to the National Cancer Institute CTCAE version 5.0.

Tumour response is evaluated according to RECIST Version 1.1 (Eisenhauer E A, Therasse P, Bogaerts J, Schwartz L H, Sargent D, Ford R, et al. New response evaluation criteria in solid tumours: revised RECIST guideline (version 1.1). Eur J Cancer. 2009; 45:228-247). RECIST 1.1 is used for a) whole-body assessment (classical RECIST 1.1) and b) the tumour assessment of non-CNS areas. In Phase Ia, RECIST Version 1.1 is only used for non-brain tumor assessments, i.e. of the non-CNS area. No whole-body RECIST assessment is performed in Phase Ia.

Tumor response is also evaluated according to Response Assessment in Neuro-Oncology (RANO criteria) for brain metastases (RANO-BM) (Chukwueke Ugonma N, Wen Patrick Y. Use of the Response Assessment in Neuro-Oncology (RANO) criteria in clinical trials and clinical practice. CNS Oncology 2019, 8 (1), CNS28). RANO-BM criteria are summarized in Table 0.

TABLE 0
Criteria for response assessment incorporating MRI and clinical
factors for brain metastases (RANO-BM) (Chukwueke Ugonma et al.)

| Criterion | CR | PR | SD | PD |
|---|---|---|---|---|
| Target lesions | None | ≥30% decrease | <30% decrease | ≥20% increase |
| | | in sum of lesion | relative to | in sum of lesion |
| | | diameter | baseline, but <20% | diameter |
| | | relative to | increase in | relative to nadir¹ |
| | | baseline | sum of lesion | |
| | | | diameter | |
| | | | relative to nadir | |
| Non-target | None | Stable or | Stable or | Unequivocal |
| lesions | | improved | improved | PD¹ |
| New lesion(s)² | None | None | None | Present¹ |
| Corticosteroids | None | Stable or | Stable or | NA³ |
| | | decreased | decreased | |
| Clinical status | Stable or | Stable or | Stable or | Worse¹ |
| | improved | improved | improved | |
| Requirement for | All | All | All | Any³ |
| response | | | | |

¹Progression occurs when this criterion is met.
²A new lesion is one that is not present on prior scans and is visible in a minimum of two projections. If a new lesion is equivocal, for example because of its small size, continued therapy can be considered, and follow-up assessment clarify if the new lesion is new disease. If repeat scans confirm there is definitely a new lesion, progression should be declared using the date of the initial scan showing the new lesion. For immunotherapy-based approaches, new lesions alone to do not define progression.
³Increase in corticosteroids alone is not taken into account in determining progression in the absence of persistent clinical deterioration

In addition, tumor response is evaluated centrally and independently (in a blinded fashion) according to CNS RECIST 1.1.

If there are multiple contrast-enhancing brain lesions, a maximum of five target lesions should be measured.

If a Phase Ib patient experiences isolated progression in CNS according to RANO-BM but has not progressed according to whole-body RECIST 1.1, the patient may have the CNS lesions treated with CNS directed therapies, if agreed with the sponsor, and remain on study treatment until PD according to RECIST 1.1 or any other discontinuation criteria are met.

For Cohort 1, 2, 4, and 5 in Phase Ib, all imaging related endpoints is also centrally and independently assessed (in a blinded fashion) according to RECIST 1.1 and RANO-BM if applicable.

### 1.7 Statistical Methods:

Dose escalation is guided by BLRM with EWOC that is fitted to binary toxicity outcomes (DLTs). The estimate of parameters is updated as data are accumulated using the BLRM. At the end of the dose escalation phase, the toxicity probability at each dose level are calculated to determine an estimate of the MTD.

For Cohort 1 of Phase Ib, the analysis of the primary endpoint OR by central independent review is performed with a one-sided z-test at the one-sided alpha level of 0.0125 (to account for the two doses investigated).

For Cohorts 2 and 5 of Phase Ib, the analysis of the primary endpoint OR by central independent review is performed with a one-sided z-test at the one-sided alpha level of 0.025.

For Cohort 3, no confirmatory testing is done. Only descriptive analyses are performed, in terms of the observed ORR and 95% CIs by the Wilson method. In addition, the primary endpoint OR by investigator assessment is analysed using a Bayesian Hierarchical Model (BHM) for the ORR, hereby leveraging available co-data from Cohorts 1, 2 and 3 in a meta-analytic Bayesian approach.

For Cohort 4 of Phase Ib, no confirmatory testing is done. Only descriptive analyses of the primary endpoint OR in the CNS by central review according to RANO-BM is conducted.

### Example 2—Results of the Dose Escalation Part (Phase Ia)

This example reports the results of the dose escalation part (Phase Ia) of Example 1. In Phase Ia, a quarter of patients had asymptomatic brain metastases (26% out of the total 105 patient with solid tumors and HER2 aberrations). These patients were heavily pretreated (>2 lines of previous therapy: 67%). The baseline characteristics of enrolled patients are reported in Table 1.

TABLE 1
Baseline characteristics of patients enrolled in Phase Ia.

| | All patients | | Brain metastases | |
|---|---|---|---|---|
| Characteristic | (n = 105) | | (n = 27) | |
| Median age, years (range) | 60 | (31-81) | 60 | (31-81) |
| Female, n (%) | 56 | (53.3) | 13 | (48.1) |

Country of recruitment, n (%)

| | | | | |
|---|---|---|---|---|
| USA | 31 | (29.5) | 12 | (44.4) |
| Netherlands | 23 | (21.9) | 0 | (0.0) |
| Japan | 44 | (41.9) | 10 | (37.0) |
| China | 7 | (6.7) | 5 | (18.5) |

Previous lines of therapy, n (%)

| | | | | |
|---|---|---|---|---|
| ≤2 | 38 | (36.2) | 9 | (33.3) |
| >2 | 67 | (63.8) | 18 | (66.7) |

Tumor type, n (%)

| | | | | |
|---|---|---|---|---|
| NSCLC | 54 | (51.4) | 25 | (92.6) |
| Colorectal cancer | 14 | (13.3) | 0 | (0.0) |
| Breast cancer | 12 | (11.4) | 2 | (7.4) |
| Esophageal cancer | 5 | (4.8) | 0 | (0.0) |
| Other tumors* | 20 | (19.0) | 0 | (0.0) |
| HER2m+** | 57 | (54.3) | 21 | (77.8) |

*cervical (n = 3), endometrial (n = 3), biliary tree (n = 2), gastrointestinal tract (n = 3), genitourinary system, head and neck, mediastinum, ovary, pancreas, small intestine, uterine, vulva, and other (all n = 1)
**HER2 mutation-positive. Patients without HER2m+ tumors had other HER2 aberrations, as defined in the protocol of Example 1.

Zongertinib demonstrated manageable safety and promising efficacy in patients with HER2 aberration-positive solid tumors in Phase Ia, including in those with brain metastases at baseline. Safety findings from Phase Ia are reported in Table 2 and 3.

TABLE 2
Treatment-related adverse events (TRAEs) in Phase Ia

| | All patients (N = 105) | | Brain metastases (n = 27) | |
|---|---|---|---|---|
| TRAEs (%) | Any | Grade ≥3 | Any | Grade ≥3 |
| Any TRAE* | 81.9 | 10.5 | 77.8 | 25.9 |
| Diarrhea | 50.5 | 1.0 | 44.4 | 0.0 |
| Rash^{†} | 15.2 | 2.0 | 14.8 | 7.4 |
| Anemia | 10.5 | 0.0 | 11.1 | 0.0 |
| ALT increased | 9.5 | 3.8 | 25.9 | 14.8 |
| Decreased appetite | 9.5 | 1.0 | 11.1 | 3.7 |
| AST increased | 8.6 | 1.9 | 18.5 | 7.4 |
| Fatigue | 8.6 | 0 | 7.4 | 0.0 |
| Nausea | 8.6 | 1.0 | 7.4 | 3.7 |
| Stomatitis | 8.6 | 0.0 | 11.1 | 0.0 |

*TRAEs that occurred in ≥9 out of all patients are shown
^{†}Combined term, includes rash and rash maculo-papular

There was only one grade 4 TRAE (thrombocytopenia, 240 mg QD) and no grade 5 TRAE. 3 patients had serious TRAEs, 2 of which had brain metastasis.

TABLE 3
Dose limiting toxicities (DLTs) in Phase Ia. DLTs
in bold are for patients with brain metastases.

| Dose | n = 8 | Dose-limiting toxicities (DLTs) |
|---|---|---|
| 60 mg BID | 1 | Grade 2 edema |
| 150 mg BID | 1 | Grade 2 diarrhea |
| 180 mg QD | 1 | Grade 3 ALT increased |
| | | Grade 2 ALT increased |
| | | Grade 2 blood bilirubin increased |
| 240 mg QD | 2 | Grade 3 diarrhea (MTD period) |
| | | Grade 4 thrombocytopenia |
| 300 mg QD | 1 | Grade 2 ejection fraction decreased |
| 360 mg QD | 2 | Grade 3 ALT increased |
| | | Grade 3 AST increased |
| | | Grade 2 acute interstitial pneumonitis |
| | | Grade 3 platelet count decreased (MTD period) |

8 patients had DLTs, 2 of which during the MTD evaluation period. MTD was not reached with either BID or QD schedule.

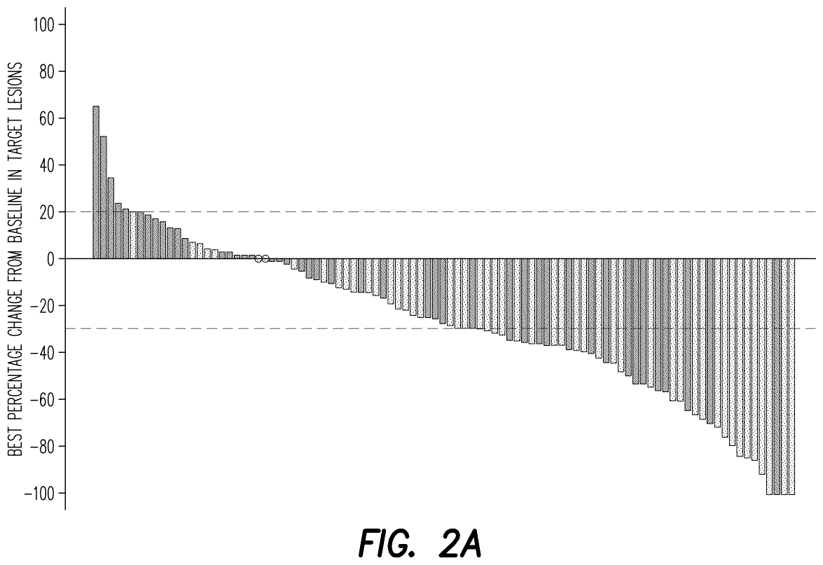

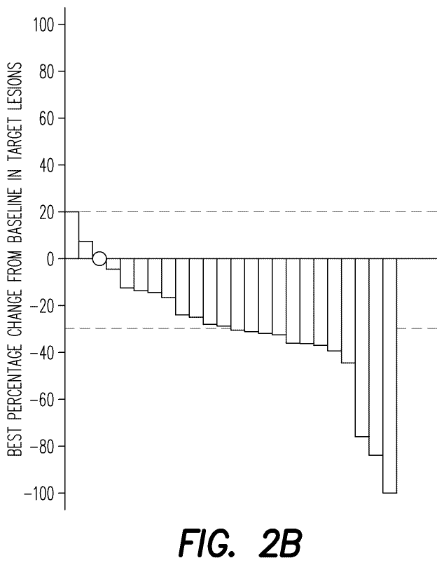

Efficacy Results from Phase Ia are Reported in FIG. 2.

Response rates were similar among all patients and those with brain metastases. Of the 99 evaluable patients in Phase Ia, the overall response rate (ORR) by RECIST v1.1 was 32.3% and the disease control rate (DCR) was 87.9%. 53 of these patients had NSCLC. For them, the ORR was 35.8% and the DCR 94.3%. The median DoR was 12.7 months with a (95% confidence interval, CI, 6.9—not calculated, NC). The median PFS were 8.0 months (BID, 95% CI: 2.8-NC) and 8.3 months (QD, 95% CI: 5.5-13.8).

Of the 24 patients with brain metastases evaluable by RECIST v1.1, the ORR and DCR in non-CNS lesions were 25% and 95.8%, respectively. Of the 26 patients with brain metastases evaluable by RANO-BM, the ORR and DCR in CNS lesions were 7.7% and 73.1%, respectively. Of the 23 patients with NSCLC with brain metastases evaluable by RANO-BM, the ORR and DCR in CNS lesions were 8.7% and 78.3%, respectively.

Confirmed response rate by RECIST v1.1 across all doses was similar to the overall population (25% and 32%, respectively).

Preliminary evidence of intracranial activity was observed, with up to −49% tumor shrinkage in brain lesion size as per investigator assessment according to RANO-BM.

### Example 3—Results from Cohort 1 of the Dose Expansion Part (Phase Ib)

This example reports the results from cohort 1 of the dose expansion part (Phase Ib) of Example 1. In cohort 1, patients with pre-treated NSCLC with a HER2 TKD mutation (TKDm+) were treated with 120 mg QD or 240 mg QD zongertinib. The baseline characteristics of enrolled patients are reported in Tables 4a (patients divided by dose) and 4b (patients with brain metastases, regardless of dose). At the cut-off date, 132 patients with pretreated HER2-mutant NSCLC had received zongertinib in cohort 1 of Phase Ib. Of these, 41% had asymptomatic brain metastases. Patient characteristics were similar between patients with brain metastases and the total population.

TABLE 4a
Baseline characteristics of patients enrolled
in cohort 1 of Phase Ib, divided by dose.

| | 120 mg | 240 mg | Total |
|---|---|---|---|
| | (n = 75) | (n = 57) | (N = 132) |
| Median age, years | 62 (30-80) | 62 (36-82) | 62 (30-82) |
| (range) | | | |

Sex, n (%)

| | | | |
|---|---|---|---|
| Female | 51 (68) | 25 (44) | 76 (58) |
| Male | 24 (32) | 32 (56) | 56 (42) |

Race, n (%)

| | | | |
|---|---|---|---|
| Asian | 40 (53) | 33 (58) | 73 (55) |
| White | 24 (32) | 15 (26) | 39 (30) |
| Missing | 11 (15) | 9 (16) | 20 (15) |

ECOG PS, n (%)

| | | | |
|---|---|---|---|
| 0 | 28 (37) | 17 (30) | 45 (34) |
| 1 | 47 (63) | 40 (70) | 87 (66) |

Number of previous lines of systemic anticancer treatment, n (%)

| | | | |
|---|---|---|---|
| 1 | 42 (56) | 28 (49) | 70 (53) |
| 2 | 12 (16) | 16 (28) | 28 (21) |
| ≥3 | 21 (28) | 13 (23) | 34 (26) |
| Brain metastases at | 28 (37) | 26 (46) | 54 (41) |
| baseline | | | |

TABLE 4b
Baseline characteristics of patients enrolled in cohort
1 of Phase Ib, focused on patients with brain metastases.

| | Total | Brain metastases |
|---|---|---|
| | N = 132 | n = 54 |
| Median age, years (range) | 62 (30-82) | 59 (32-80) |

Sex, n (%)

| | | |
|---|---|---|
| Female | 76 (58) | 31 (57) |
| Male | 56 (42) | 23 (43) |

Race, n (%)

| | | |
|---|---|---|
| Asian | 73 (55) | 29 (54) |
| White | 39 (30) | 15 (28) |
| Missing | 20 (15) | 10 (19) |

ECOG PS, n (%)

| | | |
|---|---|---|
| 0 | 45 (34) | 22 (41) |
| 1 | 87 (66) | 32 (59) |
| Tobacco use, n (%) | | |
| Never | 81 (61) | 30 (56) |
| Current | 2 (2) | 0 |
| Former | 49 (37) | 24 (44) |

The safety profile in patients with brain metastases was similar to the overall population. The majority of TRAEs were mild and manageable. Rates of grade ≥3 EGFR-related TRAEs were low. Rates of dose reductions and discontinuations due to AEs were also low. There were no fatal TRAEs. TRAEs observed in Phase Ib are reported in Table 5.

TABLE 5
Treatment-related adverse events (TRAEs) in Phase
Ib. TRAEs that occurred in ≥8 patients are shown.

| | All patients | | Brain metastases | |
|---|---|---|---|---|
| | (N = 132) | | (n = 54) | |
| TRAEs* (%) | Any | Grade ≥3 | Any | Grade ≥3 |
| Any TRAE | 126 (96) | 24 (18) | 53 (98) | 12 (22) |
| Diarrhea | 73 (55) | 2 (2) | 29 (54) | 1 (2) |
| Rash^{‡} | 34 (26) | 0 | 12 (22) | 0 |
| ALT increased | 30 (23) | 12 (9) | 14 (26) | 6 (11) |
| AST increased | 30 (23) | 8 (6) | 14 (26) | 3 (6) |
| Anemia | 18 (14) | 0 | 8 (15) | 0 |
| AEs leading to | 4 (3) | 2 (2) | 2 (4) | 1 (2) |
| discontinuation | | | | |
| AEs leading to | 14 (11) | 8 (6) | 6 (11) | 3 (6) |
| dose reduction | | | | |

In all 132 patients treated with zongertinib in cohort 1 of Phase Ib, the confirmed ORR (RECIST v1.1) was 72% (3 CR, 92 PR) and the confirmed DCR was 85%.

Of the 54 evaluable patients with brain metastases, the confirmed ORR (RECIST v1.1) was 70% (1 CR, 37 PR) and the confirmed DCR was 94%.

Of the 78 evaluable patients without brain metastases, the confirmed ORR (RECIST v1.1) was 73% (2 CR, 55 PR) and the confirmed DCR was 96%.

The response rate was very similar in patients with, and those without, brain metastases. Zongertinib showed clinically meaningful activity in patients with pre-treated HER2-mutant NSCLC, regardless of the presence of brain metastases.

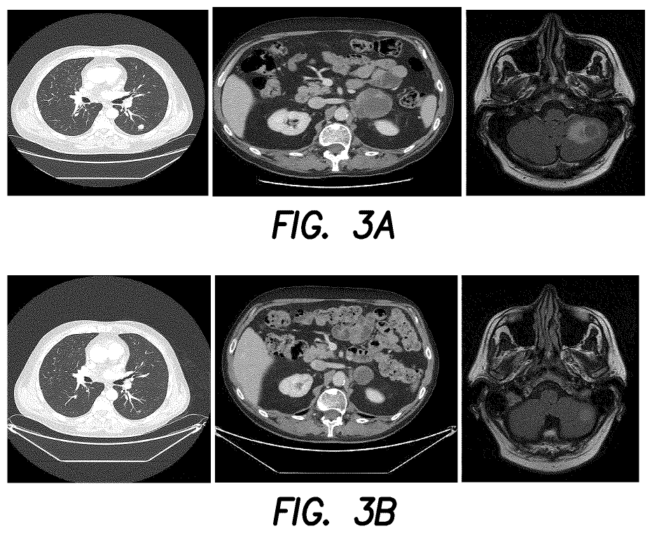

Further efficacy results for patients with brain metastases in cohort 1 of Phase Ib are reported in Tables 6a and 6b and in FIG. 3.

TABLE 6a
Efficacy in patients with brain metastases of cohort 1 of Phase Ib, divided by dose.

| | RECIST v1.1 central | | | | | | RANO-BM central | | | | | |
|---|---|---|---|---|---|---|---|---|---|---|---|---|
| | review | | | | | | review | | | | | |
| | 120 mg | | 240 mg | | Total | | 120 mg | | 240 mg | | Total | |
| | (n = 28) | | (n = 26) | | (N = 54) | | (n = 27) | | (n = 25) | | (N = 52) | |
| Overall Response Rate - | 19 | (68) | 19 | (73) | 38 | (70) | 9 | (33) | 10 | (40) | 19 | (37) |
| ORR, n (%) | | | | | | | | | | | | |
| 95% Confidence Interval - CI | 49-82 | | 54-86 | | 57-81 | | 19-52 | | 23-59 | | 25-50 | |
| Complete Response - CR, n | 0 | (0.0) | 1 | (4) | 1 | (2) | 4 | (15) | 5 | (20) | 9 | (17) |
| (%) | | | | | | | | | | | | |
| Partial Response - PR, n (%) | 19 | (68) | 18 | (69) | 37 | (69) | 5 | (19) | 5 | (20) | 10 | (19) |
| Disease Control Rate - | 25 | (89) | 26 | (100) | 51 | (94) | 20 | (74) | 23 | (92) | 43 | (83) |
| DCR, n (%) | | | | | | | | | | | | |
| 95% Confidence Interval - CI | — | | — | | — | | 55-87 | | 75-98 | | 70-91 | |
| Stable Disease - SD, n (%) | 6 | (22) | 7 | (27) | 13 | (24) | 11 | (41) | 13 | (52) | 24 | (46) |
| Progressive Disease - PD, n | 3 | (11) | 0 | (0) | 3 | (6) | 2 | (7) | 1 | (4) | 3 | (6) |
| (%) | | | | | | | | | | | | |
| Not evaluable, n (%) | 0 | (0) | 0 | (0) | 0 | (0) | 5 | (19) | 1 | (4) | 6 | (12) |

TABLE 6b
Efficacy in patients with brain metastases of cohort 1
of Phase Ib, focused on patients with brain metastases.

| Confirmed BOR | Brain | No brain | |
|---|---|---|---|
| (RECIST v1.1) | metastases | metastases | All patients |
| BICR | N = 54 | n = 78 | N = 132 |
| ORR, % | 70 | 71 | 72 |
| 95% CI | 57-81 | 62-82 | 64-79 |
| CR, n (%) | 1 (2) | 2 (3) | 3 (2) |
| PR, n (%) | 37 (69) | 55 (71) | 92 (70) |
| DCR, % | 94 | 96 | 95 |
| SD, n (%) | 13 (24) | 18 (23) | 31 (24) |
| PD, n (%) | 3 (6) | 0 (0) | 3 (2) |
| Not evaluable, n (%) | 0 (0) | 3 (4) | 3 (2) |

Zongertinib was active against CNS lesions. It demonstrated a manageable safety profile and clinically meaningful activity in pre-treated HER2-mutant NSCLC patients with asymptomatic brain metastases. In all patients eligible for RANO-BM assessment, the ORR and DCR were 37% and 83%, respectively (independent review). Nine patients (17.3%) had CR. These data show encouraging preliminary intracranial activity with zongertinib at both dose levels. In patients eligible for RANO-BM assessment, any target or non-target CNS lesion was entered for RANO-BM assessment at baseline.

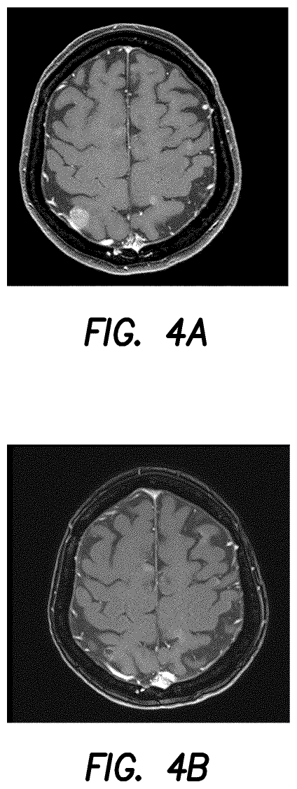

FIG. 3 shows a case study of a patient with baseline brain metastases from cohort 1 of Phase Ib. The patient was a 51 years-old male, former smoker. He was diagnosed with stage IV NSCLC with a HER2 exon 20 insertion (p.Y772_A775dup) and underwent 4 lines of non-HER2 targeted therapy. At baseline, there were lung, adrenal and asymptomatic, untreated brain metastases. Treatment with 240 mg QD zongertinib was then started. A partial response was observed after 6 weeks, including an effect in brain metastases. Treatment is ongoing. FIG. 4 shows another case study of a patient with a baseline brain metastasis from cohort 1 of Phase Ib. The patient was a 63 years-old female. She was diagnosed with stage IV NSCLC with a HER2 exon 20 insertion (p.Y772_A775dup). After 6 months of carboplatin-pemetrexed-pembrolizumab treatment, there was progressive disease. She received 240 mg QD zongertinib as second-line treatment. The duration of response in brain by RANO-BM was of 6 months (since discontinued treatment due to PD).

### Example 4—Updated Results from Cohort 1 of the Dose Expansion Part (Phase Ib)

This example is an update of Example 3, with a data-cutoff date of Nov. 29, 2024, which is approximately 6 months after the data-cutoff date of Example 3. The same number of 132 patients was treated in cohort 1.

The updated baseline characteristics of enrolled patients are reported in Table 7. More than one third (37%) of these patients had brain metastases at baseline. Several baseline characteristics remain as reported in Tables 4a and/or 4b. Additional characteristics are reported in Table 7.

TABLE 7
Updated baseline characteristics of patients
enrolled in cohort 1 of Phase Ib

| | Cohort 1 | | Cohort 1 | |
|---|---|---|---|---|
| | 120 mg | | 240 mg | |
| Characteristic | (N = 75) | | (N = 57) | |
| Median age - yr (range) | 62 | (30-80) | 62 | (36-82) |

Sex - no. (%)

| | | | | |
|---|---|---|---|---|
| Female | 51 | (68) | 25 | (44) |
| Male | 24 | (32) | 32 | (56) |

Race - no. (%)†

| | | | | |
|---|---|---|---|---|
| Asian | 40 | (53) | 33 | (58) |
| White | 24 | (32) | 15 | (26) |
| Missing‡ | 11 | (15) | 9 | (16) |

ECOG PS - no. (%)

| | | | | |
|---|---|---|---|---|
| 0 | 28 | (37) | 17 | (30) |
| 1 | 47 | (63) | 40 | (70) |

Tobacco use - no. (%)

| | | | | |
|---|---|---|---|---|
| Never | 49 | (65) | 32 | (56) |
| Current | 2 | (3) | 0 | (0) |
| Former | 24 | (32) | 25 | (44) |

No. of previous lines of systemic anticancer treatment - no. (%)

| | | | | |
|---|---|---|---|---|
| 0 | 3 | (4)§ | 2 | (4)§ |
| 1 | 43 | (57) | 28 | (49) |
| 2 | 12 | (16) | 15 | (26) |
| 3-4 | 13 | (17) | 8 | (14) |
| 5-6 | 2 | (3) | 3 | (5) |
| ≥7 | 2 | (3) | 1 | (2) |

Previous systemic therapy - no. (%)

| Chemotherapy | 71 | (95) | — | |
|---|---|---|---|---|
| Antibody therapy | 18 | (24) | — | |
| Immunotherapy | 52 | (69) | — | |
| Tyrosine kinase inhibitor | 2 | (3) | — | |
| therapy | | | | |
| Previous HER2-targeted therapy - | 7 | (9) | 5 | (9) |
| no. (%)¶ | | | | |
| Previous immune checkpoint | 57 | (76) | 43 | (75) |
| inhibitor therapy - no. (%) | | | | |

| | | | |
|---|---|---|---|
| Previous brain radiotherapy - no. (%) | 13 | (17) | — |

Site of metastases at screening - no. (%)

| | | | | |
|---|---|---|---|---|
| Brain | 28 | (37) | 25 | (44) |
| Liver | 17 | (23) | 15 | (26) |

Method used for HER2 sequencing - no. (%)

| | | | | |
|---|---|---|---|---|
| Next-generation sequencing | 70 | (93) | 51 | (90) |
| Polymerase chain reaction | 3 | (4) | 4 | (7) |
| Other | 1 | (1) | 2 | (4) |

| | | | |
|---|---|---|---|
| Missing | 1 | (1) | — |

HER2 TKD mutation type - no. (%)

| A775_G776insYVMA | 43 | (57) | 30 | (53) |
|---|---|---|---|---|
| P780_Y781insGSP | 8 | (11) | 4 | (7) |
| A775_G776insYVMA, Other | 5 | (7) | 4 | (7) |
| G776 > VC | 3 | (4) | 3 | (5) |
| L755P | 2 | (3) | 3 | (5) |
| G776V | 2 | (3) | — | |
| G776 > LC | — | | 2 | (4) |
| Other | 12 | (16) | 11 | (19) |

†Race was reported by the investigators at screening.
‡Data on race were missing because of legal requirements.
§Patients had received previous treatment in an adjuvant context only but within 6 months before initiating zongertinib; therefore, these patients were considered as having been previously treated in the context of advanced or metastatic disease in accordance with the protocol.
¶The exclusion criterion regarding previous HER2 therapy was added during a protocol amendment after initiation of recruitment.

Treatment-related adverse events (all/grade ≥3) were reported in 100%/21% of patients with brain metastases and 96%/15% of patients without brain metastases.

In cohort 1, adverse events that occurred during the treatment period were reported in all patients who received zongertinib at a dose of 120 mg. Drug-related adverse events were reported in 73 patients (97%) (Table 8), and grade 3 or higher drug-related adverse events were reported in 13 patients (17%), the most common being alanine aminotransferase level (8%) and increased aspartate aminotransferase level (5%). One patient (1%) had grade 4 drug-related adverse events (increased alanine aminotransferase level, hypertransaminasemia, and suspected drug-induced liver injury). A total of 7 patients (9%) had fatal adverse events; none were considered by the investigators to be related to zongertinib (malignant neoplasm progression in 5 patients, disease progression in 1 patient and acute respiratory failure in 1 patient). No cases of drug-related interstitial lung disease or toxic effects related to interstitial lung disease were reported. Overall, 42 patients (56%) had drug-related diarrhea; 48% had grade 1, 7% had grade 2, and 1 (1%) had grade 3. All cases of drug-related rash (33%) were grade 1 (24%) or grade 2 (9%). Adverse events leading to dose reduction of zongertinib were reported in 5 patients (7%), and adverse events leading to discontinuation of zongertinib were reported in 2 patients (3%). In cohort 1, the incidence of grade 3 or higher drug-related adverse events was slightly higher among patients treated with zongertinib at a dose of 240 mg than those treated at a dose of 120 mg during the randomization phase (25% vs. 22%), as was the incidence of grade 3 or higher drug-related diarrhea (5% vs. 2%) and the incidence of adverse events of any grade leading to dose reduction (22% vs. 7%).

TABLE 8
Safety Summary and Most Common Drug-Related Adverse Events
in Patients Treated with Zongertinib 120 mg in Cohort 1.

| Adverse events that | Event | | | | | |
|---|---|---|---|---|---|---|
| occurred during the | All | Grade 1 | Grade 2 | Grade 3 | Grade 4 | Grade 5 |
| treatment period | number of patients (%) | | | | | |
| Any | 75 (100) | 11 (15) | 29 (39) | 26 (35) | 2 (3) | 7 (9) |
| Led to dose | 5 (7) | 1 (1) | 0 | 3 (4) | 1 (1) | 0 |
| reduction* | | | | | | |
| Led to treatment | 2 (3) | 1 (1) | 0 | 1 (1) | 0 | 0 |
| discontinuation† | | | | | | |
| Serious drug-related | 3 (4) | 0 | 0 | 2 (3) | 1 (1) | 0 |
| adverse events‡ | | | | | | |
| Any drug-related adverse | 73 (97) | 27 (36) | 33 (44) | 12 (16) | 1 (1) | 0 |
| event § | | | | | | |
| Diarrhea¶ | 42 (56) | 36 (48) | 5 (7) | 1 (1) | 0 | 0 |
| Rash ∥ | 25 (33) | 18(24) | 7(9) | 0 | 0 | 0 |
| Aspartate | 18 (24) | 11 (15) | 3 (4) | 4 (5) | 0 | 0 |
| aminotransferase | | | | | | |
| increased | | | | | | |
| Alanine | 16 (21) | 9 (12) | 1 (1) | 5 (7) | 1 (1) | 0 |
| aminotransferase | | | | | | |
| increased | | | | | | |
| Nausea | 11 (15) | 10 (13) | 1 (1) | 0 | 0 | 0 |
| Dry skin | 11 (15) | 11(15) | 0 | 0 | 0 | 0 |
| Pruritus | 10 (13) | 9 (12) | 1 (1) | 0 | 0 | 0 |
| White-cell count | 10 (13) | 5 (7) | 5 (7) | 0 | 0 | 0 |
| decreased | | | | | | |
| Anemia | 9 (12) | 6 (8) | 3 (4) | 0 | 0 | 0 |
| Neutrophil count | 9 (12) | 3 (4) | 5 (7) | 1 (1) | 0 | 0 |
| decreased | | | | | | |
| Nail disorder | 8 (11) | 8 (11) | 0 | 0 | 0 | 0 |

*Adverse events included aspartate aminotransferase increased (2 patients), alanine aminotransferase increased (2 patients), neutrophil count decreased (1 patient), hypertransaminasemia (1 patient), suspected drug-induced liver injury (1 patient), blood creatine phosphokinase increased (1 patient), and gamma-glutamyltransferase increased (1 patient).
†Adverse events included alanine aminotransferase increased, aspartate aminotransferase increased, blood alkaline phosphokinase increased, gamma-glutamyltransferase increased, and pyrexia (1 patient each).
‡Events included alanine aminotransferase increased (3 patients), aspartate aminotransferase increased (2 patients), hypertransaminasemia (1 patient), and suspected drug-induced liver injury (1 patient).
§ Drug-related adverse events were assessed by the investigator; those reported in more than 10% of patients are reflected.
¶The grouped term diarrhea includes the preferred terms diarrhea and intestinal transit time decreased.
∥ The grouped term rash includes the preferred terms dermatitis, dermatitis acneiform, dermatitis allergic, rash, rash erythematous, rash maculopapular, and rash pustular.

At the updated data-cutoff date of this Example, the median follow-up was 11.3 months (95% CI, 10.2 to 12.3). At this time, 53 patients (71%; 95% CI, 60 to 80; P value <0.001) treated with 120 mg had a confirmed objective response: 5 patients (7%) had a complete response, and 48 patients (64%) had a partial response. Of the 53 patients who had a response, 21 (40%) had an ongoing response at the data-cutoff date. The confirmed DCR was 96% (95% CI, 89 to 99), with 72 patients achieving disease control (19 SD and 3 PD) The median duration of response was 14.1 months (95% CI, 6.9 to not evaluable). The median progression-free survival was 12.4 months (95% CI, 8.2 to not evaluable).

A total of 28 patients had brain metastases at screening, of whom 18 (64%; 95% CI, 46 to 79) had a confirmed systemic objective response according to RECIST, version 1.1; of the 28 patients, 1 (4%) had a complete response and 17 (61%) had a partial response. As a comparison, in the 47 patients without brain metastases at screening, the confirmed systemic objective response according to RECIST, version 1.1 was 74% (95% CI, 61-85).

Among the 27 patients who were eligible for assessment according to RANO-BM criteria, the confirmed intracranial objective response was 41% (95% CI, 25 to 59), including 4 (15%) patients with an intracranial complete response. The intracranial disease control rate was 81% (95% CI: 63-92) (Table 9).

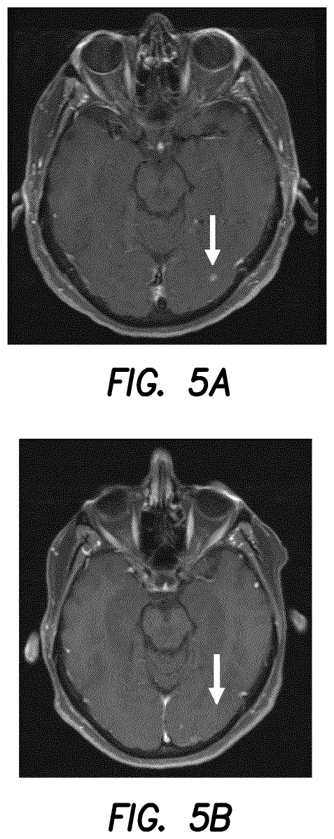

This is illustrated by a patient treated with zongertinib 120 mg QD in FIG. 5, who had disappearance of a number of CNS lesions (e.g. see arrow in FIG. 5).

Of the 18 RANO-BM eligible patients who had not received brain radiotherapy prior to treatment, 2 had a complete response (CR) and 3 had a partial response (PR). Of the 9 RANO-BM eligible patients who had received brain radiotherapy prior to treatment, 2 patients had a CR and 4 had a PR.

Among all 75 patients in cohort 1, the median best percentage change from baseline in the sum of the diameters of target lesions was −43% (range, −100 to 22). Of the 75 patients, 42 had disease progression: 8 (11%) had isolated central nervous system progression, 26 (35%) had isolated non-central nervous system progression, 4 (5%) had simultaneous central nervous system and non-central nervous system progression (according to investigator assessment), and 4 (5%) had an unknown site of progression. Among the 55 patients in cohort 1 who received zongertinib at a dose of 240 mg during the randomization phase (two patients were recruited before randomization and were not included in the analysis), the confirmed objective response was 84% (95% CI, 72 to 91), the median duration of response was 9.7 months (95% CI, 8.3 to 11.0), and the median progression-free survival was 10.9 months (95% CI, 9.6 to 12.4). Among the 24 patients who were eligible for assessment according to RANO-BM criteria and received zongertinib at a dose of 240 mg during the randomization phase, the confirmed intracranial objective response was 42% (95% CI, 25 to 61).

TABLE 9
Intracranial Response to Zongertinib 120 mg, as Assessed
by Blinded Independent Central Review in Patients with
Central Nervous System Lesions at Baseline in Cohort 1.

| | Cohort 1 | |
|---|---|---|
| Response Assessment* | 120 mg | |
| RANO-BM | (N = 27) | |
| Objective response - no. (%) | 11 | (41) |
| 95% CI | 25-59 | |
| Complete response - no. (%) | 4 | (15) |
| Partial response - no. (%) | 7 | (26) |
| Disease control - no. (%) | 22 | (81) |
| 95% CI | 63-92 | |
| Stable disease - no. (%) | 11 | (41) |
| Progressive disease - no. (%) | 2 | (7) |
| Not evaluable - no. (%) | 3 | (11) |

*In patients eligible for RANO-BM assessment only (patients where any target or non-target lesion was entered for RANO-BM assessment at baseline)

Zongertinib demonstrated intracranial activity in patients with brain metastases. The systemic objective response rate according to RECIST, version 1.1, was similar in the subgroup of patients with brain metastases at screening who were treated with zongertinib at a dose of 120 mg (64%) and the overall cohort 1 population treated at the same dose (71%), which suggests that zongertinib may be a promising treatment option in patients with brain metastases.

### Example 5—Preliminary Results from Cohorts 3 and 5 of the Dose Expansion Part (Phase Ib)

This Example reports preliminary results from cohorts 3 and 5 of the dose expansion part (Phase Ib).

39 patients were treated in cohort 5 and 25 patients were treated in cohort 3. The baseline characteristics of patients treated with zongertinib at a dose of 120 mg in cohort 5 (31 patients) and cohort 3 (20 patients) are shown in Table 10.

TABLE 10
Baseline Demographic and Disease Characteristics of Patients
Treated with Zongertinib 120 mg in Cohorts 3 and 5.

| | Cohort 3* | | Cohort 5 | |
|---|---|---|---|---|
| Characteristic | (N = 20) | | (N = 31) | |
| Median age - yr (range) | 65 | (27-77) | 61 | (31-85) |

Sex - no. (%)

| | | | | |
|---|---|---|---|---|
| Female | 9 | (45) | 21 | (68) |
| Male | 11 | (55) | 10 | (32) |

Race - no. (%)

| | | | | |
|---|---|---|---|---|
| Asian | 9 | (45) | 11 | (35) |
| White | 8 | (40) | 14 | (45) |
| Missing^{†} | 3 | (15) | 6 | (19) |

Eastern Cooperative Oncology Group Performance Status - no. (%)

| | | | | |
|---|---|---|---|---|
| 0 | 4 | (20) | 7 | (23) |
| 1 | 16 | (80) | 24 | (77) |

Tobacco use - no. (%)

| | | | | |
|---|---|---|---|---|
| Never | 10 | (50) | 20 | (65) |
| Former | 9 | (45) | 11 | (35) |

| | | | |
|---|---|---|---|
| Missing | 1 | (5) | 0 |

Number of previous lines of systemic anticancer treatment - no. (%)

| 1 | 8 | (40) | 5 | (16) |
|---|---|---|---|---|
| 2 | 9 | (45) | 13 | (42) |
| 3-4 | 3 | (15) | 10 | (32) |
| 5-6 | 0 | | 2 | (6) |
| ≥7 | 0 | | 1 | (3) |
| Prior HER2-targeted therapy - no. (%) | 1 | (5) | 30 | (97) |
| T-DXd | 1 | (5) | 22 | (71) |
| T-DM1 | 0 | | 3 | (10) |
| Prior immune checkpoint inhibitor | 17 | (85) | 24 | (77) |
| therapy - no. (%) | | | | |

Site of metastases at screening - no. (%)

| | | | | |
|---|---|---|---|---|
| Brain | 8 | (40) | 23 | (74) |
| Liver | 6 | (30) | 11 | (35) |

Method used for HER2 sequencing^{‡} - no. (%)

| Next-generation sequencing | 19 | (95) | 29 | (94) |
|---|---|---|---|---|
| Polymerase chain reaction | 0 | | 1 | (3) |
| Other | 1 | (5) | 1 | (3) |

HER2 TKD mutation type - no. (%)

| | | | |
|---|---|---|---|
| A775_G776insYVMA | 0 | 18 | (58) |
| P780_Y781insGSP | 0 | 3 | (10) |
| G776 > VC | 0 | 3 | (10) |
| Other | 0 | 7 | (23) |

HER2 non-TKD mutation type - no. (%)

| | | | |
|---|---|---|---|
| S310F^{§} | 6 | (30) | 0 |
| V659E | 6 | (30) | 0 |
| S310Y | 4 | (20) | 0 |
| P1199S | 1 | (5) | 0 |
| D277Y | 1 | (5) | 0 |
| S113F | 1 | (5) | 0 |
| G660D | 1 | (5) | 0 |

*Non-squamous non-TKD mutant only.
^{†}*Due to legal requirements.
^{‡}Local HER2 testing.
^{§†}One patient had a co-amplification.

The safety profile of zongertinib in cohorts 5 and 3 is shown in Table 11. Grade 3 or higher drug-related adverse events were reported in 1 patient (3%) in cohort 5 and in 5 patients (25%) in cohort 3.

TABLE 11
Safety Summary and Most Common Drug-Related Adverse Events in
Patients Treated with Zongertinib 120 mg in Cohorts 3 and 5.

| | Cohort 3 | | | | Cohort 5 | | | |
|---|---|---|---|---|---|---|---|---|
| | (N = 20) | | | | (N = 31) | | | |
| | All | | Grade ≥3 | | All | | Grade ≥3 | |
| Event | no. (%) | | | | | | | |
| Any treatment-emergent adverse event | 20 | (100) | 7 | (35) | 31 | (100) | 16 | (52) |
| Treatment-emergent adverse event leading | 4 | (20) | 3 | (15) | 2 | (6) | 0 | |
| to dose reduction | | | | | | | | |
| Treatment-emergent adverse event leading | 2 | (10) | 2 | (10) | 2 | (6) | 2 | (6) |
| to discontinuation | | | | | | | | |
| Serious drug-related adverse events | 3 | (15) | 3 | (15) | 0 | | 0 | |
| Any drug-related adverse event* | 16 | (80) | 5 | (25) | 24 | (77) | 1 | (3) |

| Diarrhea^{†} | 9 | (45) | 1 | (5) | 10 | (32) | 0 |
|---|---|---|---|---|---|---|---|
| Pruritus | 5 | (25) | 0 | | 2 | (6) | 0 |
| Rash^{‡} | 3 | (15) | 0 | | 6 | (19) | 0 |
| Blood creatinine increased | 3 | (15) | 0 | | 1 | (3) | 0 |
| Hypokalemia | 3 | (15) | 0 | | 0 | | 0 |
| Aspartate aminotransferase increased | 2 | (10) | 0 | | 4 | (13) | 0 |
| Alanine aminotransferase increased | 2 | (10) | 1 | (5) | 3 | (10) | 0 |

| Asthenia | 2 | (10) | 0 | 1 | (3) | 0 | |
|---|---|---|---|---|---|---|---|
| Dry skin | 2 | (10) | 0 | 1 | (3) | 0 | |
| Anemia | 2 | (10) | 0 | 2 | (6) | 0 | |
| Neutrophil count decreased | 2 | (10) | 0 | 1 | (3) | 0 | |
| Nausea | 1 | (5) | 0 | 4 | (13) | 1 | (3) |

| | | | | | | |
|---|---|---|---|---|---|---|
| Lipase increased | 2 | (10) | 0 | 1 | (3) | 0 |

| | | | | | | |
|---|---|---|---|---|---|---|
| Drug-induced liver injury | 2 | (10) | 2 | (10) | 0 | 0 |

| | | | | | |
|---|---|---|---|---|---|
| Ejection fraction decreased | 0 | 0 | 4 | (13) | 0 |
| Fatigue | 0 | 0 | 3 | (10) | 0 |

Data cut-off: Nov. 29, 2024.
*As assessed by the investigator; those reported in ≥10% of patients in any cohort are shown.
^{†}Grouped term including preferred terms diarrhea only (Cohort 3) and diarrhea, gastroenteritis, and enteritis (Cohort 5).
^{‡}Grouped term including preferred terms rash, dermatitis and rash maculo-papular (Cohort 3), and dermatitis acneiform, post procedural erythema and rash (Cohort 5).

In the primary analysis of cohort 5 (August 2024), the confirmed objective response among patients with NSCLC with a mutation in the tyrosine kinase domain who had been previously treated with a HER2-directed antibody-drug conjugate was 42% (95% CI, 26 to 59; P=0.01 against the ≤25% benchmark). The data on median duration of response and progression-free survival were not yet mature. By November 2024, the median duration of follow-up was 6.8 months (95% CI, 5.5 to 8.5). At this time, 15 patients (48%; 95% CI, 32 to 65) had a confirmed objective response (Table 12). Of the 22 patients who had previously received trastuzumab deruxtecan, 9 had an objective response (41%; 95% CI, 23 to 61). Overall, the median duration of response was 5.3 months (95% CI, 2.8 to not evaluable). The median progression-free survival was 6.8 months (95% CI, 5.4 to not evaluable).

In the exploratory cohort 3, a total of 6 patients with NSCLC with non-tyrosine kinase domain mutations (30%; 95% CI, 15 to 52) had a confirmed objective response. Responses were observed across non-tyrosine kinase domain mutation types (Table 13). The median duration of response and progression-free survival were not yet mature at the data-cutoff date.

TABLE 12
Response to Zongertinib 120 mg as Assessed by
Investigator Review in Cohort 3 and by Blinded
Independent Central Review in Cohort 5.

| Response assessment | Cohort 3 | Cohort 5 |
|---|---|---|
| RECIST, version 1.1 | (N = 20) | (N = 31) |
| Objective response - no. (%) | 6 (30) | 15 (48) |
| 95% CI | 15-52 | 32-65 |
| Complete response - no. (%) | 0 | 1 (3) |
| Partial response - no. (%) | 6 (30) | 14 (45) |
| Disease control - no. (%) | 13 (65) | 30 (97) |
| 95% CI | 43-82 | 84-99 |
| Stable disease - no. (%) | 7 (35) | 15 (48) |
| Progressive disease - no. (%) | 6 (30) | 0 |
| Not evaluable - no. (%) | 1 (5) | 1 (3) |

Data cut-off: Nov. 29, 2024. CI denotes confidence interval, and RECIST Response Evaluation Criteria in Solid Tumors.

TABLE 13
Response to Zongertinib 120 mg by Non-TKD Mutation Type
as Assessed by Investigator Review in Cohort 3.
Cohort 3 (N = 20)

| | | | | Objective | |
|---|---|---|---|---|---|
| | | | Activating | response - | |
| HER2 non-TKD mutation type - no. (%) | | | mutation | no. (%) | |
| Extracellular domain | 12 | (60) | — | 3 | (25) |
| S310F* | 6 | (30) | Known | 3 | (50) |
| S310Y | 4 | (20) | Known | 0 | |
| D277Y | 1 | (5) | Known | 0 | |
| S113F | 1 | (5) | Unknown | 0 | |
| Transmembrane domain | 7 | (35) | — | 3 | (43) |
| V659E | 6 | (30) | Known | 3 | (50) |

| | | | | |
|---|---|---|---|---|
| G660D | 1 | (5) | Likely | 0 |
| Intracellular domain | 1 | (5) | — | 0 |
| P1199S | 1 | (5) | Unknown | 0 |

Data cut-off: Nov. 29, 2024.
*One patient had a co-amplification.

### Example 6—Updated Results from the Dose Escalation Part (Phase Ia)

Similarly to Example 2, this example reports the results of the dose escalation part (Phase Ia) of Example 1, in particular intracranial efficacy per RANO-BM. The data-cutoff date is Mar. 26, 2025, which is approximately 10 months after the data-cutoff date of Example 2. By this time, 124 patients with advanced, unresectable and/or metastatic pre-treated solid tumors with an aberration of the HER2 gene were enrolled. Around one quarter had asymptomatic brain metastases at baseline (30 patients or 24.2% out of the total 124 patients). These patients were heavily pretreated (>2 lines of previous therapy: 56.5%). Further baseline characteristics of enrolled patients are reported in Table 14.

TABLE 14
Baseline characteristics of patients enrolled in Phase Ia.

| | All patients | |
|---|---|---|
| Characteristic | (n = 124) | |
| Median age, years (range) | 60 | (31-81) |
| Female, n (%) | 64 | (51.6) |
| Country of participation, n (%) | | |
| USA | 32 | (25.8) |
| Netherlands | 31 | (25.0) |
| Japan | 54 | (43.5) |
| China | 7 | (5.6) |
| Prior lines of therapy, n (%) | | |
| ≤2 | 54 | (43.5) |
| >2 | 70 | (56.5) |
| Tumor type, n (%) | | |
| NSCLC | 53 | (42.7) |
| Colorectal cancer | 17 | (13.7) |
| Breast cancer | 16 | (12.9) |
| Esophageal cancer | 7 | (5.6) |
| Other tumors** | 31 | (25) |
| HER2m+* | 65 | (52.4) |
| Brain metastases at baseline | 30 | (24.2) |

*HER2 mutation-positive. Patients without HER2m+ tumors had other HER2 aberrations, as defined in the protocol of Example 1.
**other tumors include: biliary tree, bladder, cancers of the gastrointestinal tract, cancers of the mediastinum, cervix, endometrial carcinoma, genitourinary system, head and neck cancers, ovary, pancreas, small intestine, stomach, uterine, vulva, and cancer types not specified further.

Systemic response rates per RECIST 1.1 were similar among all patients and those with brain metastases. Of the 124 patients in Phase Ia, the overall response rate (ORR) by RECIST v1.1 was 41% and the disease control rate (DCR) was 86%. 53 of these patients had NSCLC. For them, the ORR was 47.2% and the DCR 94%. The median DoR in all responders was 9.7 months with a (95% confidence interval, CI, 6.9—not calculated, NC). The median PFS in all 124 patients were 8.0 months (BID, 95% CI: 2.8-29.0) and 7.0 months (QD, 95% CI: 5.5-9.9). Of the 30 patients with brain metastases, according to the systemic response assessment based on RECIST v1.1, the ORR and DCR in non-CNS lesions were 33% and 87%, respectively.

Efficacy in brain lesions was assessed per RANO-BM by the investigator. Only 6 of the 30 patients had measurable lesions in the brain at baseline. In this context, “measurable” refers to lesions for which baseline and post-baseline RANO-BM longest diameter measurements of lesions were reported. The remaining patients had non-measurable brain lesions by RANO-BM. According to the RANO-BM criteria, patients that only have non-measurable brain lesions can have an objective response of PD, SD, CR or NE, but not of PR. For patients with measurable brain lesions, partial response is also possible.

Among the 30 patients with brain metastases at baseline, 2 (reported primary tumor classification: non-small cell lung cancer, cancers of the breast) showed a confirmed complete response in brain lesions per RANO-BM, and 3 (reported primary tumor classifications: non-small cell lung cancer, head and neck cancer, cancers of the vulva) showed a confirmed partial response in brain lesions per RANO-BM. 18 patients (tumor types: 14 patients with NSCLC, 2 patients cancers of the lung, 1 patient ovary cancer, 1 patient cancers of the breast) showed stable disease in the brain per RANO-BM. This corresponds to a confirmed ORR of 16.7%, and a confirmed disease control rate of 76.7%.

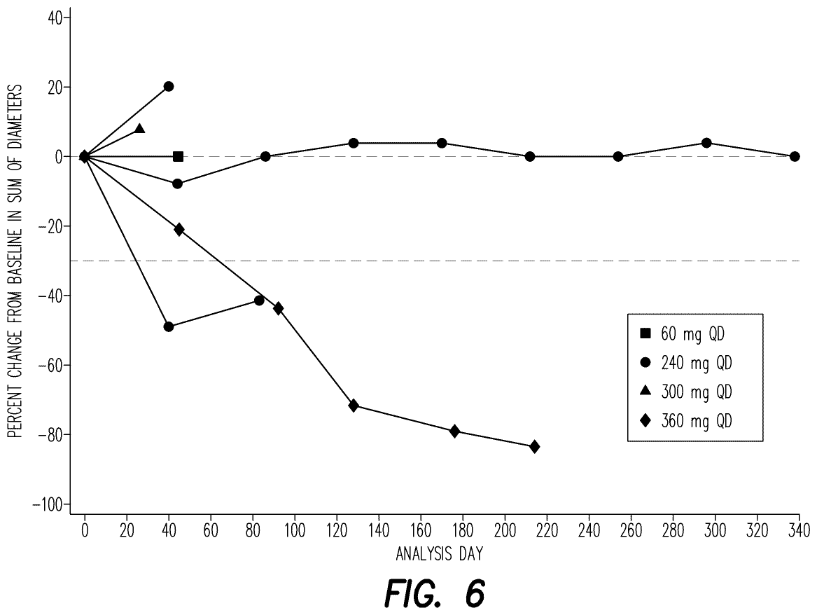

For the 6 patients with measurable brain metastases at baseline, CNS target lesion measurements per RANO-BM (investigator's assessment) are shown in FIG. 6. The mean intracranial tumor shrinkage from baseline was −18.7% and up to −83.6% tumor shrinkage in brain lesions was reported.

The data of this Example confirm the intracranial efficacy of zongertinib in the patient population of the Phase Ia part of the study of Example 1.

### Example 7—Further Updated Results from Cohort 1 of the Dose Expansion Part (Phase Ib)

This example is an update of Example 4, with a data-cutoff date of Mar. 26, 2025, which is approximately 4 months after the data-cutoff date of Example 4. The same number of 132 patients was treated in cohort 1.

The updated baseline characteristics of enrolled patients are reported in Table 15. Approximately 40% of the patients in Cohort 1 (53 out of the total 132) had brain metastases at baseline. Most baseline characteristics remain as reported in Example 4, in particular in Table 7. In Table 15, values that differ from Table 7 are in bold.

TABLE 15
Further updated baseline characteristics of
patients enrolled in cohort 1 of Phase Ib

| | Cohort 1 | | Cohort 1 | |
|---|---|---|---|---|
| | 120 mg | | 240 mg | |
| Characteristic | (N = 75) | | (N = 57) | |
| Median age - yr (range) | 62 | (30-80) | 62 | (36-82) |

Sex - no. (%)

| | | | | |
|---|---|---|---|---|
| Female | 51 | (68) | 25 | (44) |
| Male | 24 | (32) | 32 | (56) |

Race - no. (%)†

| | | | | |
|---|---|---|---|---|
| Asian | 40 | (53) | 33 | (58) |
| White | 29 | (39) | 15 | (26) |
| Missing‡ | 6 | (8) | 9 | (16) |

ECOG PS - no. (%)

| | | | | |
|---|---|---|---|---|
| 0 | 28 | (37) | 17 | (30) |
| 1 | 47 | (63) | 40 | (70) |

Tobacco use - no. (%)

| | | | | |
|---|---|---|---|---|
| Never | 49 | (65) | 32 | (56) |
| Current | 2 | (3) | 0 | (0) |
| Former | 24 | (32) | 25 | (44) |

No. of previous lines of systemic anticancer treatment - no. (%)

| | | | | |
|---|---|---|---|---|
| 0 | 3 | (4)§ | 2 | (4)§ |
| 1 | 43 | (57) | 28 | (49) |
| 2 | 12 | (16) | 15 | (26) |
| 3-4 | 13 | (17) | 8 | (14) |
| 5-6 | 2 | (3) | 3 | (5) |
| ≥7 | 2 | (3) | 1 | (2) |

Previous systemic therapy - no. (%)

| | | | | |
|---|---|---|---|---|
| Chemotherapy | 71 | (95) | 54 | (95) |
| Antibody therapy | 18 | (24) | 21 | (37) |
| Immunotherapy | 53 | (71) | 36 | (63) |
| Tyrosine kinase inhibitor | 2 | (3) | 3 | (5) |
| therapy | | | | |
| Previous HER2-targeted therapy - | 7 | (9) | 5 | (9) |
| no. (%)¶ | | | | |
| Previous immune checkpoint | 57 | (76) | 43 | (75) |
| inhibitor therapy - no. (%) | | | | |
| Previous brain radiotherapy - no. (%) | 11 | (15) | 11 | (19) |

Site of metastases at screening - no. (%)

| | | | | |
|---|---|---|---|---|
| Brain | 28 | (37) | 25 | (44) |
| Liver | 17 | (23) | 15 | (26) |

Method used for HER2 sequencing - no. (%)

| | | | | |
|---|---|---|---|---|
| Next-generation sequencing | 70 | (93) | 51 | (90) |
| Polymerase chain reaction | 3 | (4) | 4 | (7) |
| Other | 1 | (1) | 2 | (4) |

| | | | |
|---|---|---|---|
| Missing | 1 | (1) | — |

HER2 TKD mutation type - no. (%)

| | | | | |
|---|---|---|---|---|
| A775_G776insYVMA | 44 | (59) | 30 | (53) |
| P780_Y781insGSP | 8 | (11) | 4 | (7) |
| A775_G776insYVMA, Other | 5 | (7) | 4 | (7) |
| G776 > VC | 3 | (4) | 3 | (5) |
| L755P | 2 | (3) | 3 | (5) |
| G776V | 2 | (3) | 1 | (2) |
| G776 > LC | 1 | (1) | 2 | (4) |
| Other | 10 | (13) | 10 | (18) |

†, ‡, § and ¶ have the same meanings as defined in Table 7

Overall, safety data in patients with and without brain metastases at baseline were similar to the results reported in Example 4. Treatment-related adverse events (all/grade ≥3) were reported in 100%/21% of patients with brain metastases and 96%/17% of patients without brain metastases treated with 120 mg.

In cohort 1, adverse events that occurred during the treatment period were reported in all patients who received zongertinib at a dose of 120 mg. Drug-related adverse events were reported in 73 patients (97%), and grade 3 or higher drug-related adverse events were reported in 14 patients (19%), the most common being increased alanine aminotransferase level (8%) and increased aspartate aminotransferase level (5%). One patient (1%) had grade 4 drug-related adverse events (increased alanine aminotransferase level, hypertransaminasemia, and suspected drug-induced liver injury). A total of 8 patients (11%) had fatal adverse events; none were considered by the investigators to be related to zongertinib (malignant neoplasm progression in 6 patients, disease progression in 1 patient and acute respiratory failure in 1 patient). No cases of drug-related interstitial lung disease or toxic effects related to interstitial lung disease were reported. Overall, 41 patients (55%) had drug-related diarrhea; 48% had grade 1, 5% had grade 2, and 1 (1%) had grade 3. All cases of drug-related rash were grade 1 or grade 2. Adverse events leading to dose reduction of zongertinib were reported in 5 patients (7%), and adverse events leading to discontinuation of zongertinib were reported in 2 patients (3%).

Also at a dose of 240 mg, the safety profile of zongertinib remained manageable: only 3 of the 57 treated patients (5%) experienced AEs leading to discontinuation of treatment, and 21% (12 patients) experienced AEs leading to dose reduction. 12% (7 patients) experienced serious drug-related adverse events. Grade 3 or higher-drug related AEs were observed in 25% of patients (14 patients) at 240 mg. The most commonly observed drug-related AEs were mostly of mild grades (Grades 1 or 2) and their incidence at 240 mg (by preferred term) was comparable to 120 mg: the most common related AEs at 240 mg include diarrhoea (all grades 68%, Grade 3 or higher: 4%), alanine aminotransferase increased (all grades 30%, Grade 3 or higher: 11%), aspartate aminotransferase increased (all grades 25%, Grade 3 or higher: 7%) and rash (all grades 12%, Grade 3 or higher: 0%).

At the updated data-cutoff date of this Example, the median follow-up for progression-free survival was 16.4 months (95% CI, 13.8 to 17.8). At this time, 54 patients (72%; 95% CI, 61 to 81; P value <0.001) treated with 120 mg had a confirmed systemic objective response per RECIST 1.1 (central review): 5 patients (7%) had a complete response, and 49 patients (65%) had a partial response. Of the 54 patients who had a response, 19 (35%) had an ongoing response at the data-cutoff date. The confirmed DCR was 96% (95% CI, 89 to 99), with 72 patients achieving disease control (18 SD and 3 PD). The median duration of response was 14.1 months (95% CI, 8.3 to not evaluable). The median progression-free survival was 11.1 months (95% CI, 8.3 to 16.4).

A total of 28 patients treated with 120 mg zongertinib had brain metastases at screening, of whom 18 (64%; 95% CI, 46 to 79) had a confirmed systemic (i.e., including brain and non-brain lesions) objective response according to RECIST, version 1.1 assessed by central review; of the 28 patients, 1 (4%) had a complete response and 17 (61%) had a partial response. In these patients, the DCR was 89%. As a comparison, in the 47 patients without brain metastases at screening, the confirmed systemic objective response and DCR according to RECIST, version 1.1 were 77% (95% CI, 63-86) and 100%, respectively. Zongertinib elicited strong systemic responses, with similar benefit in patients with and without brain metastases.

Among the 28 patients with brain metastases treated with 120 mg zongertinib, the confirmed intracranial objective response rate by RANO-BM was 39% (95% CI, 24 to 58), including 4 (14%) patients with an intracranial complete response. The intracranial disease control rate was 79% (95% CI: 61-90) (Table 16).

In total 17 patients with brain metastases at baseline at 120 mg had not received brain radiotherapy prior to treatment, where 2 of these patients had a complete response (CR) and 3 had a partial response (PR) by RANO-BM. Of the 11 patients with brain metastases who had received brain radiotherapy prior to treatment, 2 patients had a CR and 4 had a PR by RANO-BM.

In 25 patients with brain metastases at baseline at 240 mg, 10 patients experienced an objective response per RANO-BM, leading to an ORR of 40% (95% CI: 23%-59%). Additionally, 13 patients showed stable disease, which corresponds to a disease control rate of 92%.

TABLE 16
Intracranial response to zongertinib 120 mg according to
RANO-BM, as assessed by blinded independent central review
in patients with CNS lesions at baseline in cohort 1

| | Cohort 1 | Cohort 1 |
|---|---|---|
| Response Assessment* | 120 mg | 240 mg |
| RANO-BM | (N = 28) | (N = 25) |
| Objective response - no. (%) | 11 (39) | 10 (40) |
| 95% CI | 24-58 | 23-59 |
| Complete response - no. (%) | 4 (14) | 4 (16) |
| Partial response - no. (%) | 7 (25) | 6 (24) |
| Disease control - no. (%) | 22 (79) | 23 (92) |
| 95% CI | 61-90 | 75-98 |
| Stable disease - no. (%) | 11 (39) | 13 (52) |
| Progressive disease - no. (%) | 4 (14) | 1 (4) |
| Not evaluable - no. (%) | 2 (7) | 1 (4) |

*In patients with any brain metastases at baseline

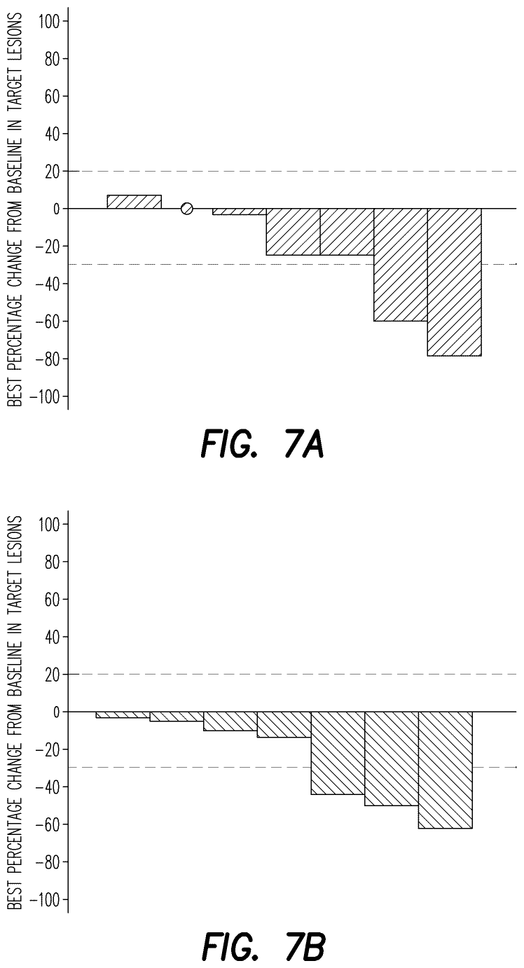

FIG. 7 shows the best tumor shrinkage in the brain per RANO-BM according to the investigator for patients with brain target lesions in cohort 1. Median tumor shrinkage was −25% for patients at 120 mg (greatest observed shrinkage: −78%) and −13% for patients at 240 mg (greatest observed shrinkage: −62%).

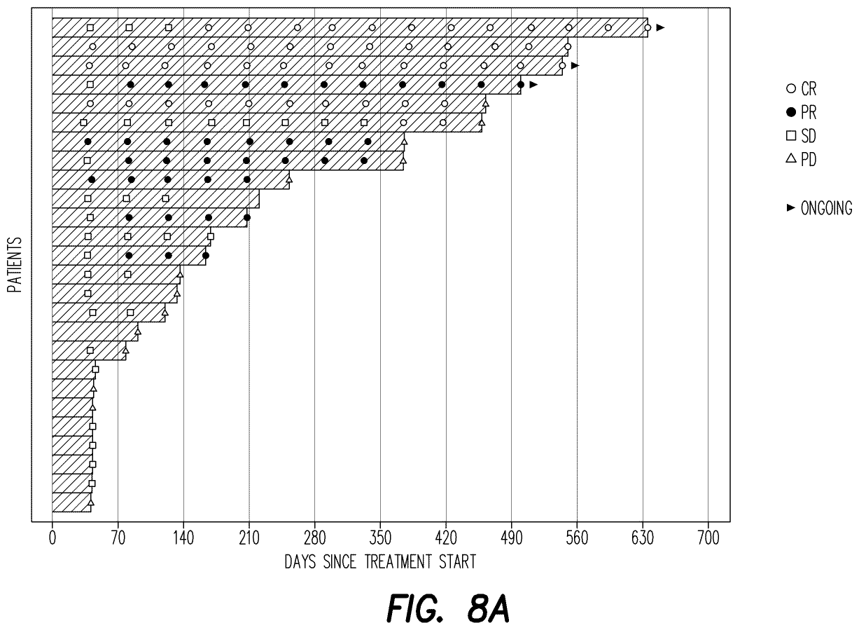

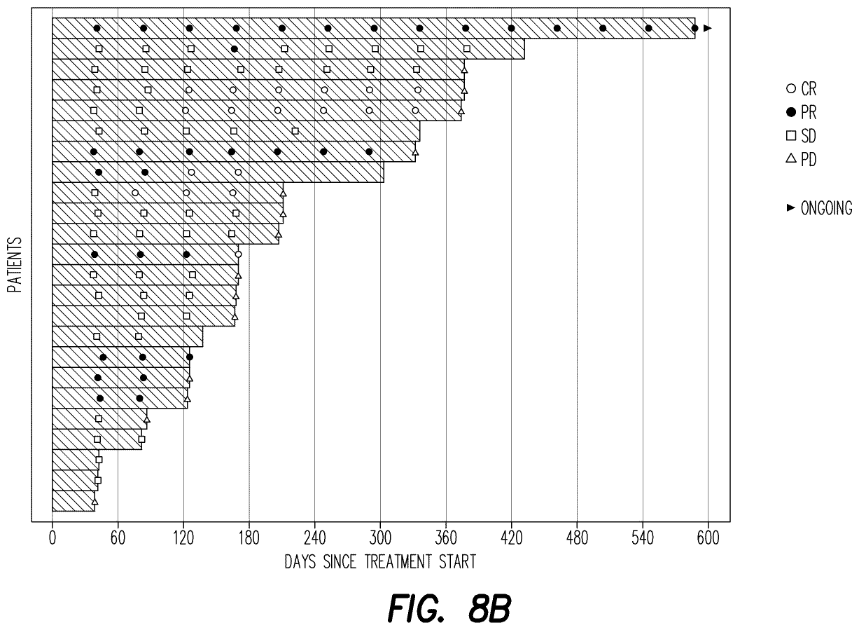

FIG. 8 presents a swimmer plot of intracranial efficacy assessments in cohort 1 per RANO-BM (central review assessment) over time.

In addition to RANO-BM, intracranial response in cohort 1 was assessed according to CNS RECIST 1.1, i.e., a RECIST 1.1 assessment restricted to CNS lesions, by central independent review for patients with measurable CNS lesions at baseline. Results are presented in Table 17. Zongertinib demonstrated promising intracranial responses by RECIST v1.1 in the patients with stable, asymptomatic brain metastases of Cohort 1. In total, 8 patients at 120 mg zongertinib and 9 patients at 240 mg zongertinib had RECIST 1.1 measurable CNS lesions at baseline. Among the 8 patients at 120 mg, 4 showed a partial response and 4 stable disease, leading to an intracranial ORR of 50% and DCR of 100%. In these patients, the median DoR was 12.5 months (95% CI 11.1-NC) and the median follow-up for DoR was 16.6 months (95% CI 11.0-16.6). Among the 9 patients at 240 mg, 1 showed a completed response, 3 partial response and 5 stable disease, so that the intracranial ORR was 44% and DCR 100%.

TABLE 17
Intracranial response to zongertinib 120 mg according
to RECIST 1.1 applied to CNS lesions, as assessed by
blinded independent central review in patients with
measurable CNS lesions at baseline in cohort 1.

| | Cohort 1 | Cohort 1 |
|---|---|---|
| Response Assessment* | 120 mg | 240 mg |
| CNS RECIST 1.1 | (N = 8) | (N = 9) |
| Objective response - no. (%) | 4 (50) | 4 (44) |
| 95% CI | 22-79 | 19-73 |
| Complete response - no. (%) | 0 | 1 (11) |
| Partial response - no. (%) | 4 (50) | 3 (33) |
| Disease control - no. (%) | 8 (100) | 9 (100) |
| 95% CI | 68-100 | 70-100 |
| Stable disease - no. (%) | 4 (50) | 5 (55) |
| Progressive disease - no. (%) | 0 | 0 |
| Not evaluable - no. (%) | 0 | 0 |

*In patients with measurable brain metastases at baseline according to central review

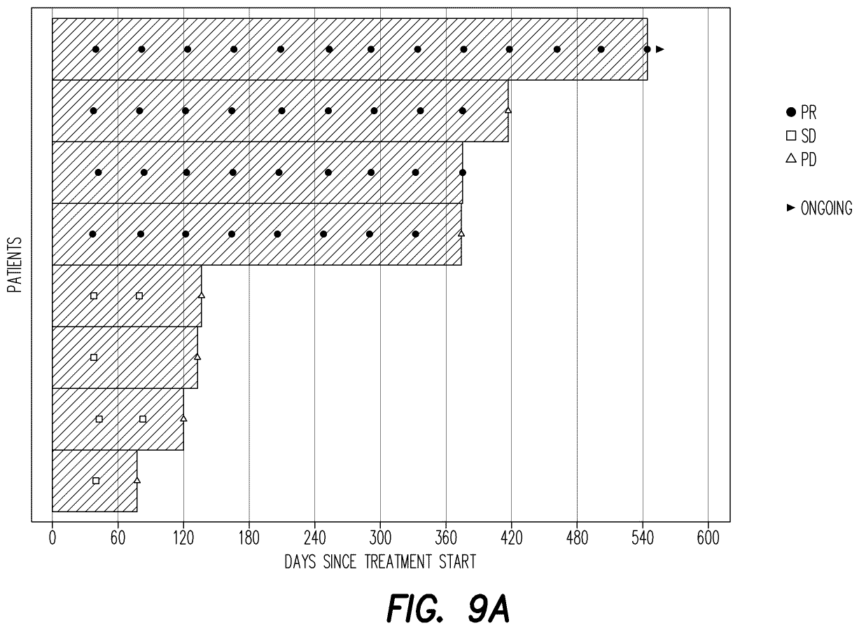

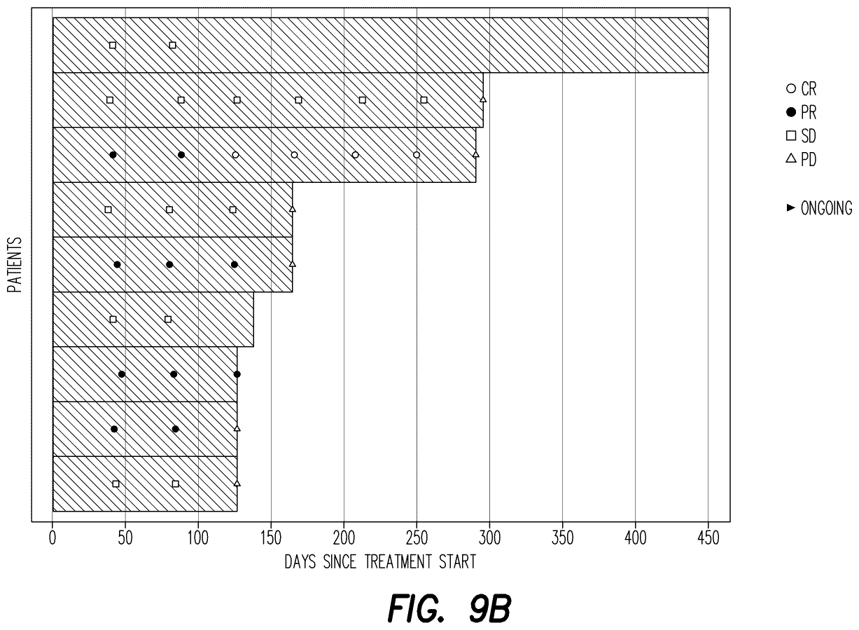

FIG. 9 shows the intracranial efficacy assessments over time in Cohort 1 per RECIST 1.1 applied to CNS lesions (central review) for patients with RECIST 1.1 measurable CNS lesions. Zongertinib demonstrated intracranial activity in patients with brain metastases. The systemic objective response rate according to RECIST, version 1.1, was similar in the subgroup of patients with brain metastases at screening who were treated with zongertinib at a dose of 120 mg (64%) and the overall cohort 1 population treated at the same dose (72%). Additionally, intracranial responses were observed.

### Example 8—Results from Cohort 4 of the Dose Expansion Part (Phase Ib)

This example includes data from cohort 4 of the dose expansion part (Phase Ib) of Example 1 with a data cut-off date of Mar. 26, 2025. Cohort 4 enrolled patients with NSCLC with HER2 mutations in the TKD who had at least one active or symptomatic brain metastasis. Patients were allowed to be either treatment-naïve or pre-treated for NSCLC. In cohort 4, 30 patients were treated at 120 mg QD zongertinib, and 1 patient was treated at 240 mg QD zongertinib. Baseline demographic and disease characteristics data of the patients are presented in Table 18.

TABLE 18
Baseline demographic and disease characteristics of patients
treated with zongertinib 120 mg or 240 mg in cohort 4

| | 120 mg | | 240 mg | |
|---|---|---|---|---|
| Characteristic | (N = 30) | | (N = 1) | |
| Median age - yr (range) | 59 | (38-77) | 62 | (62-62) |

Sex - no. (%)

| | | | | |
|---|---|---|---|---|
| Female | 19 | (63.3) | 1 | (100) |

| | | | |
|---|---|---|---|
| Male | 11 | (36.7) | 0 |

Race - no. (%)

| | | | | |
|---|---|---|---|---|
| Asian | 15 | (50) | 1 | (100) |

| | | | |
|---|---|---|---|
| White | 8 | (26.7) | 0 |
| Black or African American | 2 | (6.7) | 0 |
| Missing^{†} | 5 | (16.7) | 0 |

Eastern Cooperative Oncology Group Performance Status - no. (%)

| 0 | 13 | (43.3) | 0 | |
|---|---|---|---|---|
| 1 | 17 | (56.7) | 1 | (100) |

Tobacco use - no. (%)

| | | | | |
|---|---|---|---|---|
| Never | 19 | (63.3) | 1 | (100) |

| | | | |
|---|---|---|---|
| Former | 10 | (33.3) | 0 |
| Current | 1 | (3.3) | 0 |

Number of previous lines of systemic anticancer treatment - no. (%)

| 0 | 10 | (33.3) | 0 | |
|---|---|---|---|---|
| 1 | 13 | (43.3) | 0 | |
| 2 | 3 | (10.0) | 1 | (100) |

| | | | |
|---|---|---|---|
| 3-4 | 3 | (10.0) | 0 |
| 5-6 | 1 | (3.3) | 0 |

| | | |
|---|---|---|
| ≥7 | 0 | 0 |

Site of metastases at screening - no. (%)

| | | | | |
|---|---|---|---|---|
| Brain | 30 | (100) | 1 | (100) |

| | | | |
|---|---|---|---|
| Liver | 7 | (23.3) | 0 |

Method used for HER2 sequencing^{‡} - no. (%)

| | | | | |
|---|---|---|---|---|
| Next-generation sequencing | 28 | (93.3) | 1 | (100) |

| | | | |
|---|---|---|---|
| Polymerase chain reaction | 1 | (3.3) | 0 |
| Other or unknown | 1 | (3.3) | 0 |

HER2 TKD mutation type - no. (%)

| | | | | |
|---|---|---|---|---|
| A775_G776insYVMA | 17 | (56.7) | 1 | (100) |

| | | | |
|---|---|---|---|
| P780_Y781insGSP | 4 | (13.3) | 0 |
| G776 > VC | 3 | (10.0) | 0 |
| Other | 6 | (20.0) | 0 |

^{†}Due to legal requirements.
^{‡}Local HER2 testing.

The safety profile of zongertinib in cohort 4 is shown in Table 19. Grade 3 or higher drug-related adverse events were reported in 5 patients (17%) in cohort 4. Two patients (6.7%) had adverse events leading to dose reduction of zongertinib. Overall, the safety profile of zongertinib in cohort 4 was comparable to the safety profile observed in the other cohorts, including cohort 1 as discussed e.g. in Examples 3, 4 and 7.

TABLE 19
Safety summary and most common drug-related adverse events in
patients treated with zongertinib 120 mg or 240 mg in cohort 4

| | 120 mg | | | | 240 mg | |
|---|---|---|---|---|---|---|
| | (N = 30) | | | | (N = 1) | |
| | All | | Grade ≥3 | | All | Grade ≥3 |
| Event | no. (%) | | | | | |
| Any treatment-emergent adverse event | 30 | (100) | 19 | (63.3) | 1 (100) | 0 |
| Treatment-emergent adverse event leading | 2 | (6.7) | 2 | (6.7) | 0 | 0 |
| to dose reduction | | | | | | |
| Treatment-emergent adverse event leading | 5 | (16.7) | 4 | (13.3) | 0 | 0 |
| to discontinuation | | | | | | |
| Serious drug-related adverse events | 2 | (6.7) | 2 | (6.7) | 0 | 0 |
| Any drug-related adverse event* | 28 | (93.3) | 5 | (16.7) | 1 (100) | 0 |
| Diarrhoea | 13 | (43.3) | 0 | | 0 | 0 |
| Dry skin | 6 | (20.0) | 0 | | 0 | 0 |
| Pruritus | 6 | (20.0) | 0 | | 0 | 0 |
| Aneamia | 5 | (16.7) | 1 | (3.3) | 0 | 0 |
| Hypokalaemia | 5 | (16.7) | 1 | (3.3) | 0 | 0 |
| Nausea | 5 | (16.7) | 0 | | 0 | 0 |
| Rash | 5 | (16.7) | 0 | | 0 | 0 |
| Alanine aminotransferase increased | 4 | (13.3) | 1 | (3.3) | 0 | 0 |
| Aspartate aminotransferase increased | 3 | (10.0) | 0 | | 0 | 0 |
| Dysgeusia | 3 | (10.0) | 0 | | 0 | 0 |
| Gamma-glutamyltransferase increased | 3 | (10.0) | 1 | (3.3) | 0 | 0 |
| Lipase increased | 3 | (10.0) | 1 | (3.3) | 0 | 0 |

| | | | | | |
|---|---|---|---|---|---|
| Paronychia | 3 | (10.0) | 0 | 0 | 0 |

*As assessed by the investigator; only the events reported in ≥3 (~10%) patients across cohort 4 are shown

The efficacy of zongertinib in cohort 4 was assessed by blinded independent central review using two sets of response criteria:
Intracranial/CNS efficacy per RANO-BM: The primary endpoint in cohort 4 was OR in CNS lesions (defined as a best overall response of PR or CR in CNS lesions, confirmed by a subsequent image) according to the RANO-BM criteria. This set of response criteria only takes into account intracranial response, i.e., response in lesions in the CNS. In addition, DoR, DC and PFS in CNS lesions were analyzed according to RANO-BM.
Systemic efficacy per RECIST 1.1 (including all lesions across the whole body): As a secondary endpoint in cohort 4, the OR according to RECIST 1.1 was assessed by central review. For this assessment, all tumor lesions of the patient are taken into account, including lesions within and outside the CNS. In addition to OR, also DoR, DC and PFS were derived according to RECIST 1.1.

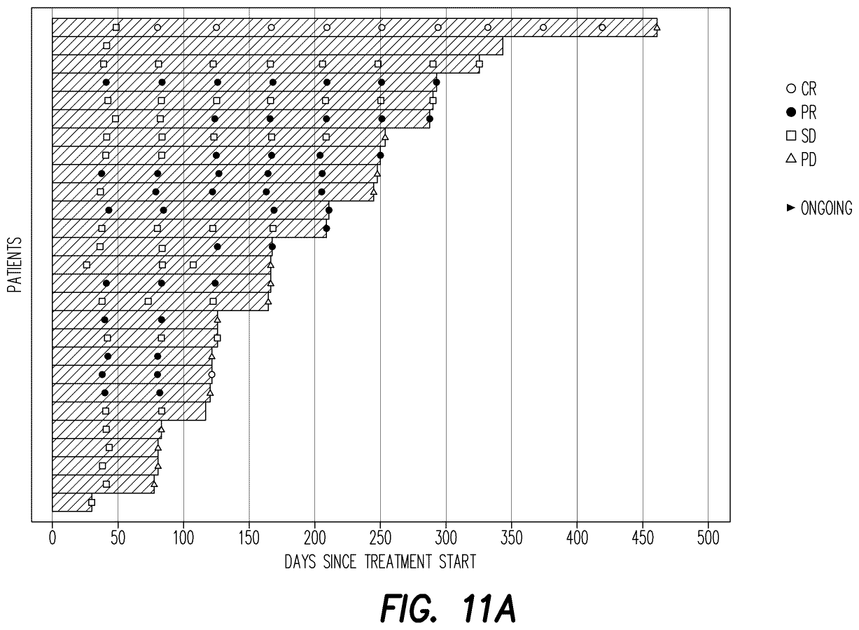

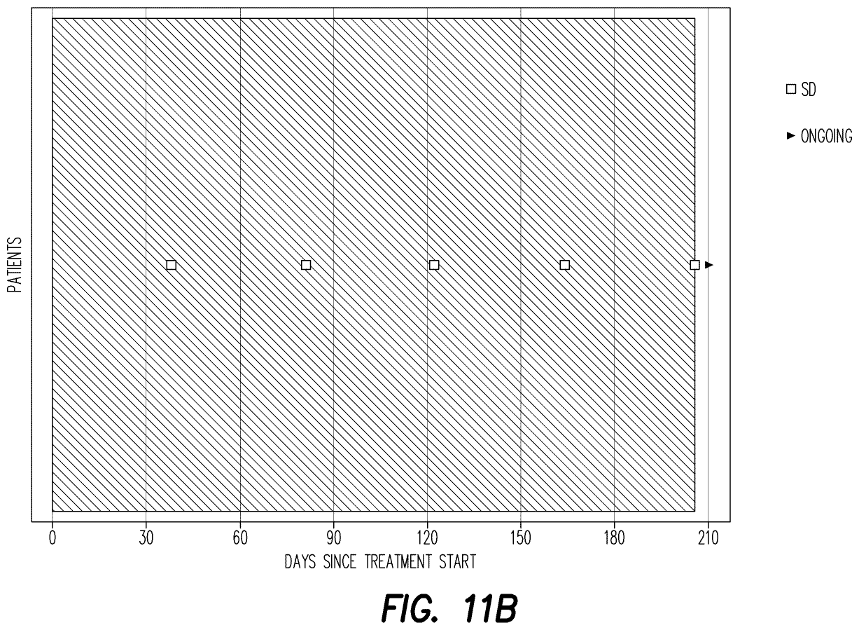

The best overall response in CNS lesions per RANO-BM is shown in Table 20. In addition, OR, DC and DoR in CNS lesions per RANO-BM are presented in Table 21 and PFS in CNS lesions per RANO-BM in Table 22. FIG. 11 presents swimmer plots showing the results of overall response assessments of brain lesions per RANO-BM according to central review over time in all Cohort 4 patients.

Among the 30 patients in cohort 4 treated at 120 mg zongertinib, 13 showed objective response in CNS lesions per RANO-BM, leading to an objective response rate of 43.3%. One of the patients with response achieved a complete response, the remaining 12 responders showed partial response. None of the 13 patients with objective response had received prior radiotherapy for brain lesions before enrolment in the trial.

Median DoR in CNS lesions according to a Kaplan-Meier analysis was 6.9 months, while median PFS in CNS lesions was 8.2 months.

At 240 mg zongertinib, the treated patient achieved a best response of stable disease. The patient is still ongoing at the time of the data cut-off and had so far a PFS in CNS lesions above 6 months.

TABLE 20
Intracranial best overall response according to RANO-BM
as assessed by central review of patients treated
with zongertinib 120 mg or 240 mg in cohort 4.

| | 120 mg | 240 mg |
|---|---|---|
| | (N = 30) | (N = 1) |
| Number of patients - n (%) | 30 (100) | 1 (100) |
| Best overall response | | |
| Complete response - n (%) | 1 (3.3) | 0 |
| Partial response - n (%) | 12 (40.0) | 0 |
| Disease control - no. (%) | 26 (86.6) | 1 (100) |
| Stable disease - n (%) | 13 (43.3) | 1 (100) |
| Progressive disease - n (%) | 0 | 0 |
| Non-evaluable - n (%) | 4 (13.3) | 0 |

TABLE 21
Intracranial objective response, disease control,
and duration of objective response according to RANO-BM
as assessed by central review of patients treated
with zongertinib 120 mg or 240 mg in cohort 4

| | 120 mg | | 240 mg |
|---|---|---|---|
| | (N = 30) | | (N = 1) |
| Number of patients - n (%) | 30 | (100) | 1 (100) |
| Patients with objective response - n (%) | 13 | (43.3) | 0 |
| 95% confidence interval | 27.4-60.8 | | 0-79.3 |
| Patients with disease control - n (%) | 26 | (86.7) | 1 (100) |
| 95% confidence interval | 70.3-94.7 | | 20.7-100 |
| Median duration of objective response | 6.9 | (2.7-12.6) | — |
| from Kaplan-Meier analysis - months | | | |
| (95% CI) | | | |
| Observed number of patients with | 3 | (23.1) | — |
| duration of objective response ≥6 | | | |

months* - n (%)

*calculated relative to the total number of responders

TABLE 22
Intracranial progression-free survival according to
RANO-BM as assessed by central review of patients treated
with zongertinib 120 mg or 240 mg in cohort 4

| | 120 mg | 240 mg |
|---|---|---|
| | (N = 30) | (N = 1) |
| Number of patients - n (%) | 30 (100) | 1 (100) |
| Median progression-free survival | 8.2 | NC* |
| from Kaplan-Meier analysis | | |
| 95% confidence interval | 4.1-15.2 | NC* |
| Patients with progression-free survival | 16 (53.3) | 0 |
| events - n (%) | | |
| Censored patients for progression-free | 14 (46.7) | 1 (100) |
| survival - n (%) | | |
| Ongoing** patients - n (%) | 5 (16.7) | 1 (100) |

*NC means not computable due to number of patients or events
**Ongoing refers to ongoing PFS, i.e., patient is still on treatment and did not experience an event or was censored for a different reason

The systemic best overall response per RECIST 1.1 is shown in Table 23. In addition, systemic OR, DC and DoR per RECIST 1.1 are presented in Table 24 and systemic PFS per RECIST 1.1 in Table 25.

Among the 30 patients in cohort 4 who received 120 mg zongertinib, 19 (63.3%) showed OR per RECIST 1.1, taking into account lesions within and outside of the CNS. Additionally, 9 patients achieved stable disease, leading to a DC rate of 93.3%. The median DoR per RECIST 1.1 was 6.8 months according to a Kaplan-Meier estimate, and the median PFS was also 6.8 months. Four patients (13.3%) still had ongoing PFS at the time of the data cut-off.

The patient treated at 240 mg zongertinib showed a best response of stable disease and achieved a PFS of 10.9 months per RECIST 1.1.

TABLE 23
Best overall response according to RECIST 1.1 (systemic)
as assessed by central review of patients treated
with zongertinib 120 mg or 240 mg in cohort 4

| | 120 mg | 240 mg |
|---|---|---|
| | (N = 30) | (N = 1) |
| Number of patients - n (%) | 30 (100) | 1 (100) |
| Best overall response | | |
| Complete response - n (%) | 0 | 0 |
| Partial response - n (%) | 19 (63.3) | 0 |
| Disease control - no. (%) | 28 (93.3) | 1 (100) |
| Stable disease - n (%) | 9 (30.0) | 1 (100) |
| Progressive disease - n (%) | 1 (3.3) | 0 |
| Non-evaluable - n (%) | 1 (3.3) | 0 |

TABLE 24
Objective response, disease control, and duration
of objective response according to RECIST 1.1 (systemic)
as assessed by central review of patients treated
with zongertinib 120 mg or 240 mg in cohort 4

| | 120 mg | | 240 mg |
|---|---|---|---|
| | (N = 30) | | (N = 1) |
| Number of patients - n (%) | 30 | (100) | 1 (100) |
| Patients with objective response - n (%) | 19 | (63.3) | 0 |
| 95% confidence interval | 45.5-78.1 | | — |
| Patients with disease control - n (%) | 28 | (93.3) | 1 (100) |
| 95% confidence interval | 78.7-98.2 | | 20.7-100 |
| Median duration of objective | 6.8 | (4.0-9.6) | — |
| response from Kaplan-Meier | | | |
| analysis - months (95% CI) | | | |
| Observed number of | 7 | (36.8) | — |
| patients with duration of | | | |
| objective response ≥ 6 | | | |
| months* - n (%) | | | |

*calculated relative to the total number of responders

TABLE 25
Progression-free survival according to RECIST 1.1 (systemic)
as assessed by central review of patients treated with
zongertinib 120 mg or 240 mg in cohort 4

| | 120 mg | 240 mg |
|---|---|---|
| | (N = 30) | (N = 1) |
| Number of patients - n (%) | 30 (100) | 1 (100) |
| Median progression-free survival | 6.8 | 10.9 |
| from Kaplan-Meier analysis | | |
| 95% confidence interval | 4.9-10.1 | NC* |
| Patients with progression-free | 21 (70) | 1 (100) |
| survival events - n (%) | | |
| Censored patients for progression-free | 9 (30) | 0 |
| survival - n (%) | | |
| Ongoing** patients - n (%) | 4 (13.3) | 0 |

*NC means not computable due to number of patients or events
**Ongoing refers to ongoing PFS, i.e., patient is still on treatment and did not experience an event or was censored for a different reason

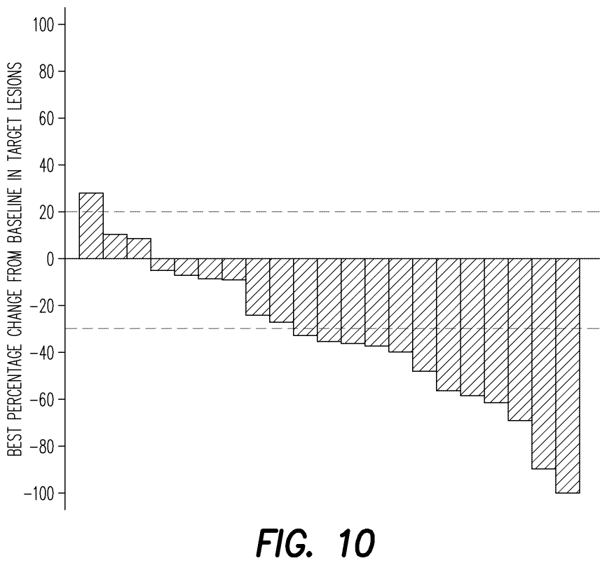

In FIG. 10, the best tumor shrinkage in brain lesions per RANO-BM (investigator assessment) is reported for patients treated at 120 mg in cohort 4. Tumor shrinkage was in median −35% in patients treated at 120 mg zongertinib, with values ranging up to −100% (i.e., disappearance of brain target lesions).

Overall, the data presented in Example 8 indicate that zongertinib is active in patients with symptomatic brain metastases, with similar RECIST 1.1 (all lesions, CNS and non-CNS) and RANO-BM (intracranial) response rates in cohort 4 compared to cohort 1, while also maintaining a manageable safety profile.

### Example 9—Intracranial Efficacy by Prior Brain Radiotherapy in Cohorts 1, 4 and 5 of the Dose Expansion Part (Phase Ib)

In the trial of Example 1, intracranial efficacy was additionally analyzed based on whether the patient had received prior brain radiotherapy. For this analysis, patients were assigned to the following 3 groups:
Patients who did not receive prior radiotherapy for brain lesions;
Patients who received prior radiotherapy for brain lesions less than 60 days before treatment start;
Patients who received prior radiotherapy for brain lesions at least 60 days or longer before treatment start.

In each group, the OR in CNS lesions was assessed according to RANO-BM by central independent review. Results of this analysis for cohorts 1, 4, and 5 are reported in Tables 26, 27 and 28, respectively. The data cut-off used for this analysis was Mar. 26, 2025.

In cohort 1 for patients treated with 120 mg or 240 mg, activity on brain lesions was observed independently of prior brain radiotherapy.

In cohort 4, all responders per RANO-BM had not received any prior brain radiotherapy, indicating that the intracranial responses of patients in cohort 4 can be attributed to the treatment with zongertinib.

Overall, the data of Example 9 indicate that zongertinib is efficacious both for patients who had received prior brain radiotherapy and for patients who had not received it.

TABLE 26
Efficacy in CNS lesions according to RANO-BM per central review by prior brain radiotherapy (RT) grouping
of patients with brain metastases at baseline treated with zongertinib 120 mg or 240 mg in cohort 1

| | Subgroup | | | | | | | | | | | |
|---|---|---|---|---|---|---|---|---|---|---|---|---|
| | Brain RT < 60 days | | | | Brain RT ≥ 60 days | | | | No prior brain RT | | | |
| Objective and best overall | before treatment | | | | before treatment | | | | | | | |
| response | 120 mg | | 240 mg | | 120 mg | | 240 mg | | 120 mg | | 240 mg | |
| Number of patients - n (%) | 6 | (100) | 2 | (100) | 5 | (100) | 9 | (100) | 17 | (100) | 14 | (100) |
| Patients with objective | 5 | (83.3) | 1 | (50) | 1 | (20) | 1 | (11.1) | 5 | (29.4) | 8 | (57.1) |
| response - n (%) | | | | | | | | | | | | |
| 95% confidence | 43.6-97.0 | | 9.5-90.5 | | 3.6-62.4 | | 2.0-43.5 | | 13.3-53.1 | | 32.6-78.6 | |
| interval | | | | | | | | | | | | |
| Complete response | 2 | (33.3) | 0 | | 0 | | 0 | | 2 | (11.8) | 4 | (28.6) |
| Partial response | 3 | (50.0) | 1 | (50) | 1 | (20) | 1 | (11.1) | 3 | (17.6) | 4 | (28.6) |
| Patients with disease control - | 6 | (100) | 2 | (100) | 3 | (60) | 8 | (88.9) | 13 | (76.5) | 13 | (92.9) |
| n (%) | | | | | | | | | | | | |
| Stable disease - n (%) | 1 | (16.7) | 1 | (50) | 2 | (40) | 7 | (77.8) | 8 | (47.1) | 5 | (35.7) |

| | | | | | | | | | |
|---|---|---|---|---|---|---|---|---|---|
| Progressive disease - n (%) | 0 | 0 | 2 | (40) | 1 | (11.1) | 2 | (11.8) | 0 |

| | | | | | | | | |
|---|---|---|---|---|---|---|---|---|
| Non evaluable - n (%) | 0 | 0 | 0 | 0 | 2 | (11.8) | 1 | (7.1) |

TABLE 27
Efficacy in CNS lesions according to RANO-BM per central review by prior brain radiotherapy
(RT) grouping of patients treated with zongertinib 120 mg or 240 mg in cohort 4

| | Subgroup | | | | | | | | | |
|---|---|---|---|---|---|---|---|---|---|---|
| | Brain RT < 60 days | | | Brain RT ≥ 60 days | | | No prior brain RT | | | |
| Objective and best overall | before treatment | | | before treatment | | | | | | |
| response | 120 mg | | 240 mg | 120 mg | | 240 mg | 120 mg | | 240 mg | |
| Number of patients - n (%) | 1 | (100) | 0 | 5 | (100) | 0 | 24 | (100) | 1 | (100) |
| Patients with objective | 0 | | — | 0 | | — | 13 | (54.2) | 0 | |
| response - n (%) | | | | | | | | | | |
| 95% confidence | 0-79.3 | | — | 0-43.4 | | — | 35.1-72.1 | | 0-79.3 | |
| interval | | | | | | | | | | |
| Complete response | 0 | | — | 0 | | — | 1 | (4.2) | 0 | |
| Partial response | 0 | | — | 0 | | — | 12 | (50.0) | 0 | |
| Disease control - n (%) | 1 | (100) | — | 4 | (80) | — | 21 | (87.5) | 1 | (100) |
| Stable disease - n (%) | 1 | (100) | — | 4 | (80) | — | 8 | (33.3) | 1 | (100) |

| Progressive disease - n (%) | 0 | — | 0 | | — | 0 | | 0 |
|---|---|---|---|---|---|---|---|---|
| Non evaluable - n (%) | 0 | — | 1 | (20) | — | 3 | (12.5) | 0 |

TABLE 28
Efficacy in CNS lesions according to RANO-BM per central review by prior
brain radiotherapy (RT) grouping of patients with brain metastases
at baseline treated with zongertinib 120 mg or 240 mg in cohort 5

| | Subgroup | | | | | | | | | | |
|---|---|---|---|---|---|---|---|---|---|---|---|
| | Brain RT < 60 days | | | Brain RT ≥ 60 days | | | | No prior brain RT | | | |
| Objective and best overall | before treatment | | | before treatment | | | | | | | |
| response | 120 mg | | 240 mg | 120 mg | | 240 mg | | 120 mg | | 240 mg | |
| Number of patients - n (%) | 4 | (100) | 0 | 6 | (100) | 2 | (100) | 13 | (100) | 1 | (100) |

| Patients with objective | 1 | (25) | — | 0 | 0 | 3 | (23.1) | 1 | (100) |
|---|---|---|---|---|---|---|---|---|---|
| response - n (%) | | | | | | | | | |
| 95% confidence | 4.6-69.9 | | — | 0-39.0 | 0-65.8 | 8.2-50.3 | | 20.7-100 | |
| interval | | | | | | | | | |
| Complete response | 0 | | — | 0 | 0 | 1 | (7.7) | 0 | |
| Partial response | 1 | (25) | — | 0 | 0 | 2 | (15.4) | 1 | (100) |

| | | | | | | | | | | |
|---|---|---|---|---|---|---|---|---|---|---|
| Disease control - n % | 4 | (100) | — | 4 | (66.7) | 1 | (50) | 9 | (69.2) | 0 |
| Stable disease - n (%) | 3 | (75) | — | 4 | (66.7) | 1 | (50) | 6 | (46.2) | 0 |

| | | | | | | | | | |
|---|---|---|---|---|---|---|---|---|---|
| Progressive disease - n (%) | 0 | — | 1 | (16.7) | 1 | (50) | 1 | (7.7) | 0 |

| | | | | | | | | |
|---|---|---|---|---|---|---|---|---|
| Non evaluable - n (%) | 0 | — | 1 | (16.7) | 0 | 3 | (23.1) | 0 |

### Example 10—Site of Disease Progression in Cohorts 1, 4 and 5 of the Dose Expansion Part (Phase Ib)

The site of disease progression in cohort 1, 4 and 5 patients who progressed following treatment with zongertinib 120 mg or 240 mg was assessed by the investigator in the trial of Example 1. For this analysis, the investigator performed radiological response assessments per three separate sets of criteria:
Systemic RECIST 1.1 (including lesions within and outside the CNS);
Non-CNS RECIST 1.1 (RECIST 1.1 assessment restricted to lesions outside of the CNS);
RANO-BM (taking into account only lesions within the CNS).

For patients who showed a radiological assessment of progressive disease according to systemic RECIST 1.1, the above assessments allow to assign a site of progression to this event. Progressions were classified in one of four categories: (1) isolated CNS progression (RANO-BM progression without non-CNS RECIST 1.1 progression), (2) isolated non-CNS progression (non-CNS RECIST 1.1 progression without RANO-BM progression), (3) simultaneous CNS/non-CNS progression (both non-CNS and CNS progression assessed at the same visit), and (4) unknown/unreported site of progression (all other cases, including patients where a RECIST 1.1 progression without RANO-BM or non-CNS RECIST 1.1 progression occurred).

The results of this analysis for cohorts 1, 4, and 5 are reported in Tables 29, 30, and 31, respectively. The data cut-off used for this analysis was Mar. 26, 2025. In 63% of cohort 1 patients who progressed when taking zongertinib treatment at 120 mg, the progression occurred outside of the CNS. The major site of progression was similar in cohort 1 patients treated with 240 mg zongertinib. In cohort 4, a higher rate of isolated CNS progressions was observed (50%), while the site of progression in cohort 5 differed depending on the zongertinib dose.

TABLE 29
Site of progression in patients treated with zongertinib
120 mg or 240 mg in cohort 1 who progressed according
to RECIST 1.1 (investigator assessment)

| | 120 mg QD | | 240 mg QD | |
|---|---|---|---|---|
| | N | % | N | % |
| Number of patients | 75 | — | 57 | — |
| Patients with progression | 46 | 100 | 43 | 100 |
| events (RECIST 1.1) | | | | |
| Isolated CNS progression | 7 | 15.2 | 11 | 25.6 |
| Isolated non-CNS progression | 29 | 63.0 | 24 | 55.8 |
| Simultaneous CNS/non-CNS progression | 5 | 10.9 | 2 | 4.7 |
| Unknown site of progression | 5 | 10.9 | 6 | 14.0 |

TABLE 30
Site of progression in patients treated with zongertinib
120 mg or 240 mg in cohort 4 who progressed according
to RECIST 1.1 (investigator assessment)

| | 120 mg QD | | 240 mg QD | |
|---|---|---|---|---|
| | N | % | N | % |
| Number of patients | 30 | — | 1 | — |
| Patients with progression | 20 | 100 | 0 | — |
| events (RECIST 1.1) | | | | |
| Isolated CNS progression | 10 | 50.0 | — | — |
| Isolated non-CNS progression | 4 | 20.0 | — | — |
| Simultaneous CNS/non-CNS progression | 4 | 20.0 | — | — |
| Unknown site of progression | 2 | 10.0 | — | — |

TABLE 31
Site of progression in patients treated with zongertinib
120 mg or 240 mg in cohort 5 who progressed according
to RECIST 1.1 (investigator assessment)

| | 120 mg QD | | 240 mg QD | |
|---|---|---|---|---|
| | N | % | N | % |
| Number of patients | 31 | — | 8 | — |
| Patients with progression | 22 | 100 | 4 | 100 |
| events (RECIST 1.1) | | | | |
| Isolated CNS progression | 11 | 50.0 | 1 | 25.0 |
| Isolated non-CNS progression | 7 | 31.8 | 2 | 50.0 |
| Simultaneous CNS/non-CNS progression | 3 | 13.6 | 0 | 0 |
| Unknown site of progression | 1 | 4.5 | 1 | 25.0 |

For patients in cohorts 1 and 5, the analysis of the site of progression was also performed restricted to patients who had CNS lesions at baseline. For cohort 4, this analysis was not done since all patients had CNS metastases at baseline as required by the inclusion criteria. The results of this analysis are shown in Tables 32 and 33.

TABLE 32
Site of progression in patients with CNS lesions at baseline
treated with zongertinib 120 mg or 240 mg in cohort 1 who progressed
according to RECIST 1.1 (investigator assessment)

| | 120 mg QD | | 240 mg QD | |
|---|---|---|---|---|
| | N | % | N | % |
| Number of patients | 28 | — | 25 | — |
| Patients with progression | 22 | 100 | 20 | 100 |
| events (RECIST 1.1) | | | | |
| Isolated CNS progression | 7 | 31.8 | 11 | 55.0 |
| Isolated non-CNS progression | 7 | 31.8 | 6 | 30.0 |
| Simultaneous CNS/non-CNS progression | 5 | 22.7 | 2 | 10.0 |
| Unknown site of progression | 3 | 13.6 | 1 | 5.0 |

TABLE 33
Site of progression in patients with CNS lesions at baseline
treated with zongertinib 120 mg or 240 mg in cohort 5 who progressed
according to RECIST 1.1 (investigator assessment)

| | 120 mg QD | | 240 mg QD | |
|---|---|---|---|---|
| | N | % | N | % |
| Number of patients | 23 | — | 3 | — |
| Patients with progression | 18 | 100 | 2 | 100 |
| events (RECIST 1.1) | | | | |
| Isolated CNS progression | 11 | 61 | 1 | 50 |
| Isolated non-CNS progression | 4 | 22 | 0 | 0 |
| Simultaneous CNS/non-CNS progression | 3 | 17 | 0 | 0 |
| Unknown site of progression | 0 | 0 | 1 | 50 |

### Example 11—Pooled Results from Cohorts 1 and 4 of the Dose Expansion Part (Phase Ib)

Given that the populations of Cohorts 1 and 4 are comparable in terms of prior therapies and baseline mutations, it was decided to perform a pooled analysis of efficacy in Cohort 1 and Cohort 4 patients with brain metastases at baseline to allow for inferences on intracranial efficacy with larger sample size. For this, three analysis sets were considered:
Brain metastases analysis set: any Cohort 1 or Cohort 4 patients with brain metastases at baseline
Brain metastases analysis set without prior brain radiotherapy: any patient in the brain metastases analysis set who did not have any radiotherapy targeting CNS lesions before starting zongertinib (regardless of timeframe)
Brain metastases set with RECIST 1.1 measurable brain lesions: any patient in the brain metastases analysis set who had RECIST 1.1 measurable lesions in the brain as assessed by central review

As of the data cut-off date of this Example, Mar. 26, 2025, 75 patients in Cohort 1 and 30 patients in Cohort 4 had received zongertinib 120 mg QD.

Pooled baseline characteristics are summarised in Table 34.

TABLE 34
Pooled baseline characteristics of patients
enrolled in cohort 1 and 4 of Phase Ib

| | | | Stable, | | | |
|---|---|---|---|---|---|---|
| | | | asymptomatic | | | |
| | No brain | | brain | | Active brain | |
| | metastases | | metastases | | metastases | |
| | at baseline | | at baseline | | at baseline | |
| | (N = 47) | | (N = 28) | | (N = 30) | |
| Median age, | 60 | (30-80) | 63 | (32-80) | 59 | (38-77) |
| years (range) | | | | | | |
| Female, n (%) | 32 | (68) | 19 | (68) | 19 | (63) |

Race, n (%) ^{†}

| | | | | | | |
|---|---|---|---|---|---|---|
| Asian | 26 | (55) | 14 | (50) | 15 | (50) |
| Non-Asian | 20 | (43) | 9 | (32) | 10 | (33) |

Lines of prior systemic anticancer treatment, n (%)

| 0 lines | 2 | (4)* | 1 | (4)* | 10 | (33) |
|---|---|---|---|---|---|---|
| 1 line | 32 | (68) | 11 | (39) | 13 | (43) |
| ≥2 lines | 13 | (28) | 16 | (57) | 7 | (23) |
| No prior brain | NA | | 17 | (60) | 24 | (80) |
| radiotherapy | | | | | | |
| ECPG PS, n (%) | | | | | | |
| 0 | 16 | (34) | 12 | (43) | 13 | (43) |
| 1 | 31 | (66) | 16 | (57) | 17 | (57) |

^{†} Missing: n = 1 in Cohort 1 no brain metastases group and n = 5 in both Cohort 1 and 4 brain metastases groups.
*Patients had received previous treatment in an adjuvant context only but within 6 months before initiating zongertinib.

The manageable safety profile of zongertinib was consistent in patients with and without brain metastases. Intracranial efficacy per RANO-BM is reported in Tables 35 and 36 for the brain metastases analysis set, Tables 37 and 38 for the set without prior brain radiotherapy, and in Tables 39 and 40 for the set with measurable brain lesions.

In total 58 patients were included in the brain metastases analysis set at 120 mg QD. Among those, 24 showed objective response in the brain according to RANO-BM by central review (ORR: 41%, 95% CI: 30-54%), with 5 patients achieving complete responses (9%) and 19 patients partial responses (33%). The disease control rate was 83% (48 patients). Further, a median intracranial DoR of 11.1 months (95% CI: 5.5—not calculable) and a median intracranial PFS of 8.2 months (95% CI: 4.5-12.3) were seen. In these patients, the confirmed site of disease progression was: 29% isolated CNS progression, 19% isolated non-CNS progression, 16% simultaneous CNS/non-CNS progression and 9% unknown, where the remaining patients did not show a progression event.

In the two subsets of the brain metastases analysis set, consistent results to the overall population were observed: among the 41 patients with brain metastases at 120 mg who did not receive prior brain radiotherapy, the intracranial ORR was 44% (18 patients, 95% CI: 30-59) and the DCR was 83% (34 patients), with a median intracranial DoR of 6.9 months (95% CI: 2.9-12.6) and median intracranial PFS of 8.1 months (95% CI 4.1-12.3).

At 240 mg zongertinib, the brain metastases analysis set included 26 patients, with an intracranial ORR of 39% (10 patients) according to RANO-BM per central review, where 15% (4 patients) had a complete response. Median intracranial PFS was 6.9 months, and median intracranial DoR was 8.3 months. Overall, 240 mg and 120 mg led to similar intracranial efficacy in the brain metastases analysis set and the two considered subsets.

TABLE 35
Intracranial efficacy in terms of response per RANO-BM (central
review) for patients in the brain metastases analysis set

| | |
|---|---|
| 120 mg QD | 240 mg QD |

| | N | % | N | % |
|---|---|---|---|---|
| Number of patients | 58 | 100 | 26 | 100 |
| Patients with | 24 | 41 | 10 | 39 |
| objective response | | | | |

| | | |
|---|---|---|
| 95% confidence interval | 30-54 | 22-58 |

| | | | | |
|---|---|---|---|---|
| Complete response | 5 | 9 | 4 | 15 |
| Partial response | 19 | 33 | 6 | 23 |
| Patients with disease control | 48 | 83 | 24 | 92 |

| | | |
|---|---|---|
| 95% confidence interval | 71-90 | 76-98 |

| | | | | |
|---|---|---|---|---|
| Stable disease | 24 | 41 | 14 | 54 |
| Progressive disease | 4 | 7 | 1 | 4 |
| Not evaluable | 6 | 10 | 1 | 4 |

TABLE 36
Intracranial efficacy in terms of duration of response
and progression free-survical per RANO-BM (central review)
for patients in the brain metastases analysis set

| | 120 mg QD | | 240 mg QD | |
|---|---|---|---|---|
| Number of patients | 58 | | 26 | |
| Median progression-free survival in CNS | 8.2 | | 6.9 | |
| 95% confidence interval (months) | 4.5-12.3 | | 5.5-11.0 | |
| Patients with events - N (%) | 31 | (53) | 18 | (69) |
| Patients with ongoing PFS - N (%) | 8 | (14) | 2 | (8) |
| Number of patients with objective response | 24 | | 10 | |
| Median duration of response (months) | 11.1 | | 8.3 | |
| 95% confidence interval (months) | 5.5-NC | | 2.7-9.7 | |
| Patients with events - N (%*) | 12 | (50) | 7 | (70) |
| Patients with ongoing DoR - N (%*) | 5 | (21) | 1 | (10) |

| Median follow-up for DoR (months) | 13.7 |
|---|---|
| 95% confidence interval (months) | 5.4-16.6 |

NC: non calculable due to number of events/censored patients
*For duration of response, the percentage of patients ongoing and patients with events are calculated relative to the number of responders.

TABLE 37
Intracranial efficacy in terms of response per RANO-BM
(central review) for patients in the brain metastases
analysis set who did not receive prior brain radiotherapy

| | 120 mg QD | | 240 mg QD | |
|---|---|---|---|---|
| | N | % | N | % |
| Number of patients | 41 | 100 | 15 | 100 |
| Patients with objective response | 18 | 44 | 8 | 53 |
| 95% confidence interval | 30-59 | | 30-75 | |
| Complete response | 3 | 7 | 4 | 27 |
| Partial response | 15 | 37 | 4 | 27 |
| Patients with disease control | 34 | 83 | 14 | 93 |
| 95% confidence interval | 69-92 | | 70-99 | |
| Stable disease | 16 | 39 | 6 | 40 |
| Progressive disease | 2 | 5 | 0 | 0 |
| Not evaluable | 5 | 12 | 1 | 7 |

TABLE 38
Intracranial efficacy in terms of duration of response
and progression free-survical per RANO-BM (central
review) for patients in the brain metastases analysis
set who did not receive prior brain radiotherapy

| | 120 mg QD | | 240 mg QD | |
|---|---|---|---|---|
| Number of patients | 41 | | 15 | |
| Median progression-free survival in CNS | 8.1 | | 6.9 | |
| 95% confidence interval (months) | 4.1-12.3 | | 4.5-11.0 | |
| Patients with events - N (%) | 22 | (54) | 11 | (73) |
| Patients with ongoing PFS - N (%) | 5 | (12) | 1 | (7) |
| Number of patients with objective response | 18 | | 8 | |
| Median duration of response (months) | 6.9 | | 8.3 | |
| 95% confidence interval (months) | 2.9-12.6 | | 2.7-8.6 | |
| Patients with events - N (%*) | 10 | (56) | 7 | (88) |

| | | | |
|---|---|---|---|
| Patients with ongoing DOR - N (%*) | 3 | (17) | 0 |

NC: non calculable due to number of events/censored patients
*For duration of response, the percentage of patients ongoing and patients with events are calculated relative to the number of responders.

TABLE 39
Intracranial efficacy in terms of response per RANO-BM (central
review) for patients in the brain metastases analysis set with
RECIST 1.1 measurable CNS lesions at baseline percentral review

| | 120 mg QD | | 240 mg QD | |
|---|---|---|---|---|
| | N | % | N | % |
| Number of patients | 29 | 100 | 10 | 100 |
| Patients with objective response | 15 | 52 | 4 | 40 |
| 95% confidence interval | 34-69 | | 17-69 | |
| Complete response | 1 | 3 | 1 | 10 |
| Partial response | 14 | 48 | 3 | 30 |
| Patients with disease control | 28 | 97 | 9 | 90 |
| 95% confidence interval | 83-99 | | 60-98 | |
| Stable disease | 13 | 45 | 5 | 50 |
| Progressive disease | 0 | 0 | 1 | 10 |
| Not evaluable | 1 | 3 | 0 | 0 |

TABLE 40
Intracranial efficacy in terms of duration of response and
progression free-survical per RANO-BM (central review) for
patients in the brain metastases analysis set with RECIST
1.1 measurable CNS lesions at baseline percentral review

| | 120 mg QD | | 240 mg QD | |
|---|---|---|---|---|
| Number of patients | 29 | | 10 | |
| Median progression-free survival in CNS | 8.1 | | 5.5 | |
| 95% confidence interval (months) | 4.4-11.3 | | 1.3-12.4 | |
| Patients with events - N (%) | 21 | (72) | 7 | (70) |
| Patients with ongoing PFS - N (%) | 3 | (10) | 1 | (10) |
| Number of patients with objective response | 15 | | 4 | |
| Median duration of response (months) | 6.9 | | 2.8 | |
| 95% confidence interval (months) | 2.9-11.1 | | 2.7-8.3 | |
| Patients with events - N (%*) | 10 | (67) | 3 | (75) |

| | | | |
|---|---|---|---|
| Patients with ongoing DOR - N (%*) | 1 | (7) | 0 |

NC: non calculable due to number of events/censored patients
*For duration of response, the percentage of patients ongoing and patients with events are calculated relative to the number of responders.

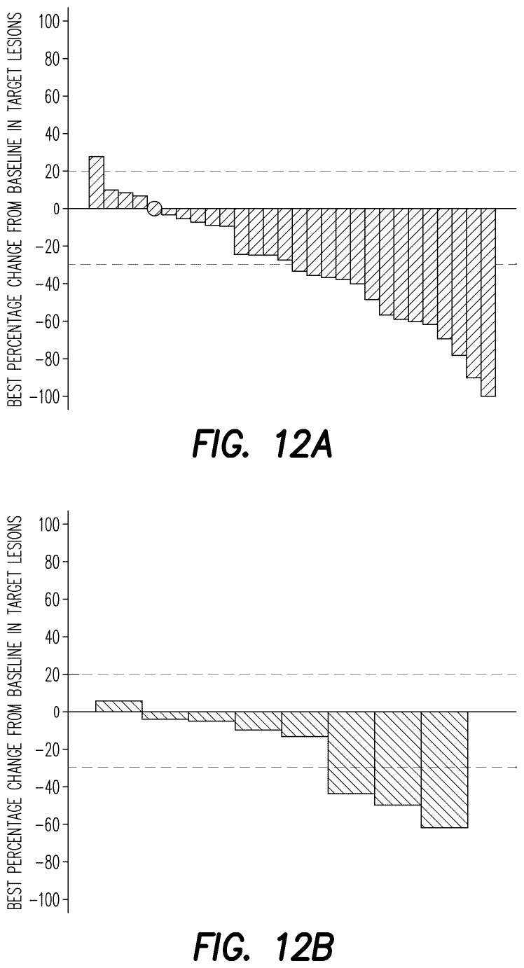

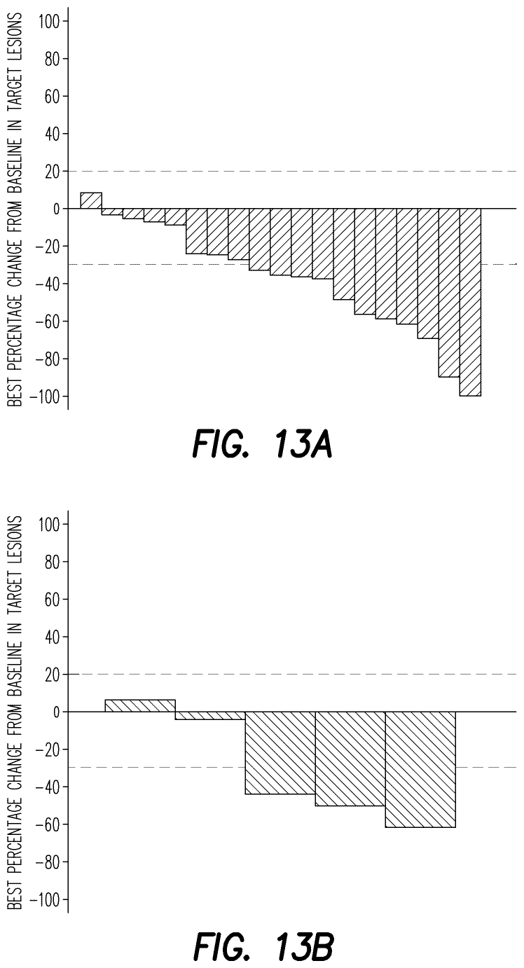

Best tumor shrinkage in brain lesion in all patients of the brain metastases set as assessed by the investigator according to RANO-BM is shown in FIG. 12. Additionally, tumor shrinkage in the set of patients without prior radiotherapy is shown in FIG. 13.

28 of the 58 patients treated at 120 mg in the complete brain metastases analysis set had target lesions per RANO-BM according to the investigator. The best tumor shrinkage in this group was in median −30.2% (mean: −31.7%), with values ranging up to −100%, where the latter indicates a complete disappearance of target lesions. Among the 28 patients with measurements available, 6 (21%) patients showed a reduction in lesion size between-30% and −50% from baseline, and 8 (29%) patients showed a reduction in lesion size of more than-50%.

In the set of patients at 120 mg who did not receive prior brain lesion radiotherapy, 19 patients had brain lesion measurements available according to the investigator assessment. Notably, 95% (18 patients) showed a decrease in brain lesion size from baseline, and 58% (11 patients) showed a decrease of at least-30% in brain lesion size in this group. Among the 19 patients without prior brain radiotherapy, 5 had not received any systemic prior anti-cancer therapies in a metastatic/advanced setting for NSCLC (treatment naïve). In the 5 treatment-naïve patients, median tumor shrinkage was −56%, with a range of −33% to −100%. Among the 14 patients who had received systemic prior anti-cancer therapy (but no prior radiotherapies for brain metastases), the median tumor shrinkage was −26%, with shrinkage up to −90% observed. Within these 14 patients, 43% (6 patients) had tumor shrinkage of at least-30%.

In the 240 mg dose group, 8 patients in the brain metastases analysis set had brain tumor measurements available per RANO-BM according to the investigator. The median tumor shrinkage among these 8 patients was −11.7% (mean: −22.8%), where tumor shrinkage up to −62% was observed in target brain lesions.

Overall, the results of the pooled analysis of intracranial efficacy show that zongertinib leads to clinical benefit for patients with brain lesions in an analysis set with larger sample size, which emphasizes the consistent efficacy of zongertinib across different types of patients with brain metastases at baseline. In particular, comparable efficacy was observed for patients with both active and stable brain metastases at baseline, and also for patients with and without prior radiotherapy for brain lesions before start of zongertinib.

### Example 12—Protocol of a Phase II, Multicentre, Multicohort, Open-Label Trial to Evaluate the Efficacy and Safety of Oral Zongertinib for the Treatment of Selected HER2-Mutated or Overexpressed/Amplified Solid Tumours

In the present Example, a clinical trial is performed to test zongertinib monotherapy in a variety of HER2 aberrant cancers. Patients with stable brain metastases are eligible for this trial.

### 12.1 Objectives

The trial assesses the safety and efficacy of zongertinib monotherapy in patients with histologically or cytologically confirmed advanced and unresectable HER2-mutant or overexpressed/amplified solid tumours.

The primary objective of the trial is to assess the anti-tumour activity of zongertinib monotherapy in a variety of solid tumours.

The secondary objectives are to evaluate the efficacy, safety, tolerability, and risk-benefit profile of zongertinib and evaluate the PROs.

### 12.2 Endpoints

The primary endpoint applies to each of the cohorts. The primary endpoint is OR, defined as the best overall response of confirmed CR or confirmed PR according to RECIST 1.1, as assessed by central independent review, from the date of treatment start until the earliest date of PD, death, or last evaluable tumour assessment before the start of subsequent anti-cancer therapy, or trial treatment discontinuation.

The primary measure of interest is the proportion of patients with OR according to RECIST 1.1 by central assessment, and it is summarised as the median of the respective marginal posterior response rate derived with the Bayesian hierarchical model ExNex Adjusted.

The summary measure of OR includes all treated patients regardless of breaks from trial treatment but excludes the effects of any subsequent anti-cancer therapy started before progression.

All secondary endpoints apply to each of the cohorts. The secondary endpoints include:
DoR, defined as the time from first documented OR according to RECIST 1.1 until the earliest date of disease progression or death among patients with confirmed objective response, assessed by central independent review;
PFS, defined as the time from treatment start until the earliest date of tumour progression according to RECIST 1.1 assessed by central independent review, or death from any cause, whichever occurs first;
DC, defined as best overall response of CR or PR or SD where best overall response is defined according to RECIST 1.1 from first treatment administration until the earliest of disease progression, death, or last evaluable tumour assessment before the start of subsequent anti-cancer therapy, or treatment discontinuation, as assessed by central independent review;
Overall survival (OS), defined as the time from start of treatment to death from any cause;
Occurrence of treatment-emergent AEs;
Change from baseline to Week 48 or PD, if earlier, in the European Organisation for Research and Treatment of Cancer quality of life questionnaire (EORTC QLQ-C30).

Further endpoints applying to each of the cohorts include:
Time to OR according to RECIST 1.1 assessed by central review, defined as the time from first treatment administration until first documented CR or PR;
Tumour shrinkage (in millimeters) as assessed by the investigator is defined as the difference between the minimum post-baseline sum of longest diameters of target lesions (longest for non-nodal lesions, short axis for nodal lesions) and the baseline sum of the longest diameters of the same set of target lesions;
Occurrence of treatment-emergent AEs leading to permanent discontinuation of zongertinib;
Occurrence of treatment-emergent AEs leading to dose reduction of zongertinib;
Occurrence of treatment-emergent AEs of Grade ≥3;
Occurrence of treatment-emergent AEs leading to a treatment interruption;
Occurrence of treatment-emergent hospitalisations;
Duration of treatment-emergent hospitalisations;
Time to permanent discontinuation of zongertinib defined as the time from first administration until the last administration;
PK of zongertinib:
   Cmax: maximum plasma concentration;
   AUC0 t2: Area under the concentration-time curve over time interval from 0 to t2;
Change from baseline to Week 24 or PD by central independent review, whichever is earlier, in PRO-CTCAE (mouth/throat sores, taste changes, nausea, vomiting, diarrhoea, rash, skin dryness, itching, numbness and tingling, nail loss, nail ridging, and nail discoloration);
Change from baseline to Week 48 or PD by central independent review, whichever is earlier, in EORTC IL46 (1 item);
Change from baseline to Week 48 or PD by central independent review, whichever is earlier, in EQ-5D-5L.

### 12.3 Design and Trial Population

This is a Phase II, open-label, multi-centre, multi-cohort trial to evaluate the efficacy and safety of zongertinib for the treatment of selected HER2 mutant or overexpressed/amplified tumours. The trial includes 13 cohorts of different tumour types, each recruiting up to 40 patients. There are 11 tumour-specific cohorts (Cohorts 1 to 5, Cohorts 7 to 9, and Cohorts 11 to 13) and 2 tumour agnostic cohorts (Cohorts 6 and 10). Patients are divided into the following cohorts: Patients with overexpressed/amplified HER2 tumours (with or without HER2 mutation) are enrolled into one of eight cohorts:
Urothelial cancer (Cohort 1);
Biliary tract cancer (cholangiocarcinoma, gallbladder cancer, and ampullary cancer) (Cohort 2);
Uterine cancer (Cohort 3);
Cervical cancer (Cohort 4);
Non-squamous non-small cell lung cancer (NSCLC) (Cohort 5);
Salivary gland cancer (Cohort 11);
Colorectal cancer (Cohort 12);
Any other HER2 overexpressed/amplified solid tumour types (except breast cancer) (Cohort 6).

Patients with mutant HER2 with no or undetermined HER2 overexpression/amplification are enrolled into one of five cohorts:
Urothelial cancer (Cohort 7);
Breast cancer (Cohort 8);
Gastroesophageal adenocarcinoma (GEAC) (Cohort 9);
Biliary tract cancer (cholangiocarcinoma, gallbladder cancer, and ampullary cancer) (Cohort 13);
Any other HER2 mutant solid tumour types (except NSCLC) (Cohort 10).

Additional cohorts may be added via amendment to evaluate other tumour types (e.g. duodenal and small bowel cancer) if >3 patients with a particular tumour type have enrolled in the tumour agnostic cohorts or if efficacy is seen in that tumour type.

### 12.4 Inclusion and Exclusion Criteria

Patients with histologically or cytologically confirmed locally advanced unresectable or metastatic, HER2-mutant or overexpressed/amplified selected solid tumours, who have had at least one prior line of therapy for locally advanced unresectable disease or metastatic disease (adjuvant and neoadjuvant therapy excluded), are eligible for this study.

Main inclusion criteria are:
Patients ≥18 years old or over the legal age of consent in countries where that is greater than 18 years at the time of signature of the ICF.
Documented (previously established by local testing) HER2 status of:
   Cohorts 1 to 6 and 11 to 12: HER2 overexpression/amplification (with or without HER2 mutations), and according to ASCO/CAP gastric cancer guidelines [R23 3591]) defined as:
      HER2 IHC 3+ by gastric algorithm, or
      HER2 IHC 2+ and HER2 amplification by ISH, or
      HER2 amplification in archival tissue by next generation sequencing (NGS) assay, or
      HER2 amplification in ctDNA by blood based NGS assay and investigator confirmation by IHC/ISH prior to or in parallel with the screening process
   Note: In HER2 amplified/overexpressing cohorts patient inclusion per gastric ASCO/CAP guidelines is based on either IHC3+ positive staining, IHC2+ and HER2 amplification by ISH, or a reported HER2 amplification of ≥6 copies by NGS.
   Cohorts 7 to 10 and 13: Known activating HER2 mutations (with no or undetermined HER2 overexpression/amplification) detected in archival tumour tissue or blood by NGS (acceptable mutations are listed in the ISF). Polymerase chain reaction (PCR) is also acceptable, but NGS is preferred.
Availability and willingness to provide a sample of archival formalin-fixed paraffin embedded (FFPE) tumour tissue material taken ≤6 months prior to informed consent. Sample must not be from a lesion irradiated prior to the biopsy and biopsies from brain metastases are not allowed.
Patient with histologically or cytologically confirmed locally advanced unresectable or metastatic solid tumour who has had at least one prior line of therapy for locally advanced unresectable disease or metastatic disease (adjuvant and neoadjuvant therapy excluded). Patient must have documented disease progression or recurrence of disease during or following their latest line of therapy. In the opinion of the Investigator, patients must be unlikely to tolerate or derive clinically meaningful benefit from further standard of care therapy known to prolong survival.
Recovered from any previous therapy-related toxicity to ≤CTCAE Grade 1 at start of treatment (except for alopecia, stable sensory neuropathy, and hypothyroidism [patients on thyroid replacement therapy] which all must be ≤CTCAE Grade 2).
Presence of at least 1 measurable lesion outside the CNS according to RECIST 1.1, as determined by the local site investigator/radiology assessment.
Eastern Cooperative Oncology Group (ECOG) score of 0 or 1.

Main exclusion criteria are:
Diagnosis of HER2 overexpressing/amplified (IHC3+ or IHC2+/ISH+) mBC;
Diagnosis of HER2 mutant NSCLC;
Previous treatment with any HER2 tyrosine kinase inhibitors (TKIs) in the advanced or metastatic setting. In the case of use in the adjuvant setting, at least 12 months must have passed prior to the start of trial drug. Prior ADCs are allowed;
Previous or concomitant malignancies other than the 1 treated in this trial within the previous 3 years except:
   effectively treated non-melanoma skin cancers;
   effectively treated carcinoma in situ of the cervix;
   effectively treated ductal carcinoma in situ of the breast;
   localised prostate cancer on watchful waiting or active surveillance;
   other effectively treated malignancy that is considered cured by local treatment.
Presence of uncontrolled or symptomatic brain or subdural metastases during screening, (unless considered stable by the investigator and local therapy was completed) and patients with known leptomeningeal disease. Use of corticosteroids is allowed if the dose is decreasing or was stable for at least 1 week before screening. Inclusion of patients with newly identified brain metastasis/es at screening is allowed if patients are asymptomatic and stable (as assessed by the investigator) and immediate CNS treatment is unlikely to be required;
Patients who must or wish to continue the intake of restricted medications or any drug considered likely to interfere with the safe conduct of the trial;
Palliative radiotherapy within 2 weeks prior to start of treatment;
Not completely recovered from major surgery (major according to the investigator's assessment) performed prior to screening or planned within 6 months after screening, e.g. hip replacement;
Any history of or concomitant condition that, in the opinion of the investigator, would put the patient at increased risk, compromise the patient's ability to comply with the trial, or interfere with the evaluation of the safety and efficacy of the test drug;
History or presence of cardiovascular abnormalities such as uncontrolled hypertension, congestive heart failure NYHA classification of ≥ III or IV, unstable angina or poorly controlled arrhythmia, which are considered as clinically relevant by the investigator. Myocardial infarction within 28 days of the start of treatment, stroke or pulmonary embolism within 6 months prior to the start of treatment;
Any clinically important abnormalities (as assessed by the investigator) in rhythm, conduction, or morphology of resting ECGs, e.g. complete left bundle branch block, third degree heart block;
Mean resting corrected QT interval (QTcF)>470 msec (females) or >450 msec (males) based on screening 12 lead ECG;
Any factors that increase the risk of QTc prolongation or risk of arrhythmic events such as heart failure, untreated hypokalaemia, congenital long QT syndrome, family history of long QT syndrome or unexplained sudden death under 40 years-of-age;
Ejection fraction <50% or the lower limit of normal of the institutional standard within 28 days prior to the start of treatment.

Additional inclusion and exclusion criteria may apply.

### 12.5 Treatments

Zongertinib is administered orally at 120 mg once daily in 21-day cycles until PD by RECIST 1.1, withdrawal of patient consent, unacceptable toxicity, death, or discontinuation from the trial, whichever occurs first.

### 12.6 Assessments

Tumour assessments should include scans of any known or suspected sites of disease using an appropriate method (CT scan, MRI, PET/CT, or bone scan). As a minimum this should include CT or MRI of the chest, abdomen, and pelvis using i.v. contrast and optional oral contrast if indicated. At screening, a brain scan must be performed to document CNS status (brain MRI is preferred but brain CT will be accepted if MRI is contraindicated). Patients with stable brain metastases at screening should have a brain scan in addition to other required imaging at the same timepoints, using the same modality as screening. Tumour assessments follow RECIST 1.1. The same radiographic procedure must be used throughout the trial. If clinically indicated, imaging of any other known or suspected sites of disease (e.g. bone) using an appropriate method (CT scan, MRI, PET/CT, or bone scan) should be performed.

Assessments are performed by the investigator and sent for central review at the following timepoints (the assessment schedule is independent of treatment cycles and should not be changed even if treatment was interrupted):
Screening (≤28 days prior to initiation of treatment. Note: assessments performed prior to informed consent as part of routine clinical practice are accepted if they meet the requirements of the protocol and are performed within the screening visit window)· Every 2 cycles (6 weeks±5 days) as calculated from C1D1 until PD or start of subsequent anti-cancer treatment. For patients continuing beyond Week 48, imaging schedule will change to every 12 weeks (+1 week) (bone scan every 12 weeks [+1 week] if previously performed).
EOT visit (if not performed within the previous 3 weeks)
For patients without objective progression at the EOT visit, every 12 weeks (+5 days) thereafter until progression, or until one of the following: death, lost to follow up, start of subsequent anti-cancer therapy, withdrawal of consent or until the end of the trial.
Additional scans may also be taken at the discretion of the investigator.

Tumor response by RECIST 1.1 can be evaluated as described in Example 1.

Safety assessments were performed in all treated patients using physical examinations, assessments of vital signs, safety laboratory parameters, electrocardiograms, left ventricular ejection function, and by monitoring the occurrence of adverse events. Adverse events were assessed according to the National Cancer Institute CTCAE version 5.0.

### 12.7 Statistical Methods

The primary analysis is conducted when all cohorts have been completely enrolled, and all patients have been followed for at least 12 weeks or have stopped treatment.

The final analysis is performed once all patients have completed the trial.

For the analysis of primary and secondary endpoints and of those further endpoints that relate to efficacy and safety, all patients in the treated set (TS) (i.e. patients treated with at least 1 dose of trial medication) are included.

For the primary endpoint OR, the proportion of patients with best overall response of complete or partial response is analysed by using an ExNex-Adjusted approach. This is a Bayesian hierarchical model that incorporates a borrowable and a non-borrowable component and target rate adjustments for the ORRs of the cohorts. Next to the median, the mean and 95% credible interval of the respective marginal posterior distribution is used as summary measures of the ORR of each cohort.

An interim analysis is conducted by the sponsor for each cohort except Cohorts 6 and 10. The sponsor may terminate a cohort or the trial for unfavourable results at the interim analysis.

These interim futility analyses is conducted for each cohort at the latest by the time the respective cohort has had 10 patients followed for efficacy for at least 2 scheduled post-baseline tumour assessments, experienced disease progression, died, or permanently discontinued due to any other reason than progression. Recruitment is not be interrupted during the interim futility analyses. No sample size recalculations or adjustments of enrolment targets are foreseen.

### Example 13—a Phase Ib Dose Escalation and Phase II Dose Optimization, Randomized, Open-Label, Multicenter Trial of Oral Zongertinib Alone or in Combination with Other Agents for the Treatment of Patients with Advanced HER2+ Metastatic Breast Cancer (mBC) and Metastatic Gastric, Gastroesophageal Junction, or Esophageal Adenocarcinoma (mGEAC)

In the present Example, a clinical trial is performed to test zongertinib alone or in combination with trastuzumab, with and without capecitabine, in combination with trastuzumab deruxtecan (T-DXd), or in combination with trastuzumab emtansine (T-DM1) in patients with advanced HER2+ metastatic breast cancer (mBC) or metastatic gastric, gastroesophageal junction, or esophageal adenocarcinoma (mGEAC). Patients with brain metastases are eligible for this trial.

### 13.1 Objectives

The primary objectives of dose escalation are:
To characterize the safety, tolerability and the dose-toxicity curve of zongertinib in combination with T-DXd, in combination with trastuzumab with or without capecitabine, or in combination with T-DM1 in patients with HER2+ mBC by assessing escalating dose levels with overdose control to achieve the primary objective of determining the MTDs and/or doses for further development. For mGEAC only the combination of zongertinib with T-DXd is investigated;
To evaluate the number of patients with DLTs within the MTD evaluation period per dose level. The MTD evaluation period is defined as the first 21 days of the first treatment cycle. The MTD is determined by the dose escalation committee (DEC) based on the totality of data. It may be chosen as the highest dose with less than 25% risk of the true DLT rate being equal to or above 33% during the MTD evaluation period based on the BLRM with overdose control (EWOC) for the trial;

The primary characterization of the primary objective is based on the initial dose administered to the patient during the MTD evaluation period. The strategy for handling intercurrent events is a combined composite and principal stratum approach where some intercurrent events are considered as outcome and some define the population consisting of patients who are able to adhere to the assigned treatment regimen and trial schedule.

The primary objectives of dose optimization are:
to assess the anti-tumor activity of zongertinib in the following settings to assist in the selection of optimal dose for further clinical development:
   in combination with T-DM1 in patients with HER2+ mBC (Cohort D)
   in combination with T-DXd in patients with HER2+ mBC (Cohort E)
   in combination with T-DXd in patients with HER2+ mGEAC (Cohort F)
   in combination with capecitabine and trastuzumab in patients with HER2+ mBC (Cohort H)
   as a monotherapy in patients with HER2+ mBC (Cohort I, I-ext)
   in combination with trastuzumab in patients with mBC (Cohort J, J-ext);
the proportion of patients with OR by RECIST version 1.1 as assessed by investigator review in the intent-to-treat population;
the summary measure of OR includes all treated patients regardless of breaks from trial treatment but excludes the effects of any subsequent anti-cancer therapy started before progression.

Secondary objectives include:
To characterize the pharmacokinetic properties of zongertinib when given as monotherapy or in combination (all trial parts);
To further evaluate preliminary efficacy, safety, and the risk-benefit profile of zongertinib monotherapy and zongertinib in combination: with trastuzumab with or without capecitabine, with T-DXd, or with T-DM1 (all trial parts);
To evaluate patient reported outcomes (PROs) (dose optimization).

### 13.2 Endpoints

The primary endpoint of dose escalation is the occurrence of DLTs in the MTD evaluation period. The MTD evaluation period is defined as the first 21 days of the first treatment cycle. The primary endpoint of dose optimization is the OR defined as the best overall response of confirmed CR or confirmed PR according to RECIST 1.1 from the date of treatment start until the earliest date of disease progression, death, or last evaluable tumor assessment before start of subsequent anti-cancer therapy, or treatment discontinuation as assessed by investigator review.

The secondary endpoints of dose escalation are:
OR, as described above;
Occurrence of DLTs during the entire treatment period;
Intensive PK sampling. In particular, the following PK parameters of zongertinib when given in combination are evaluated, if feasible:
   Cₘₐₓ: maximum measured concentration (at steady state);
   AUC_{0-4h,ss}: Area under the concentration-time curve over the time interval from 0 to 4 h at steady state
   AUC_{0-tz,ss}: area under the concentration-time curve over the time interval from 0 to the last quantifiable data point at steady state.

The secondary endpoints of dose optimization are:
PFS, defined as the time from treatment start until the earliest date of tumor progression according to RECIST 1.1 based on investigator review or death from any cause, whichever occurs first;
DC defined as best overall response of CR or PR or SD where best overall response is defined according to RECIST 1.1 from first treatment administration until the earliest of disease progression, death, or last evaluable tumor assessment before start of subsequent anti-cancer therapy, or treatment discontinuation, as assessed by investigator review;
Occurrence of treatment-emergent adverse events leading to zongertinib dose reduction during the on-treatment period;
Sparse PK sampling. In particular, the following PK parameters of zongertinib when given as monotherapy or in combination are evaluated, if feasible:
   Cₘₐₓ₍ₛₛ₎: maximum measured concentration (at steady state);
   AUC_{0-tz,ss}: area under the concentration-time curve over the time interval from 0 to the last quantifiable data point at steady state.
PROs: PRO according to CTCAE, e.g. Mouth/throat sores, Taste changes, Decreased appetite, Nausea, Vomiting, Constipation, Diarrhoea, Shortness of breath, Cough, Rash, Skin dryness, Hair loss, Itching, Numbness & Tingling, Fatigue, Nosebleed, Headache; EORTC IL46 (1 item, overall side effect implant); EORTC IL19 (5 items, physical functioning scale of EORTC QLQ-C30). The time frame is from first administration until an individual patient's end of treatment.

Further endpoints of both parts include:
DOR, defined as the time from first documented confirmed OR until the earliest date of disease progression or death among patients with confirmed objective response, assessed by investigator review;
Tumor shrinkage (in millimeters), defined as the difference between the minimum post-baseline sum of longest diameters of target lesions (longest for non-nodal lesions, short axis for nodal lesions) and the baseline sum of longest diameters of the same set of target lesions;
Further PK parameters of zongertinib and T-DXd/DXd or T-DM1/DM1 when given in combination as feasible and appropriate;
Occurrence of treatment-emergent hospitalizations;
Duration of treatment-emergent hospitalizations.

In addition further endpoints of dose optimization are:
OS, defined as the time from the first treatment administration until death from any cause;
CNS OR according to RANO-BM assessed by investigator review for patients with CNS lesions at baseline. CNS OR is defined as best overall response of CR or PR, where best overall response is determined according to RANO-BM from date of first treatment administration until the earliest of CNS progression, death, or last evaluable tumor assessment before start of subsequent anti-cancer therapy, or treatment discontinuation, as assessed by investigator review;
CNS DC according to RANO-BM assessed by investigator review for patients with CNS lesions at baseline. CNS DC is defined as best overall response of CR or PR or SD, where best overall response is determined according to RANO-BM from date of first treatment administration until the earliest of CNS progression, death, or last evaluable tumor assessment before start of subsequent anti-cancer therapy, or treatment discontinuation, as assessed by investigator review;
Occurrence of treatment-emergent AEs leading to zongertinib permanent discontinuation during the on-treatment period;
Duration of treatment with zongertinib defined as the time from first administration of zongertinib until the last administration of zongertinib.

### 13.3 Design and Trial Population

Approximately 594 patients with histologically or cytologically confirmed unresectable, locally advanced or metastatic HER2+ overexpressing or amplified BC or GEAC who were previously treated with the standard of care (depending on the cohort) can be included in the study. Approximately 92 patients participate in dose escalation, approximately 360 in dose optimization and approximately 70 patients in the extension of certain cohorts and up to 72 patients on dose escalation in backfill cohorts. As soon as the DEC has declared a dose level as safe, the sponsor may decide to open the dose level for additional patients. This is known as ‘backfilling’ of the cohort. Additional patients (up to 7 patients) can be added up to a maximum of 10 patients in total per dose level, to generate safety, tolerability, and efficacy data in a higher number of patients. Patients are divided into cohorts as shown in Table 41.

TABLE 41
Schematic of the clinical trial

| | | |
|---|---|---|
| Cohort | Indication | Therapy |

Dose Escalation (Phase Ib)

| | | |
|---|---|---|
| A | mBC | Zongertinib + T-DM1 |
| B | mBC | Zongertinib + T-DXd |
| C | mGEAC | Zongertinib + T-DXd |
| G | mBC | Zongertinib + trastuzumab + capecitabine |
| K | mBC | Zongertinib + trastuzumab |

Dose Optimization (Phase II)

| | | |
|---|---|---|
| D | mBC | Zongertinib + T-DM1 |
| E | mBC | Zongertinib + T-DXd |
| F | mGEAC | Zongertinib + T-DXd |
| H | mBC | Zongertinib + trastuzumab + capecitabine |
| I | mBC | Zongertinib |
| J | mBC | Zongertinib + trastuzumab |

Phase II extension

| | | |
|---|---|---|
| I-ext | mBC | Zongertinib |
| J-ext | mBC | Zongertinib + trastuzumab |

In Phase Ib, escalating dose levels (DLs) of zongertinib starting at 60 mg and up to a maximum of 360 mg are investigated when co-administered with a fixed dose of T-DM1 for mBC (Cohort A), or a fixed dose of T-DXd for mBC (Cohort B) and mGEAC (Cohort C) or a fixed dose of trastuzumab with capecitabine (Cohort G). Escalating dose levels of zongertinib starting at 180 mg and up to a maximum of 360 mg are investigated when co-administered with a fixed dose of trastuzumab for mBC (Cohort K). Authorised doses of T-DXd, T-DM1, trastuzumab and capecitabine are used. Use of the reduced dose may be decided based on safety data. All cohorts are started in parallel but evaluated separately. While 4 doses are planned for Cohorts A-B-C-G and 2 or 3 dose levels for Cohort K, it is possible that more or fewer doses are investigated according to DEC decisions. Dose escalation is guided by a BLRM with EWOC for each cohort individually.

In Phase II, two dose levels to be tested per indication and treatment regimen (Cohorts D-E-F-H-J) are chosen based on the data from the dose escalation part (Cohorts A-B-C-G-K). The lower dose is referred to as DL1; the higher dose is referred to as DL2. For Cohort I, the dose at DL1 is 180 mg and DL2 is 360 mg.

### 13.4 Inclusion and Exclusion Criteria

Patients with histologically or cytologically confirmed unresectable, locally advanced or metastatic HER2+ overexpressing or amplified BC or GEAC who were previously treated with the standard of care (depending on the cohort) can be screened for the trial.

Main inclusion criteria are:
Patients ≥18 years of age or over the legal age of consent in countries where that is greater than 18 years at the time of signature of the informed consent form (ICF);
Documented HER2+ mBC or mGEAC according to ASCO-CAP guidelines for the respective cancer indication according to the result of local testing from the latest available biopsy (e.g. defined as immunohistochemistry [IHC] 3+ or IHC 2+ and evidence of HER2 amplification by in situ hybridization [ISH]);
For dose optimization (Phase II): Patient must provide tumor tissue from locations not radiated prior to biopsy, if possible, collected through archival tissue;
Documented investigator assessed progression after HER2 directed treatment for unresectable locally advanced or metastatic disease. For cohorts Cohorts D, H, I, (I-ext), J (and J-ext)—patients must have been pretreated with T-DXd and have progressed or have been intolerant to previous T-DXd);
Presence of at least one measurable lesion according to RECIST 1.1;
Eastern Cooperative Oncology Group (ECOG) score of 0 or 1;
Adequate organ function based on laboratory values.

Main exclusion criteria are:
Previous treatment with:
   Any small molecule HER2 inhibitor in the advanced or metastatic setting in Cohorts D, E, F, and H. In Cohort D allowed in up to 15 patients in each DL;
   T-DXd in Cohorts E and F;
   T-DM1 in Cohort D and H. In Cohort H allowed in up to 15 patients in each DL;
   Capecitabine in Cohort D and H. In Cohort D allowed in up to 15 patients in each Dose level (DL);
Presence of uncontrolled and/or symptomatic brain metastases, or leptomeningeal disease. Patients with known CNS lesions must not have any of the following:
   Any untreated brain lesions >2.0 cm in size, unless discussed with medical monitor and approval for enrolment is given
   Ongoing use of systemic corticosteroids for control of symptoms of brain metastases at a total daily dose of >2 mg of dexamethasone (or equivalent)
   However, patients on a chronic stable dose of ≤2 mg total daily of dexamethasone (or equivalent) may be eligible with discussion and approval by the medical monitor
   Any brain lesion thought to require immediate local therapy, including (but not limited to) a lesion in an anatomic site where increase in size or possible treatment-related edema may pose risk to patient (e.g. brain stem lesions).
   Patients who undergo local treatment for such lesions identified by screening contrast brain MRI may still be eligible for the study if treated with CNS local therapy for newly identified lesions found on contrast brain MRI performed during screening for this study if all of the following criteria are met:
   Time since WBRT is ≥21 days prior to first dose of treatment, time since SRS is ≥7 days prior to first dose of treatment, or time since surgical resection is ≥28 days
   Other sites of disease assessable by RECIST 1.1 are present
Known or suspected leptomeningeal disease as documented by the investigator
Have poorly controlled (>1/week) generalized or complex partial seizures, or manifest neurologic progression due to brain metastases notwithstanding CNS-directed therapy;
Radiotherapy within 4 weeks prior to randomization except for palliative radiotherapy for symptomatic metastasis within 2 weeks prior to randomization, but this must be discussed with the sponsor
Mean resting corrected QT interval (QTcF)>470 msec.;
Any factors that increase the risk of QTc prolongation or risk of arrhythmic events such as heart failure, hypokalaemia, congenital long QT syndrome, personal or family history of long QT syndrome or unexplained sudden death under 40 years-of-age, or any concomitant medication known to prolong the QT interval;
Ejection fraction <50% or the lower limit of normal of the institutional standard within 28 days prior to randomization;
History of (non-infectious) interstitial lung disease (ILD)/pneumonitis that required steroids, current ILD/pneumonitis, or where suspected ILD/pneumonitis cannot be ruled out by imaging at screening.

Additional inclusion and exclusion criteria may apply.

### 13.5 Treatments

Zongertinib is administered orally from 60 mg QD up to 360 mg QD. T-DM1 is administered as an intravenous infusion of 3.6 mg/kg to mBC patients once every 3 weeks (21-day cycle). T-DXd is administered as an intravenous infusion of 5.4 mg/kg to mBC patients or 6.4 mg/kg to mGAEC patients once every 3 weeks (21-day cycle). Capecitabine 1000 mg/m2 is administered orally BID on days 1-14 of each 21-day cycle. If patients have not received trastuzumab within 4 weeks of the first day of the cycle, a loading dose of 8 mg/kg intravenously is administered, followed by once every 3 weeks (21-day cycle) 6 mg/kg.

If patients have received trastuzumab within 4 weeks of the first day of the cycle, 6 mg/kg intravenously once every 3 weeks (21-day cycle) is administered.

T-DM1, T-DXd, trastuzumab and capecitabine are administered according to the approved product label.

All patients are treated in cycles of 3 weeks (21 days). Patients may continue treatment until documented disease progression according to RECIST 1.1, undue toxicity, or other criteria for stopping treatment as defined in the full clinical trial protocol (CTP) are met.

### 13.6 Assessments

Tumor assessments should include computed tomography (CT) scans of the chest, abdomen, pelvis (or PET/CT) and a brain MRI at screening. If clinically indicated, imaging of any other known or suspected sites of disease (e.g. bone) using an appropriate method (CT scan, MRI, PET/CT, or bone scan) should be performed. The same radiographic procedure must be used throughout the trial. If a patient does not have brain metastases at the screening visit there is no need to perform brain MRI during following visits unless brain metastases are suspected. Assessments are performed by the investigator at:
Screening (≤28 days prior to initiation of treatment (Note: assessments performed prior to informed consent as part of routine clinical practice are accepted if they meet the requirements of the protocol and are performed within the screening visit window);
Every 6 weeks ±5 days calculated from day 1 of cycle 1 until PD/start of subsequent anti-cancer treatment; after 18 months, the frequency will be reduced to every 12 weeks (±5 days);
EoT visit (if not performed within the previous 3 weeks). Patients who discontinue trial treatment between two clinical visits should undergo the End of Treatment (EoT) visit as soon as possible. If the decision is made to permanently discontinue study treatment during a scheduled visit, the end of treatment (EoT) visit should be performed instead of the scheduled visit;
For patients without objective progression at the EoT visit, every 6 weeks (+5 days) thereafter until progression, or until one of the following: death, lost to follow up, start of subsequent anti-cancer therapy, patient's end of study, withdrawal of consent or until the end of the trial.
Additional scans may also be taken outside of scheduled visits if progression is suspected and at the discretion of the investigator.

Tumor response by RECIST 1.1 and RANO-BM can be evaluated as described in Example 1. Safety assessments were performed in all treated patients using physical examinations, assessments of vital signs, safety laboratory parameters, electrocardiograms, left ventricular ejection function, and by monitoring the occurrence of adverse events. Adverse events were assessed according to the National Cancer Institute CTCAE version 5.0.

### 13.7 Statistical Methods

Tumor assessments are evaluated similarly to Example 1. The analyses are descriptive and exploratory. No formal statistical test is performed. All analyses are performed separately for each cohort. For each cohort in the Phase Ib part, dose escalation is guided by a Bayesian logistic regression model (BLRM) with overdose control that is fitted to binary toxicity outcomes. The estimates of parameters are updated as data are accumulated using the BLRM. At the end of dose escalation, the toxicity probability at each dose level is calculated to determine an estimate of the MTD for each cohort.

1. A method for the treatment of metastatic cancer, comprising administering to a patient in need thereof a therapeutically effective amount of zongertinib or a pharmaceutically acceptable salt thereof, wherein the metastatic cancer comprises metastatic cancer located in the central nervous system.

2. The method of claim 1, wherein the metastatic cancer comprises metastatic cancer located in the brain.

3. The method of claim 1, wherein the metastatic cancer is selected from the group consisting of: metastatic brain cancer, metastatic breast cancer, metastatic biliary tract cancer, metastatic bladder cancer, metastatic cervical cancer, metastatic uterine cancer, metastatic colorectal cancer, metastatic endometrial cancer, metastatic ovarian cancer, metastatic skin cancer, metastatic gastric cancer, metastatic esophagus tumor, metastatic head and neck tumor, metastatic salivary gland cancer, metastatic gastrointestinal cancer, metastatic small bowel cancer, metastatic gallbladder tumor, metastatic kidney cancer, metastatic liver cancer, metastatic lung cancer and metastatic prostate cancer.

4. The method of claim 1, wherein the metastatic cancer is metastatic lung cancer or metastatic breast cancer.

5. The method of claim 4, wherein the metastatic cancer is metastatic non-small cell lung cancer (NSCLC).

6. The method of claim 1, wherein the metastatic cancer comprises a HER2 aberration.

7. The method of claim 1, wherein the metastatic cancer is HER2 mutant.

8. The method of claim 1, wherein the metastatic cancer comprises a mutation in the tyrosine kinase domain of HER2.

9. The method of claim 1, wherein the metastatic cancer is asymptomatic, symptomatic, stable or active.

10. The method of claim 1, wherein zongertinib or the pharmaceutically acceptable salt thereof is administered as second or further line therapy.

11. The method of claim 1, wherein zongertinib or the pharmaceutically acceptable salt thereof is administered following a systemic anti-cancer therapy.

12. The method of claim 1, wherein zongertinib or the pharmaceutically acceptable salt thereof is administered in a daily dose of 120 mg, 180 mg, 200 mg, 240 mg, 300 mg, 360 mg, 400 mg, 420 mg, 480 mg, 500 mg, 540 mg or 600 mg.

13. The method of claim 1, wherein zongertinib or the pharmaceutically acceptable salt thereof is administered in a daily dose of 120 mg or 240 mg.

14. The method of claim 1, wherein zongertinib or the pharmaceutically acceptable salt thereof is administered as monotherapy.

15. The method of claim 1, wherein zongertinib or the pharmaceutically acceptable salt thereof is administered in combination with a combination partner.

16. The method of claim 1, wherein zongertinib or the pharmaceutically acceptable salt thereof is administered as a spray-dried dispersion.
